(19) United States
(12) Patent Application Publication
(10) Pub. No.: US 2024/0317735 A1
(43) Pub. Date: Sep. 26, 2024

(54) HYDROISOQUINOLINE OR HYDRONAPHTHYRIDINE COMPOUNDS FOR THE TREATMENT OF AUTOIMMUNE DISEASE

(71) Applicant: Hoffmann-La Roche Inc., 8 - Legal Department Little Falls,, NJ (US)

(72) Inventors: Fabian DEY, Zuerich (CH); Xiaoqing WANG, Shanghai (CN); Zhisen ZHANG, Shanghai (CN); Wei ZHU, Shanghai (CN)

(73) Assignee: Hoffmann-La Roche Inc., 8 - Legal Department Little Falls, NJ (US)

(21) Appl. No.: 18/016,289

(22) PCT Filed: Jul. 12, 2021
(86) PCT No.: PCT/EP2021/069294

§ 371 Date: Jan. 13, 2023

(30) Foreign Application Priority Data
Jul. 14, 2020 (WO) PCT/CN2020/101904
May 17, 2021 (WO) PCT/CN2021/094138

(51) Int. Cl. C07D 471/04; A61K 31/4375; A61K 31/4545; A61K 31/4725; A61K 31/496; A61K 31/4985; A61K 31/4995; A61K 31/5377; A61K 31/5386; C07D 519/00

(52) U.S. Cl. CPC C07D 471/04; A61K 31/4375; A61K 31/4545; A61K 31/4725; A61K 31/496; A61K 31/4985; A61K 31/4995; A61K 31/5377; A61K 31/5386; C07D 519/00

(57) ABSTRACT

The present invention relates to compounds of formula (I),

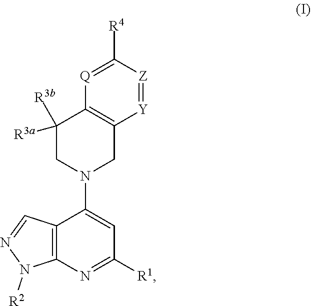

wherein R¹, R², R^{3a}, R^{3b}, R⁴, Q, Z and Y are as described herein, and their pharmaceutically acceptable salt thereof, and compositions including the compounds and methods of using the compounds.

### CROSS-REFERENCE TO RELATED APPLICATIONS

This application is the National Stage Entry under 35 U.S.C. § 371 of International Application No. PCT/EP2021/069294, filed Jul. 12, 2021, which claims benefit of priority under 35 U.S.C. § 119(a) to China Application No. PCT/CN2020/101904 filed on Jul. 14, 2020, and to China Application No. PCT/CN2021/094138 filed May 17, 2021, each of which is incorporated herein by reference in its entirety.

### FIELD OF THE INVENTION

The present invention relates to organic compounds useful for therapy and/or prophylaxis in a mammal, and in particular to antagonist of TLR7 and/or TLR8 and/or TLR9 useful for treating systemic lupus erythematosus or lupus nephritis.

### BACKGROUND OF THE INVENTION

Autoimmune connective tissue disease (CTD) include prototypical autoimmune syndromes such as Systemic Lupus Erythematosus (SLE), primary Sjögren's syndrome (pSjS), mixed connective tissue disease (MCTD), Dermatomyositis/Polymyositis (DM/PM), Rheumatoid Arthritis (RA), and systemic sclerosis (SSc). With the exception of RA, no really effective and safe therapies are available to patients. SLE represents the prototypical CTD with a prevalence of 20-150 per 100,000 and causes broad inflammation and tissue damage in distinct organs, from commonly observed symptoms in the skin and joints to renal, lung, or heart failure. Traditionally, SLE has been treated with nonspecific anti-inflammatory or immunosuppressive drugs. However, long-term usage of immunosuppressive drug, e.g. corticosteroids is only partially effective, and is associated with undesirable toxicity and side effects. Belimumab is the only FDA-approved drug for lupus in the last 50 years, despite its modest and delayed efficacy in only a fraction of SLE patients (Navarra, S. V. et al Lancet 2011, 377, 721). Other biologics, such as anti-CD20 mAbs, mAbs against or soluble receptors of specific cytokines, have failed in most clinical studies. Thus, novel therapies are required that provide sustained improvement in a greater proportion of patient groups and are safer for chronic use in many autoimmune as well as auto-inflammation diseases.

Toll like Receptors (TLR) are an important family of pattern recognition receptors (PRR) which can initiate broad immune responses in a wide variety of immune cells. As natural host defense sensors, endosomal TLRs 7, 8 and 9 recognize nucleic acids derived from viruses, bacteria; specifically, TLR7/8 and TLR9 recognize single-stranded RNA (ssRNA) and single-stranded CpG-DNA, respectively. However, aberrant nucleic acid sensing of TRL7, 8, 9 is considered as a key node in a broad of autoimmune and auto-inflammatory diseases (Krieg, A. M. et al. Immunol. Rev. 2007, 220, 251. Jiménez-Dalmaroni, M. J. et al Autoimmun Rev. 2016, 15, 1. Chen, J. Q., et al. Clinical Reviews in Allergy & Immunology 2016, 50, 1). Anti-RNA and anti-DNA antibodies are well-established diagnostic markers of SLE, and these antibodies can deliver both self-RNA and self-DNA to endosomes. While self-RNA complexes can be recognized by TLR7 and TLR8, self-DNA complexes can trigger TLR9 activation. Indeed, defective clearance of self-RNA and self-DNA from blood and/or tissues is evident in SLE (Systemic Lupus Erythematosus) patients. TLR7 and TLR9 have been reported to be upregulated in SLE tissues, and correlate with chronicity and activity of lupus nephritis, respectively. In B cells of SLE patients, TLR7 expression correlates with anti-RNP antibody production, while TLR9 expression with IL-6 and anti-dsDNA antibody levels. Consistently, in lupus mouse models, TLR7 is required for anti-RNA antibodies, and TLR9 is required for anti-nucleosome antibody. On the other hand, overexpression of TLR7 or human TLR8 in mice promotes autoimmunity and autoinflammation. Moreover, activation of TLR8 specifically contributes to inflammatory cytokine secretion of mDC/macrophages, neutrophil NETosis, induction of Th17 cells, and suppression of Treg cells. In addition to the described role of TLR9 in promoting autoantibody production of B cells, activation of TLR9 by self-DNA in pDC also leads to induction of type I IFNs and other inflammatory cytokines. Given these roles of TLR9 in both pDC and B cells, both as key contributors to the pathogenesis of autoimmune diseases, and the extensive presence of self-DNA complexes that could readily activate TLR9 in many patients with autoimmune diseases, it may have extra benefit to further block self-DNA mediated TLR9 pathways on top of inhibition of TLR7 and TLR8 pathways. Taken together, TLR7, 8 and 9 pathways represent new therapeutic targets for the treatment of autoimmune and auto-inflammatory diseases, for which no effective steroid-free and non-cytotoxic oral drugs exist, and inhibition of all these pathways from the very upstream may deliver satisfying therapeutic effects. As such, we invented oral compounds that target and suppress TLR7, TLR8 and TLR9 for the treatment of autoimmune and auto-inflammatory diseases.

### SUMMARY OF THE INVENTION

The present invention relates to novel compounds of formula (I),

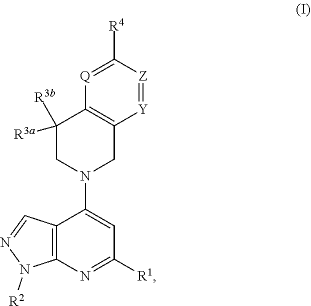

wherein
R¹ is H or C₁₋₆alkyl;
R² is C₁₋₆alkyl;
R^{3a} is H or C₁₋₆alkyl;
R^{3b} is H or C₁₋₆alkyl;
R⁴ is (C₁₋₆alkylpiperazinyl)C₁₋₆alkoxy,
   (C₁₋₆alkyl)₂piperazin-4-iumyl,
   (formylpiperazinyl)C₁₋₆alkoxy,
   1,2,3,4,6,7,8,8a-octahydropyrrolo[1,2-a]pyrazinyloxy,
   2-oxo-1-oxa-3,7-diazaspiro[4.4]nonanyl,
   2-oxo-1-oxa-3,8-diazaspiro[4.5]decanyl,
   3,8-diazabicyclo[3.2.1]octanyl,
   3,9-diazaspiro[5.5]undecanyl,
   3-oxo-1,5,6,7,8,8a-hexahydroimidazo[1,5-a]pyrazinyl,
   3-oxo-2,7-diazaspiro[4.4]nonanyl,
   3-oxo-2,8-diazaspiro[4.5]decanyl,
   3-oxo-3a,4,5,6,7,7a-hexahydro-1H-pyrrolo[3,4-c]pyridinyl,
   4,7-diazaspiro[2.5]octanyl,
   4-oxo-1,2,3,3a,6,6a-hexahydropyrrolo[3,4-c]pyrrolyl,
   4-piperidinylazetidinyloxy,
   5-oxa-2,8-diazaspiro[3.5]nonanyl,
   7-oxo-2,6-diazaspiro[3.4]octanyl,
   piperazinyl,
   piperazinylC₁₋₆alkoxy,
   piperidinyl,
   piperidinyloxy, or
   pyrrolidinyl,
   wherein
      piperazinyl is unsubstituted or substituted by (C₁₋₆alkyl)₂aminoC₁₋₆alkyl, 1,2,3,4-tetrahydro-2,6-naphthyridinyl, C₁₋₆alkoxyC₁₋₆alkyl, C₁₋₆alkyl, C₁₋₆alkylimidazolylC₁₋₆alkyl, hydroxyC₁₋₆alkyl, morpholinylcarbonyl, piperidinylcarbonyl, pyrrolidinylcarbonyl or pyrrolidinylsulfonyl,
      piperidinyl is once or twice substituted by substituents independently selected from halogen, amino, amino(hydroxy)piperidinyl, amino(C₁₋₆alkoxy)pyrrolidinyl, aminoazetidinyl, C₁₋₆alkyl, C₁₋₆alkoxy, C₁₋₆alkyl)₂amino, hydroxy, ((C₁₋₆alkyl)₂amino)C₁₋₆alkyl and piperazinyl, pyrrolidinyl is twice or three times substituted by substituents independently
      selected from halogen, amino, hydroxy, C₁₋₆alkyl and C₁₋₆alkoxy;
Q and Z are independently selected from CH and N;
Y is CH;
or a pharmaceutically acceptable salt thereof.

Another object of the present invention is related to novel compounds of formula (I) or (Ia). Their manufacture, medicaments based on a compound in accordance with the invention and their production as well as the use of compounds of formula (I) or (Ia) as TLR7 and TLR8 and TLR9 antagonist, and for the treatment or prophylaxis of systemic lupus erythematosus or lupus nephritis. The compounds of formula (I) or (Ia) show superior TLR7 and TLR8 and TLR9 antagonism activity. In addition, the compounds of formula (I) or (Ia) also show good cytotoxicity, phototoxicity, solubility, hPBMC, human microsome stability and SDPK profiles, as well as low CYP inhibition.

Novartis patent WO2018047081 disclosed compounds with same pyrazolo[3,4-b]pyridinyl moiety as the compounds of this invention, however the central bicyclic core of 4,5,6,7-tetrahydro-1H-pyrazolo[4,3-c]pyridine and the terminal substitution with bicyclo[2,2,2]octane/bicyclo[1,1,1]pentane moieties were essential for the TLR7/8/9 activity based on the information disclosed in Novartis patent, which also were considered as the major structural differentiation compared with the compounds of current invention. On the other hand, most of the compounds in WO2018047081 showed poor TLR9 activity. Unfortunately few compounds with relatively improved TLR9 activity, such as N79 (as Example 79) with best TLR9 activity, still suffer from poor human liver microsome stability (see Table 5) and therefore have unsatisfactory PK profile.

Another Novartis patent WO2019220390 disclosed the polymorph of compound N8 (as Example 8 in WO2018047081), which considered as the lead compound of its series and turned out to have much lower TLR9 activity and similar poor human liver microsome stability (see Table 5).

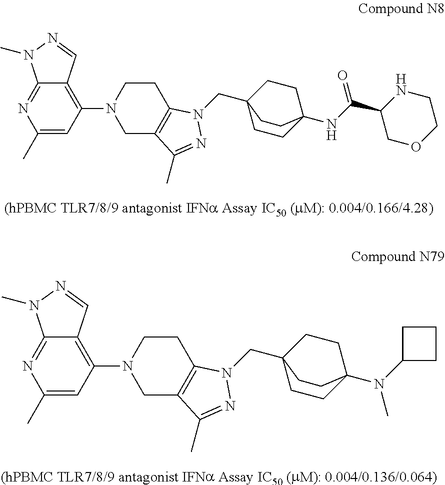 [SMILES: Cc1cc(N2CCc3c(c(C)nn3CC34CCC(NC(=O)[C@@H]5COCCN5)(CC3)CC4)C2)c2cnn(C)c2n1 ; Cc1cc(N2CCc3c(c(C)nn3CC34CCC(N(C)C5CCC5)(CC3)CC4)C2)c2cnn(C)c2n1]

### DETAILED DESCRIPTION OF THE INVENTION

### Definitions

The term “C₁₋₆alkyl” denotes a saturated, linear or branched chain alkyl group containing 1 to 6, particularly 1 to 4 carbon atoms, for example methyl, ethyl, n-propyl, isopropyl, n-butyl, isobutyl, tert-butyl and the like. Particular “C₁₋₆alkyl” groups are methyl, ethyl and n-propyl.

The term “C₁₋₆alkoxy” denotes C₁₋₆alkyl-O—.

The term “oxy” denotes —O—. Example such as 1,2,3,4,6,7,8,8a-octahydropyrrolo[1,2-a]pyrazinyloxy refers to 1,2,3,4,6,7,8,8a-octahydropyrrolo[1,2-a]pyrazinyl-O—.

The term “oxo” denotes═O.

The term “halogen” and “halo” are used interchangeably herein and denote fluoro, chloro, bromo, or iodo.

The term “aryl” denotes an aromatic hydrocarbon mono- or bicyclic ring system of 5 to 12 ring atoms. Examples of aryl include, but not limited to, phenyl and naphthyl. Aryl can be further substituted by substituents includes, but not limited to C₁₋₆alkyl; 3,4,4a,5,7,7a-hexahydro-2H-pyrrolo[3,4-b][1,4]oxazinyl; 1,4-diazepanyl; 2,6-diazaspiro[3.3]heptanyl substituted by C₁₋₆alkyl; 5-oxa-2,8-diazaspiro[3.5]nonanyl; amino-1,4-oxazepanyl; azetidinyl substituted by one or two substituents independently selected from amino and C₁₋₆alkyl; piperazinyl unsubstituted or substituted by C₁₋₆alkyl; and pyrrolidinyl substituted by one or two substituents independently selected from amino, C₁₋₆alkoxy and halogen.

The term “cis” and “trans” denote the relative stereochemistry of the molecule or moiety. For example: starting material of Example 17, cis-4-oxo-hexahydro-pyrrolo[3,4-c]pyrrole-2-carboxylic acid tert-butyl ester

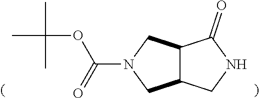

refers to a mixture of

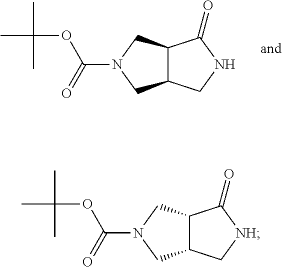 [SMILES: CC(C)(C)OC(=O)N1C[C@@H]2CNC(=O)[C@@H]2C1 ; CC(C)(C)OC(=O)N1C[C@H]2CNC(=O)[C@H]2C1]

similarly, starting material of Example 29, trans-tert-butyl N-[3-hydroxy-4-piperidyl]carbamate

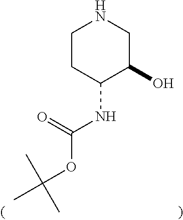

refers to a mixture of and

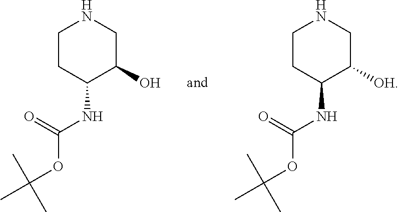

The way of showing relative stereochemistry also applies to the final compounds.

The term “pharmaceutically acceptable salts” denotes salts which are not biologically or otherwise undesirable. Pharmaceutically acceptable salts include both acid and base addition salts.

The term “pharmaceutically acceptable acid addition salt” denotes those pharmaceutically acceptable salts formed with inorganic acids such as hydrochloric acid, hydrobromic acid, sulfuric acid, nitric acid, carbonic acid, phosphoric acid, and organic acids selected from aliphatic, cycloaliphatic, aromatic, araliphatic, heterocyclic, carboxylic, and sulfonic classes of organic acids such as formic acid, acetic acid, propionic acid, glycolic acid, gluconic acid, lactic acid, pyruvic acid, oxalic acid, malic acid, maleic acid, maloneic acid, succinic acid, fumaric acid, tartaric acid, citric acid, aspartic acid, ascorbic acid, glutamic acid, anthranilic acid, benzoic acid, cinnamic acid, mandelic acid, embonic acid, phenylacetic acid, methanesulfonic acid, ethanesulfonic acid, p-toluenesulfonic acid, and salicyclic acid.

The term “pharmaceutically acceptable base addition salt” denotes those pharmaceutically acceptable salts formed with an organic or inorganic base. Examples of acceptable inorganic bases include sodium, potassium, ammonium, calcium, magnesium, iron, zinc, copper, manganese, and aluminum salts. Salts derived from pharmaceutically acceptable organic nontoxic bases includes salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-diethylaminoethanol, trimethamine, dicyclohexylamine, lysine, arginine, histidine, caffeine, procaine, hydrabamine, choline, betaine, ethylenediamine, glucosamine, methylglucamine, theobromine, purines, piperizine, piperidine, N-ethylpiperidine, and polyamine resins.

The term “A pharmaceutically active metabolite” denotes a pharmacologically active product produced through metabolism in the body of a specified compound or salt thereof. After entry into the body, most drugs are substrates for chemical reactions that may change their physical properties and biologic effects. These metabolic conversions, which usually affect the polarity of the compounds of the invention, alter the way in which drugs are distributed in and excreted from the body. However, in some cases, metabolism of a drug is required for therapeutic effect.

The term “therapeutically effective amount” denotes an amount of a compound or molecule of the present invention that, when administered to a subject, (i) treats or prevents the particular disease, condition or disorder, (ii) attenuates, ameliorates or eliminates one or more symptoms of the particular disease, condition, or disorder, or (iii) prevents or delays the onset of one or more symptoms of the particular disease, condition or disorder described herein. The therapeutically effective amount will vary depending on the compound, the disease state being treated, the severity of the disease treated, the age and relative health of the subject, the route and form of administration, the judgement of the attending medical or veterinary practitioner, and other factors.

The term “pharmaceutical composition” denotes a mixture or solution comprising a therapeutically effective amount of an active pharmaceutical ingredient together with pharmaceutically acceptable excipients to be administered to a mammal, e.g., a human in need thereof.

### Antagonist of TLR7 and TLR8 and TLR9

The present invention relates to (i) a compound of formula (I),

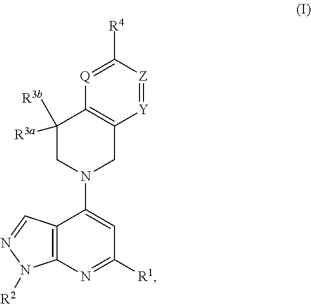

wherein
R¹ is H or C₁₋₆alkyl;
R² is C₁₋₆alkyl;
R^{3a} is H or C₁₋₆alkyl;
R^{3b} is H or C₁₋₆alkyl;
R⁴ is (C₁₋₆alkylpiperazinyl)C₁₋₆alkoxy,
   (C₁₋₆alkyl)₂piperazin-4-iumyl,
   (formylpiperazinyl)C₁₋₆alkoxy,
   1,2,3,4,6,7,8,8a-octahydropyrrolo[1,2-a]pyrazinyloxy,
   2-oxo-1-oxa-3,7-diazaspiro[4.4]nonanyl,
   2-oxo-1-oxa-3,8-diazaspiro[4.5]decanyl,
   3,8-diazabicyclo[3.2.1]octanyl,
   3,9-diazaspiro[5.5]undecanyl,
   3-oxo-1,5,6,7,8,8a-hexahydroimidazo[1,5-a]pyrazinyl,
   3-oxo-2,7-diazaspiro[4.4]nonanyl,
   3-oxo-2,8-diazaspiro[4.5]decanyl,
   3-oxo-3a,4,5,6,7,7a-hexahydro-1H-pyrrolo[3,4-c]pyridinyl,
   4,7-diazaspiro[2.5]octanyl,
   4-oxo-1,2,3,3a,6,6a-hexahydropyrrolo[3,4-c]pyrrolyl,
   4-piperidinylazetidinyloxy,
   5-oxa-2,8-diazaspiro[3.5]nonanyl,
   7-oxo-2,6-diazaspiro[3.4]octanyl,
   piperazinyl,
   piperazinylC₁₋₆alkoxy,
   piperidinyl,
   piperidinyloxy, or
   pyrrolidinyl,
   wherein
      piperazinyl is unsubstituted or substituted by (C₁₋₆alkyl)₂aminoC₁₋₆alkyl, 1,2,3,4-tetrahydro-2,6-naphthyridinyl, C₁₋₆alkoxyC₁₋₆alkyl, C₁₋₆alkyl, C₁₋₆alkylimidazolylC₁₋₆alkyl, hydroxyC₁₋₆alkyl, morpholinylcarbonyl, piperidinylcarbonyl, pyrrolidinylcarbonyl or pyrrolidinylsulfonyl,
      piperidinyl is once or twice substituted by substituents independently selected from halogen, amino, amino(hydroxy)piperidinyl, amino(C₁₋₆alkoxy)pyrrolidinyl, aminoazetidinyl, C₁₋₆alkyl, C₁₋₆alkoxy, C₁₋₆alkyl)₂amino, hydroxy, ((C₁₋₆alkyl)₂amino)C₁₋₆alkyl and piperazinyl,
      pyrrolidinyl is twice or three times substituted by substituents independently selected from halogen, amino, hydroxy, C₁₋₆alkyl and C₁₋₆alkoxy;
Q and Z are independently selected from CH and N;
Y is CH;
or a pharmaceutically acceptable salt thereof.

Another embodiment of present invention is (ii) a compound of formula (Ia),

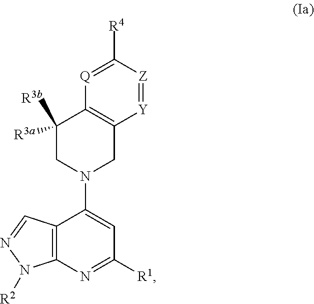

wherein
R¹ is H or C₁₋₆alkyl;
R² is C₁₋₆alkyl;
R^{3a} is H or C₁₋₆alkyl;
R^{3b} is H or C₁₋₆alkyl;
R⁴ is (C₁₋₆alkylpiperazinyl)C₁₋₆alkoxy,
   (C₁₋₆alkyl)₂piperazin-4-iumyl,
   (formylpiperazinyl)C₁₋₆alkoxy,
   1,2,3,4,6,7,8,8a-octahydropyrrolo[1,2-a]pyrazinyloxy,
   2-oxo-1-oxa-3,7-diazaspiro[4.4]nonanyl,
   2-oxo-1-oxa-3,8-diazaspiro[4.5]decanyl,
   3,8-diazabicyclo[3.2.1]octanyl,
   3,9-diazaspiro[5.5]undecanyl,
   3-oxo-1,5,6,7,8,8a-hexahydroimidazo[1,5-a]pyrazinyl,
   3-oxo-2,7-diazaspiro[4.4]nonanyl,
   3-oxo-2,8-diazaspiro[4.5]decanyl,
   3-oxo-3a,4,5,6,7,7a-hexahydro-1H-pyrrolo[3,4-c]pyridinyl,
   4,7-diazaspiro[2.5]octanyl,
   4-oxo-1,2,3,3a,6,6a-hexahydropyrrolo[3,4-c]pyrrolyl,
   4-piperidinylazetidinyloxy,
   5-oxa-2,8-diazaspiro[3.5]nonanyl,
   7-oxo-2,6-diazaspiro[3.4]octanyl,
   piperazinyl,
   piperazinylC₁₋₆alkoxy,
   piperidinyl,
   piperidinyloxy, or
   pyrrolidinyl,
   wherein
      piperazinyl is unsubstituted or substituted by (C₁₋₆alkyl)₂aminoC₁₋₆alkyl, 1,2,3,4-tetrahydro-2,6-naphthyridinyl, C₁₋₆alkoxyC₁₋₆alkyl, C₁₋₆alkyl, C₁₋₆alkylimidazolylC₁₋₆alkyl, hydroxyC₁₋₆alkyl, morpholinylcarbonyl, piperidinylcarbonyl, pyrrolidinylcarbonyl or pyrrolidinylsulfonyl,
      piperidinyl is once or twice substituted by substituents independently selected from halogen, amino, amino(hydroxy)piperidinyl, amino(C₁₋₆alkoxy)pyrrolidinyl, aminoazetidinyl, C₁₋₆alkyl, C₁₋₆alkoxy, C₁₋₆alkyl)₂amino, hydroxy, ((C₁₋₆alkyl)₂amino)C₁₋₆alkyl and piperazinyl,
      pyrrolidinyl is twice or three times substituted by substituents independently selected from halogen, amino, hydroxy, C₁₋₆alkyl and C₁₋₆alkoxy;
Q and Z are independently selected from CH and N;
Y is CH;
or a pharmaceutically acceptable salt thereof.

A further embodiment of present invention is (iii) a compound of formula (I) or (Ia) according to (i) or (ii), or a pharmaceutically acceptable salt thereof, wherein Q is CH or N; Z is CH or N; and Y is CH; with the proviso that Q and Z are not N simultaneously.

A further embodiment of present invention is (iv) a compound of formula (I) or (Ia), according to any one of (i) to (iii), or a pharmaceutically acceptable salt thereof, wherein R⁴ is (1-C₁₋₆alkylpiperazin-2-yl)C₁₋₆alkoxy, (C₁₋₆alkyl)₂piperazin-4-ium-1-yl, (1-formylpiperazin-2-yl)C₁₋₆alkoxy, 1,2,3,4,6,7,8,8a-octahydropyrrolo[1,2-a]pyrazin-7-yloxy, 2-oxo-1-oxa-3,7-diazaspiro[4.4]nonan-3-yl, 2-oxo-1-oxa-3,8-diazaspiro[4.5]decan-3-yl, 3,8-diazabicyclo[3.2.1]octan-8-yl, 3,9-diazaspiro[5.5]undecan-3-yl, 3-oxo-1,5,6,7,8,8a-hexahydroimidazo[1,5-a]pyrazin-2-yl, 3-oxo-2,7-diazaspiro[4.4]nonan-2-yl, 3-oxo-2,8-diazaspiro[4.5]decan-2-yl, 3-oxo-3a,4,5,6,7,7a-hexahydro-1H-pyrrolo[3,4-c]pyridin-2-yl, 4-oxo-1,2,3,3a,6,6a-hexahydropyrrolo[3,4-c]pyrrol-5-yl, 4-piperidylazetidin-3-yloxy, 5-oxa-2,8-diazaspiro[3.5]nonan-2-yl, 7-oxo-2,6-diazaspiro[3.4]octan-6-yl, piperazin-1-yl, 1,2,3,4-tetrahydro-2,6-naphthyridin-7-yl-piperazin-1-yl, (1-C₁₋₆alkylimidazol-4-ylC₁₋₆alkyl)piperazin-1-yl, (hydroxyC₁₋₆alkyl)piperazin-1-yl, C₁₋₆alkyl-piperazin-1-yl, (morpholin-2-ylcarbonyl)piperazin-1-yl, (C₁₋₆alkyl)₂aminoC₁₋₆alkyl-piperazin-1-yl, piperidin-4-ylcarbonyl-piperazin-1-yl, pyrrolidin-2-ylcarbonyl-piperazin-1-yl, pyrrolidin-3-ylsulfonyl-piperazin-1-yl, (C₁₋₆alkoxyC₁₋₆alkyl)piperazin-1-yl, piperazin-1-ylC₁₋₆alkoxy, piperazin-2-ylC₁₋₆alkoxy, (3-amino-4-C₁₋₆alkoxy-pyrrolidin-1-yl)-1-piperidinyl, 3-aminoazetidin-1-yl-1-piperidinyl, (4-amino-3-hydroxy-1-piperidinyl)-1-piperidinyl, 4-hydroxy-4-((C₁₋₆alkyl)₂amino)C₁₋₆alkyl-1-piperidinyl, piperazin-1-yl-1-piperidinyl, amino-1-piperidinyl, 4-amino-4-C₁₋₆alkyl-1-piperidinyl, (C₁₋₆alkyl)₂amino-1-piperidinyl, 4-amino-3-methoxy-1-piperidinyl, 4-amino-3-halo-1-piperidinyl, 3-amino-4-C₁₋₆alkoxy-pyrrolidin-1-yl, 4-amino-3-hydroxy-pyrrolidin-1-yl, 3-amino-4-halo-pyrrolidin-1-yl or 4-amino-3-hydroxy-3-C₁₋₆alkyl-pyrrolidin-1-yl.

A further embodiment of present invention is (v) a compound of formula (I) or (Ia) according to any one of (i) to (iv), wherein R⁴ is 3-amino-4-C₁₋₆alkoxy-pyrrolidin-1-yl, 4-amino-3-hydroxy-pyrrolidin-1-yl, 3-aminoazetidin-1-yl-1-piperidinyl, 4-amino-3-C₁₋₆alkoxy-1-piperidinyl, 5-oxa-2,8-diazaspiro[3.5]nonan-2-yl, (hydroxyC₁₋₆alkyl)piperazin-1-yl, piperazin-1-yl or piperazin-2-ylC₁₋₆alkoxy.

A further embodiment of present invention is (vi) a compound of formula (I) or (Ia), or a pharmaceutically acceptable salt thereof, according to any one of (i) to (v), wherein R⁴ is 3-amino-4-methoxy-pyrrolidin-1-yl, 4-amino-3-hydroxy-pyrrolidin-1-yl, 4-(3-aminoazetidin-1-yl)-1-piperidinyl, 5-oxa-2,8-diazaspiro[3.5]nonan-2-yl, 3-(hydroxymethyl)piperazin-1-yl, piperazin-1-yl or piperazin-2-ylmethoxy.

A further embodiment of present invention is (vii) a compound of formula (I) or (Ia), or a pharmaceutically acceptable salt thereof, according to any one of (i) to (vi), wherein R⁴ is 3-amino-4-C₁₋₆alkoxy-pyrrolidin-1-yl or piperazin-2-ylC₁₋₆alkoxy.

A further embodiment of present invention is (viii) a compound of formula (I) or (Ia), or a pharmaceutically acceptable salt thereof, according to any one of (i) to (vii), wherein R⁴ is 3-amino-4-methoxy-pyrrolidin-1-yl or piperazin-2-ylmethoxy.

A further embodiment of present invention is (ix) a compound of formula (I) or (Ia), or a pharmaceutically acceptable salt thereof, according to any one of (i) to (viii), wherein R^{3b} is H.

A further embodiment of present invention is (x) a compound of formula (I) or (Ia), or a pharmaceutically acceptable salt thereof, according to any one of (i) to (ix), wherein R^{3a} is C₁₋₆alkyl.

A further embodiment of present invention is (xi) a compound of formula (I) or (Ia), or a pharmaceutically acceptable salt thereof, according to any one of (i) to (x), wherein R^{3a} is methyl.

A further embodiment of present invention is (xii) a compound of formula (I) or (Ia), or a pharmaceutically acceptable salt thereof, according to any one of (i) to (xi), wherein R² is methyl.

A further embodiment of present invention is (xiii) a compound of formula (I) or (Ia), according to any one of (i) to (xii), wherein
R¹ is H or C₁₋₆alkyl;
R² is C₁₋₆alkyl;
R^{3a} is C₁₋₆alkyl;
R^{3b} is H;
R⁴ is 3-amino-4-C₁₋₆alkoxy-pyrrolidin-1-yl, 4-amino-3-hydroxy-pyrrolidin-1-yl, 3-aminoazetidin-1-yl-1-piperidinyl, 4-amino-3-C₁₋₆alkoxy-1-piperidinyl, 5-oxa-2,8-diazaspiro[3.5]nonan-2-yl, (hydroxyC₁₋₆alkyl)piperazin-1-yl, piperazin-1-yl or piperazin-2-ylC₁₋₆alkoxy;
Q is CH or N;
Z is CH or N;
Y is CH;
with the proviso that Q and Z are not N simultaneously;
or a pharmaceutically acceptable salt thereof.

A further embodiment of present invention is (xiv) a compound of formula (I) or (Ia), according to any one of (i) to (xiii), wherein
R¹ is H or methyl;
R² is methyl;
R^{3a} is methyl;
R^{3b} is H;
R⁴ is 3-amino-4-methoxy-pyrrolidin-1-yl, 4-amino-3-hydroxy-pyrrolidin-1-yl, 4-(3-aminoazetidin-1-yl)-1-piperidinyl, 5-oxa-2,8-diazaspiro[3.5]nonan-2-yl, 3-(hydroxymethyl)piperazin-1-yl, piperazin-1-yl or piperazin-2-ylmethoxy;
Q is CH or N;
Z is CH or N;
Y is CH;
with the proviso that Q and Z are not N simultaneously;
or a pharmaceutically acceptable salt thereof.

A further embodiment of present invention is (xv) a compound of formula (I) or (Ia), according to any one of (i) to (xiv), wherein
R¹ is H or C₁₋₆alkyl;
R² is C₁₋₆alkyl;
R^{3a} is C₁₋₆alkyl;
R^{3b} is H;
R⁴ is 3-amino-4-C₁₋₆alkoxy-pyrrolidin-1-yl or piperazin-2-ylC₁₋₆alkoxy;
Q is CH or N;
Z is CH;
Y is CH;
or a pharmaceutically acceptable salt thereof.

A further embodiment of present invention is (xvi) a compound of formula (I) or (Ia), according to any one of (i) to (xv), wherein
R¹ is H or methyl;
R² is methyl;
R^{3a} is methyl;
R^{3b} is H;
R⁴ is 3-amino-4-methoxy-pyrrolidin-1-yl or piperazin-2-ylmethoxy;
Q is CH or N;
Z is CH;
Y is CH;
or a pharmaceutically acceptable salt thereof.

The present invention relates to (i′) a compound of formula (I),

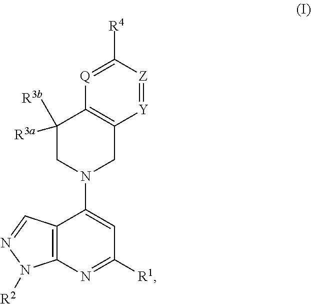

wherein
R¹ is H or C₁₋₆alkyl;
R² is C₁₋₆alkyl;
R^{3a} is H or C₁₋₆alkyl;
R^{3b} is H or C₁₋₆alkyl;
R⁴ is (C₁₋₆alkylpiperazinyl)C₁₋₆alkoxy,
   (C₁₋₆alkyl)₂piperazin-4-iumyl,
   (formylpiperazinyl)C₁₋₆alkoxy,
   1,2,3,4,6,7,8,8a-octahydropyrrolo[1,2-a]pyrazinyloxy,
   2-oxo-1-oxa-3,7-diazaspiro[4.4]nonanyl,
   2-oxo-1-oxa-3,8-diazaspiro[4.5]decanyl,
   3,8-diazabicyclo[3.2.1]octanyl,
   3,9-diazaspiro[5.5]undecanyl,
   3-oxo-1,5,6,7,8,8a-hexahydroimidazo[1,5-a]pyrazinyl,
   3-oxo-2,7-diazaspiro[4.4]nonanyl,
   3-oxo-2,8-diazaspiro[4.5]decanyl,
   3-oxo-3a,4,5,6,7,7a-hexahydro-1H-pyrrolo[3,4-c]pyridinyl,
   4-oxo-1,2,3,3a,6,6a-hexahydropyrrolo[3,4-c]pyrrolyl,
   4-piperidinylazetidinyloxy,
   5-oxa-2,8-diazaspiro[3.5]nonanyl,
   7-oxo-2,6-diazaspiro[3.4]octanyl,
   piperazinyl, which is unsubstituted or substituted by (C₁₋₆alkyl)₂aminoC₁₋₆alkyl, 1,2,3,4-tetrahydro-2,6-naphthyridinyl, C₁₋₆alkyl, C₁₋₆alkylimidazolyl, hydroxyC₁₋₆alkyl, morpholinylcarbonyl, piperidinylcarbonyl, pyrrolidinylcarbonyl or pyrrolidinylsulfonyl,
   piperazinylC₁₋₆alkoxy,
   piperidinyl, which is once or twice substituted by substituents independently selected from amino(C₁₋₆alkoxy)piperidinyl, amino(C₁₋₆alkoxy)pyrrolidinyl, aminoazetidinyl, hydroxy, ((C₁₋₆alkyl)₂amino)C₁₋₆alkyl and piperazinyl,
   pyrrolidinyl, which is twice or three times substituted by substituents independently selected from amino, hydroxy, C₁₋₆alkyl and C₁₋₆alkoxy;
Q, Z and Y are independently selected from CH and N;
or a pharmaceutically acceptable salt thereof.

Another embodiment of present invention is (ii′) a compound of formula (Ia),

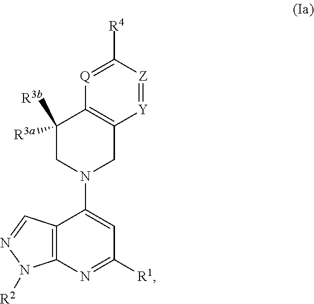

wherein
R¹ is H or C₁₋₆alkyl;
R² is C₁₋₆alkyl;
R^{3a} is H or C₁₋₆alkyl;
R^{3b} is H or C₁₋₆alkyl;
R⁴ is (C₁₋₆alkylpiperazinyl)C₁₋₆alkoxy,
   (C₁₋₆alkyl)₂piperazin-4-iumyl,
   (formylpiperazinyl)C₁₋₆alkoxy,
   1,2,3,4,6,7,8,8a-octahydropyrrolo[1,2-a]pyrazinyloxy,
   2-oxo-1-oxa-3,7-diazaspiro[4.4]nonanyl,
   2-oxo-1-oxa-3,8-diazaspiro[4.5]decanyl,
   3,8-diazabicyclo[3.2.1]octanyl,
   3,9-diazaspiro[5.5]undecanyl,
   3-oxo-1,5,6,7,8,8a-hexahydroimidazo[1,5-a]pyrazinyl,
   3-oxo-2,7-diazaspiro[4.4]nonanyl,
   3-oxo-2,8-diazaspiro[4.5]decanyl,
   3-oxo-3a,4,5,6,7,7a-hexahydro-1H-pyrrolo[3,4-c]pyridinyl,
   4-oxo-1,2,3,3a,6,6a-hexahydropyrrolo[3,4-c]pyrrolyl,
   4-piperidinylazetidinyloxy,
   5-oxa-2,8-diazaspiro[3.5]nonanyl,
   7-oxo-2,6-diazaspiro[3.4]octanyl,
   piperazinyl, which is unsubstituted or substituted by (C₁₋₆alkyl)₂aminoC₁₋₆alkyl, 1,2,3,4-tetrahydro-2,6-naphthyridinyl, C₁₋₆alkyl, C₁₋₆alkylimidazolyl, hydroxyC₁₋₆alkyl, morpholinylcarbonyl, piperidinylcarbonyl, pyrrolidinylcarbonyl or pyrrolidinylsulfonyl,
   piperazinylC₁₋₆alkoxy,
   piperidinyl, which is once or twice substituted by substituents independently selected from amino(C₁₋₆alkoxy)piperidinyl, amino(C₁₋₆alkoxy)pyrrolidinyl, aminoazetidinyl, hydroxy, ((C₁₋₆alkyl)₂amino)C₁₋₆alkyl and piperazinyl,
   pyrrolidinyl, which is twice or three times substituted by substituents independently selected from amino, hydroxy, C₁₋₆alkyl and C₁₋₆alkoxy;
Q, Z and Y are independently selected from CH and N;
or a pharmaceutically acceptable salt thereof.

A further embodiment of present invention is (iii′) a compound of formula (I) or (Ia) according to (i′) or (ii′), or a pharmaceutically acceptable salt thereof, wherein Q is CH, Z is CH and Y is CH.

A further embodiment of present invention is (iv′) a compound of formula (I) or (Ia), according to any one of (i′) to (iii′), or a pharmaceutically acceptable salt thereof, wherein R⁴ is (1-C₁₋₆alkylpiperazin-2-yl)C₁₋₆alkoxy, (C₁₋₆alkyl)₂piperazin-4-ium-1-yl, (1-formylpiperazin-2-yl)C₁₋₆alkoxy, 1,2,3,4,6,7,8,8a-octahydropyrrolo[1,2-a]pyrazin-7-yloxy, 2-oxo-1-oxa-3,7-diazaspiro[4.4]nonan-3-yl, 2-oxo-1-oxa-3,8-diazaspiro[4.5]decan-3-yl, 3,8-diazabicyclo[3.2.1]octan-8-yl, 3,9-diazaspiro[5.5]undecan-3-yl, 3-oxo-1,5,6,7,8,8a-hexahydroimidazo[1,5-a]pyrazin-2-yl, 3-oxo-2,7-diazaspiro[4.4]nonan-2-yl, 3-oxo-2,8-diazaspiro[4.5]decan-2-yl, 3-oxo-3a,4,5,6,7,7a-hexahydro-1H-pyrrolo[3,4-c]pyridin-2-yl, 4-oxo-1,2,3,3a,6,6a-hexahydropyrrolo[3,4-c]pyrrol-5-yl, 4-piperidylazetidin-3-yloxy, 5-oxa-2,8-diazaspiro[3.5]nonan-2-yl, 7-oxo-2,6-diazaspiro[3.4]octan-6-yl, piperazin-1-yl, 1,2,3,4-tetrahydro-2,6-naphthyridin-7-yl-piperazin-1-yl, 1-C₁₋₆alkylimidazol-4-yl-piperazin-1-yl, (hydroxyC₁₋₆alkyl)piperazin-1-yl, C₁₋₆alkyl-piperazin-1-yl, (morpholin-2-ylcarbonyl)piperazin-1-yl, (C₁₋₆alkyl)₂aminoC₁₋₆alkyl-piperazin-1-yl, piperidin-4-ylcarbonyl-piperazin-1-yl, pyrrolidin-2-ylcarbonyl-piperazin-1-yl, pyrrolidin-3-ylsulfonyl-piperazin-1-yl, piperazin-1-ylC₁₋₆alkoxy, piperazin-2-ylC₁₋₆alkoxy, (3-amino-4-C₁₋₆alkoxy-pyrrolidin-1-yl)-1-piperidinyl, 3-aminoazetidin-1-yl-1-piperidinyl, (4-amino-3-hydroxy-1-piperidinyl)-1-piperidinyl, 4-hydroxy-4-((C₁₋₆alkyl)₂amino)C₁₋₆alkyl-1-piperidinyl, piperazin-1-yl-1-piperidinyl, 3-amino-4-C₁₋₆alkoxy-pyrrolidin-1-yl, 3-amino-4-hydroxy-pyrrolidin-1-yl or 4-amino-3-hydroxy-3-C₁₋₆alkyl-pyrrolidin-1-yl.

A further embodiment of present invention is (v′) a compound of formula (I) or (Ia) according to any one of (i′) to (iv′), wherein R⁴ is 3-amino-4-C₁₋₆alkoxy-pyrrolidin-1-yl, 3-amino-4-hydroxy-pyrrolidin-1-yl, 3-aminoazetidin-1-yl-1-piperidinyl, 5-oxa-2,8-diazaspiro[3.5]nonan-2-yl, (hydroxyC₁₋₆alkyl)piperazin-1-yl or piperazin-2-ylC₁₋₆alkoxy.

A further embodiment of present invention is (vi′) a compound of formula (I) or (Ia), or a pharmaceutically acceptable salt thereof, according to any one of (i′) to (v′), wherein R⁴ is 3-amino-4-methoxy-pyrrolidin-1-yl, 3-amino-4-hydroxy-pyrrolidin-1-yl, 4-(3-aminoazetidin-1-yl)-1-piperidinyl, 5-oxa-2,8-diazaspiro[3.5]nonan-2-yl, 3-(hydroxymethyl)piperazin-1-yl or piperazin-2-ylmethoxy.

A further embodiment of present invention is (vii′) a compound of formula (I) or (Ia), or a pharmaceutically acceptable salt thereof, according to any one of (i′) to (vi′), wherein R⁴ is 3-amino-4-C₁₋₆alkoxy-pyrrolidin-1-yl or piperazin-2-ylC₁₋₆alkoxy.

A further embodiment of present invention is (viii′) a compound of formula (I) or (Ia), or a pharmaceutically acceptable salt thereof, according to any one of (i′) to (vii′), wherein R⁴ is 3-amino-4-methoxy-pyrrolidin-1-yl or piperazin-2-ylmethoxy.

A further embodiment of present invention is (ix′) a compound of formula (I) or (Ia), or a pharmaceutically acceptable salt thereof, according to any one of (i′) to (viii′), wherein RB is H.

A further embodiment of present invention is (x′) a compound of formula (I) or (Ia), or a pharmaceutically acceptable salt thereof, according to any one of (i′) to (ix′), wherein R^{3a} is C₁₋₆alkyl.

A further embodiment of present invention is (xi′) a compound of formula (I) or (Ia), or a pharmaceutically acceptable salt thereof, according to any one of (i′) to (x′), wherein R^{3a} is methyl.

A further embodiment of present invention is (xii′) a compound of formula (I) or (Ia), or a pharmaceutically acceptable salt thereof, according to any one of (i′) to (xi′), wherein R² is methyl.

A further embodiment of present invention is (xiii′) a compound of formula (I) or (Ia), according to any one of (i′) to (xii′), wherein
R¹ is H or C₁₋₆alkyl;
R² is C₁₋₆alkyl;
R^{3a} is C₁₋₆alkyl;
R^{3b} is H;
R⁴ is 3-amino-4-C₁₋₆alkoxy-pyrrolidin-1-yl, 3-amino-4-hydroxy-pyrrolidin-1-yl, 3-aminoazetidin-1-yl-1-piperidinyl, 5-oxa-2,8-diazaspiro[3.5]nonan-2-yl, (hydroxyC₁₋₆alkyl)piperazin-1-yl or piperazin-2-ylC₁₋₆alkoxy;
Q is CH;
Z is CH;
Y is CH;
or a pharmaceutically acceptable salt thereof.

A further embodiment of present invention is (xiv′) a compound of formula (I) or (Ia), according to any one of (i′) to (xiii′), wherein
R¹ is H or methyl;
R² is methyl;
R^{3a} is methyl;
R^{3b} is H;
R⁴ is 3-amino-4-methoxy-pyrrolidin-1-yl, 3-amino-4-hydroxy-pyrrolidin-1-yl, 4-(3-aminoazetidin-1-yl)-1-piperidinyl, 5-oxa-2,8-diazaspiro[3.5]nonan-2-yl, 3-(hydroxymethyl)piperazin-1-yl or piperazin-2-ylmethoxy;
Q is CH;
Z is CH;
Y is CH;
or a pharmaceutically acceptable salt thereof.

A further embodiment of present invention is (xv′) a compound of formula (I) or (Ia), according to any one of (i′) to (xiv′), wherein
R¹ is H;
R² is C₁₋₆alkyl;
R^{3a} is C₁₋₆alkyl;
R^{3b} is H;
R⁴ is 3-amino-4-C₁₋₆alkoxy-pyrrolidin-1-yl or piperazin-2-ylC₁₋₆alkoxy;
Q is CH;
Z is CH;
Y is CH;
or a pharmaceutically acceptable salt thereof.

A further embodiment of present invention is (xvi′) a compound of formula (I) or (Ia), according to any one of (i′) to (xv′), wherein
R¹ is H;
R² is methyl;
R^{3a} is methyl;
R^{3b} is H;
R⁴ is 3-amino-4-methoxy-pyrrolidin-1-yl or piperazin-2-ylmethoxy;
Q is CH;
Z is CH;
Y is CH;
or a pharmaceutically acceptable salt thereof.

Another embodiment of present invention is a compound of formula (I) or (Ia) selected from the following:
(4R)-4-methyl-2-(1-methylpyrazolo[3,4-b]pyridin-4-yl)-6-piperazin-1-yl-3,4-dihydro-1H-isoquinoline;
(4R)-2-(1,6-dimethylpyrazolo[3,4-b]pyridin-4-yl)-4-methyl-6-piperazin-1-yl-3,4-dihydro-1H-isoquinoline;
2-(1-methylpyrazolo[3,4-b]pyridin-4-yl)-6-piperazin-1-yl-3,4-dihydro-1H-isoquinoline;
3-[4-methyl-2-(1-methylpyrazolo[3,4-b]pyridin-4-yl)-3,4-dihydro-1H-isoquinolin-6-yl]-3,9-diazaspiro[5.5]undecane;
3-[2-(1,6-dimethylpyrazolo[3,4-b]pyridin-4-yl)-4-methyl-3,4-dihydro-1H-isoquinolin-6-yl]-3,9-diazaspiro[5.5]undecane;
3-[4,4-dimethyl-2-(1-methylpyrazolo[3,4-b]pyridin-4-yl)-1,3-dihydroisoquinolin-6-yl]-3,9-diazaspiro[5.5]undecane;
4-piperidyl-[4-[(4R)-4-methyl-2-(1-methylpyrazolo[3,4-b]pyridin-4-yl)-3,4-dihydro-1H-isoauinolin-6-yl]piperazin-1-yl]methanone;
pyrrolidin-2-yl-[4-[(4R)-4-methyl-2-(1-methylpyrazolo[3,4-b]pyridin-4-yl)-3,4-dihydro-1H-isoquinolin-6-yl]piperazin-1-yl]methanone;
7-[4-[(4R)-4-methyl-2-(1-methylpyrazolo[3,4-b]pyridin-4-yl)-3,4-dihydro-1H-isoquinolin-6-yl]piperazin-1-yl]-1,2,3,4-tetrahydro-2,6-naphthyridine;
(4R)-4-methyl-2-(1-methylpyrazolo[3,4-b]pyridin-4-yl)-6-(4-piperazin-1-yl-1-piperidyl)-3,4-dihydro-1H-isoquinoline;
(3R,4R)-4-methoxy-1-[1-[(4R)-4-methyl-2-(1-methylpyrazolo[3,4-b]pyridin-4-yl)-3,4-dihydro-1H-isoquinolin-6-yl]-4-piperidyl]pyrrolidin-3-amine;
morpholin-2-yl-[4-[(4R)-4-methyl-2-(1-methylpyrazolo[3,4-b]pyridin-4-yl)-3,4-dihydro-1H-isoquinolin-6-yl]piperazin-1-yl]methanone;
4-[(dimethylamino)methyl]-1-[(4R)-4-methyl-2-(1-methylpyrazolo[3,4-b]pyridin-4-yl)-3,4-dihydro-1H-isoquinolin-6-yl]piperidin-4-ol;
4-[(dimethylamino)methyl]-1-[(4R)-2-(1,6-dimethylpyrazolo[3,4-b]pyridin-4-yl)-4-methyl-3,4-dihydro-1H-isoquinolin-6-yl]piperidin-4-ol;
(4R)-4-methyl-6-(3-methylpiperazin-1-yl)-2-(1-methylpyrazolo[3,4-b]pyridin-4-yl)-3,4-dihydro-1H-isoquinoline;
2-[(4R)-4-methyl-2-(1-methylpyrazolo[3,4-b]pyridin-4-yl)-3,4-dihydro-1H-isoquinolin-6-yl]-2,8-diazaspiro[4.5]decan-3-one;
cis-5-[(4R)-4-methyl-2-(1-methylpyrazolo[3,4-b]pyridin-4-yl)-3,4-dihydro-1H-isoquinolin-6-yl]-1,2,3,3a,6,6a-hexahydropyrrolo[3,4-c]pyrrol-4-one;
(4R)-4-methyl-2-(1-methylpyrazolo[3,4-b]pyridin-4-yl)-6-[[(2R)-piperazin-2-yl]methoxy]-3,4-dihydro-1H-isoquinoline;
(4R)-4-methyl-2-(1-methylpyrazolo[3,4-b]pyridin-4-yl)-6-[[(2S)-piperazin-2-yl]methoxy]-3,4-dihydro-1H-isoquinoline;
(3R,4R)-4-methoxy-1-[(4R)-4-methyl-2-(1-methylpyrazolo[3,4-b]pyridin-4-yl)-3,4-dihydro-1H-isoquinolin-6-yl]pyrrolidin-3-amine;
2-[(4R)-4-methyl-2-(1-methylpyrazolo[3,4-b]pyridin-4-yl)-3,4-dihydro-1H-isoquinolin-6-yl]-5-oxa-2,8-diazaspiro[3.5]nonane;
(4R)-2-(1-ethylpyrazolo[3,4-b]pyridin-4-yl)-4-methyl-6-piperazin-1-yl-3,4-dihydro-H-isoquinoline;
(3R,4R)-4-methoxy-1-[(4R)-2-(1,6-dimethylpyrazolo[3,4-b]pyridin-4-yl)-4-methyl-3,4-dihydro-1H-isoquinolin-6-yl]pyrrolidin-3-amine;
(8aR)-2-[(4R)-4-methyl-2-(1-methylpyrazolo[3,4-b]pyridin-4-yl)-3,4-dihydro-1H-isoquinolin-6-yl]-1,5,6,7,8,8a-hexahydroimidazo[1,5-a]pyrazin-3-one;
(8aS)-2-[(4R)-4-methyl-2-(1-methylpyrazolo[3,4-b]pyridin-4-yl)-3,4-dihydro-1H-isoquinolin-6-yl]-1,5,6,7,8,8a-hexahydroimidazo[1,5-a]pyrazin-3-one;
3-[(4R)-4-methyl-2-(1-methylpyrazolo[3,4-b]pyridin-4-yl)-3,4-dihydro-1H-isoquinolin-6-yl]-1-oxa-3,8-diazaspiro[4.5]decan-2-one;
(4R)-6-(3,8-diazabicyclo[3.2.1]octan-8-yl)-4-methyl-2-(1-methylpyrazolo[3,4-b]pyridin-4-yl)-3,4-dihydro-1H-isoquinoline;
1-[1-[(4R)-4-methyl-2-(1-methylpyrazolo[3,4-b]pyridin-4-yl)-3,4-dihydro-1H-isoquinolin-6-yl]-4-piperidyl]azetidin-3-amine;
trans-4-amino-1-[1-[(4R)-4-methyl-2-(1-methylpyrazolo[3,4-b]pyridin-4-yl)-3,4-dihydro-1H-isoquinolin-6-yl]-4-piperidyl]piperidin-3-ol;
(4R)-2-(1,6-dimethylpyrazolo[3,4-b]pyridin-4-yl)-4-methyl-6-[[(2R)-piperazin-2-yl]methoxy]-3,4-dihydro-1H-isoquinoline;
(4R)-2-(1,6-dimethylpyrazolo[3,4-b]pyridin-4-yl)-4-methyl-6-[[(2S)-piperazin-2-yl]methoxy]-3,4-dihydro-1H-isoquinoline;
(4R)-4-methyl-2-(1-methylpyrazolo[3,4-b]pyridin-4-yl)-6-(4-pyrrolidin-3-ylsulfonylpiperazin-1-yl)-3,4-dihydro-1H-isoquinoline;
6-[(4R)-4-methyl-2-(1-methylpyrazolo[3,4-b]pyridin-4-yl)-3,4-dihydro-1H-isoquinolin-6-yl]-2,6-diazaspiro[3.4]octan-7-one;
3-[(4R)-4-methyl-2-(1-methylpyrazolo[3,4-b]pyridin-4-yl)-3,4-dihydro-1H-isoquinolin-6-yl]-1-oxa-3,7-diazaspiro[4.4]nonan-2-one;
(3R,4R)-4-amino-3-methyl-1-[(4R)-4-methyl-2-(1-methylpyrazolo[3,4-b]pyridin-4-yl)-3,4-dihydro-1H-isoquinolin-6-yl]pyrrolidin-3-ol;
(4R)-4-methyl-6-[4-[(1-methylimidazol-4-yl)methyl]piperazin-1-yl]-2-(1-methylpyrazolo[3,4-b]pyridin-4-yl)-3,4-dihydro-1H-isoquinoline;
N,N-dimethyl-2-[4-[(4R)-4-methyl-2-(1-methylpyrazolo[3,4-b]pyridin-4-yl)-3,4-dihydro-1H-isoquinolin-6-yl]piperazin-1-yl]ethanamine;
(4R)-6-(4,4-dimethylpiperazin-4-ium-1-yl)-4-methyl-2-(1-methylpyrazolo[3,4-b]pyridin-4-yl)-3,4-dihydro-1H-isoquinoline chloride;
(4R)-4-methyl-2-(1-methylpyrazolo[3,4-b]pyridin-4-yl)-6-(2-piperazin-1-ylethoxy)-3,4-dihydro-1H-isoquinoline;
(4R)-4-methyl-2-(1-methylpyrazolo[3,4-b]pyridin-4-yl)-6-[1-(4-piperidyl)azetidin-3-yl]oxy-3,4-dihydro-1H-isoquinoline;
(4R)-4-methyl-2-(1-methylpyrazolo[3,4-b]pyridin-4-yl)-6-[[(7R,8aS)-1,2,3,4,6,7,8,8a-octahydropyrrolo[1,2-a]pyrazin-7-yl]oxy]-3,4-dihydro-1H-isoquinoline;
(4R)-4-methyl-2-(1-methylpyrazolo[3,4-b]pyridin-4-yl)-6-[[(2R)-1-methylpiperazin-2-yl]methoxy]-3,4-dihydro-1H-isoquinoline;
(4R)-4-methyl-2-(1-methylpyrazolo[3,4-b]pyridin-4-yl)-6-[[(2S)-1-methylpiperazin-2-yl]methoxy]-3,4-dihydro-1H-isoquinoline;
(2S)-2-[[(4R)-4-methyl-2-(1-methylpyrazolo[3,4-b]pyridin-4-yl)-3,4-dihydro-1H-isoquinolin-6-yl]oxymethyl]piperazine-1-carbaldehyde;
trans-4-amino-1-[(4R)-4-methyl-2-(1-methylpyrazolo[3,4-b]pyridin-4-yl)-3,4-dihydro-1H-isoquinolin-6-yl]pyrrolidin-3-ol;
trans-4-amino-1-[(4R)-2-(1,6-dimethylpyrazolo[3,4-b]pyridin-4-yl)-4-methyl-3,4-dihydro-1H-isoquinolin-6-yl]pyrrolidin-3-ol;
2-[(4R)-2-(1,6-dimethylpyrazolo[3,4-b]pyridin-4-yl)-4-methyl-3,4-dihydro-1H-isoquinolin-6-yl]-5-oxa-2,8-diazaspiro[3.5]nonane;
[4-[(4R)-4-methyl-2-(1-methylpyrazolo[3,4-b]pyridin-4-yl)-3,4-dihydro-1H-isoquinolin-6-yl]piperazin-2-yl]methanol;
[(2R)-4-[(4R)-4-methyl-2-(1-methylpyrazolo[3,4-b]pyridin-4-yl)-3,4-dihydro-H-isoquinolin-6-yl]piperazin-2-yl]methanol;
[(2S)-4-[(4R)-4-methyl-2-(1-methylpyrazolo[3,4-b]pyridin-4-yl)-3,4-dihydro-H-isoquinolin-6-yl]piperazin-2-yl]methanol;
2-[(4R)-4-methyl-2-(1-methylpyrazolo[3,4-b]pyridin-4-yl)-3,4-dihydro-1H-isoquinolin-6-yl]-2,7-diazaspiro[4.4]nonan-3-one;
(5S)-2-[(4R)-4-methyl-2-(1-methylpyrazolo[3,4-b]pyridin-4-yl)-3,4-dihydro-1H-isoquinolin-6-yl]-2,7-diazaspiro[4.4]nonan-3-one;
(5R)-2-[(4R)-4-methyl-2-(1-methylpyrazolo[3,4-b]pyridin-4-yl)-3,4-dihydro-1H-isoquinolin-6-yl]-2,7-diazaspiro[4.4]nonan-3-one;
cis-2-[(4R)-4-methyl-2-(1-methylpyrazolo[3,4-b]pyridin-4-yl)-3,4-dihydro-1H-isoquinolin-6-yl]-3a,4,5,6,7,7a-hexahydro-1H-pyrrolo[3,4-c]pyridin-3-one;
(3aS,7aS)-2-[(4R)-4-methyl-2-(1-methylpyrazolo[3,4-b]pyridin-4-yl)-3,4-dihydro-H-isoquinolin-6-yl]-3a,4,5,6,7,7a-hexahydro-1H-pyrrolo[3,4-c]pyridin-3-one;
(3aR,7aR)-2-[(4R)-4-methyl-2-(1-methylpyrazolo[3,4-b]pyridin-4-yl)-3,4-dihydro-1H-isoquinolin-6-yl]-3a,4,5,6,7,7a-hexahydro-1H-pyrrolo[3,4-c]pyridin-3-one;
1-[(8S)-6-(1,6-dimethylpyrazolo[3,4-b]pyridin-4-yl)-8-methyl-7,8-dihydro-5H-1,6-naphthyridin-2-yl]piperidin-4-amine;
4-methyl-1-[(8S)-6-(1,6-dimethylpyrazolo[3,4-b]pyridin-4-yl)-8-methyl-7,8-dihydro-5H-1,6-naphthyridin-2-yl]piperidin-4-amine;
N,N-dimethyl-1-[(8S)-6-(1,6-dimethylpyrazolo[3,4-b]pyridin-4-yl)-8-methyl-7,8-dihydro-5H-1,6-naphthyridin-2-yl]piperidin-4-amine;
4-[(dimethylamino)methyl]-1-[(8S)-6-(1,6-dimethylpyrazolo[3,4-b]pyridin-4-yl)-8-methyl-7,8-dihydro-5H-1,6-naphthyridin-2-yl]piperidin-4-ol;
(8S)-8-methyl-6-(1-methylpyrazolo[3,4-b]pyridin-4-yl)-2-piperazin-1-yl-7,8-dihydro-5H-1,6-naphthyridine;
(3R,4R)-4-methoxy-1-[(8S)-6-(1,6-dimethylpyrazolo[3,4-b]pyridin-4-yl)-8-methyl-7,8-dihydro-5H-1,6-naphthyridin-2-yl]pyrrolidin-3-amine;
(8S)-6-(1,6-dimethylpyrazolo[3,4-b]pyridin-4-yl)-8-methyl-2-(4-piperidyloxy)-7,8-dihydro-5H-1,6-naphthyridine;
(3S,4S)-3-methoxy-1-[(8S)-6-(1,6-dimethylpyrazolo[3,4-b]pyridin-4-yl)-8-methyl-7,8-dihydro-5H-1,6-naphthyridin-2-yl]piperidin-4-amine;
(8S)-6-(1,6-dimethylpyrazolo[3,4-b]pyridin-4-yl)-8-methyl-2-piperazin-1-yl-7,8-dihydro-5H-1,6-naphthyridine;
(8S)-6-(1,6-dimethylpyrazolo[3,4-b]pyridin-4-yl)-8-methyl-2-(4-methylpiperazin-1-yl)-7,8-dihydro-5H-1,6-naphthyridine;
2-[(8S)-6-(1,6-dimethylpyrazolo[3,4-b]pyridin-4-yl)-8-methyl-7,8-dihydro-5H-1,6-naphthyridin-2-yl]-5-oxa-2,8-diazaspiro[3.5]nonane;
(3S,4R)-4-fluoro-1-[(8S)-6-(1,6-dimethylpyrazolo[3,4-b]pyridin-4-yl)-8-methyl-7,8-dihydro-5H-1,6-naphthyridin-2-yl]pyrrolidin-3-amine;
8-[(8S)-6-(1,6-dimethylpyrazolo[3,4-b]pyridin-4-yl)-8-methyl-7,8-dihydro-5H-1,6-naphthyridin-2-yl]-5-oxa-2,8-diazaspiro[3.5]nonane;
(8R)-6-(1,6-dimethylpyrazolo[3,4-b]pyridin-4-yl)-8-methyl-2-piperazin-1-yl-7,8-dihydro-5H-1,6-naphthyridine;
(3S,4S)-3-methoxy-1-[(5R)-7-(1,6-dimethylpyrazolo[3,4-b]pyridin-4-yl)-5-methyl-6,8-dihydro-5H-2,7-naphthyridin-3-yl]piperidin-4-amine;
(4R)-2-(1,6-dimethylpyrazolo[3,4-b]pyridin-4-yl)-4-methyl-6-piperazin-1-yl-3,4-dihydro-1H-2,7-naphthyridine;
(4R)-6-(4,7-diazaspiro[2.5]octan-7-yl)-2-(1,6-dimethylpyrazolo[3,4-b]pyridin-4-yl)-4-methyl-3,4-dihydro-1H-2,7-naphthyridine;
(4R)-2-(1,6-dimethylpyrazolo[3,4-b]pyridin-4-yl)-4-methyl-6-[[(7R,8aS)-1,2,3,4,6,7,8,8a-octahydropyrrolo[1,2-a]pyrazin-7-yl]oxy]-3,4-dihydro-1H-2,7-naphthyridine;
(4R)-2-(1,6-dimethylpyrazolo[3,4-b]pyridin-4-yl)-4-methyl-6-(2-piperazin-1-ylethoxy)-3,4-dihydro-1H-2,7-naphthyridine;
(3R,4R)-4-methoxy-1-[(5R)-7-(1,6-dimethylpyrazolo[3,4-b]pyridin-4-yl)-5-methyl-6,8-dihydro-5H-2,7-naphthyridin-3-yl]pyrrolidin-3-amine;
2-[(5R)-7-(1,6-dimethylpyrazolo[3,4-b]pyridin-4-yl)-5-methyl-6,8-dihydro-5H-2,7-naphthyridin-3-yl]-5-oxa-2,8-diazaspiro[3.5]nonane;
(3R,4R)-3-fluoro-1-[(5R)-7-(1,6-dimethylpyrazolo[3,4-b]pyridin-4-yl)-5-methyl-6,8-dihydro-5H-2,7-naphthyridin-3-yl]piperidin-4-amine; and
(4R)-2-(1,6-dimethylpyrazolo[3,4-b]pyridin-4-yl)-4-methyl-6-[(3R)-3-(methoxymethyl)piperazin-1-yl]-3,4-dihydro-1H-2,7-naphthyridine;

or a pharmaceutically acceptable salt thereof.

### Synthesis

The compounds of the present invention can be prepared by any conventional means. Suitable processes for synthesizing these compounds as well as their starting materials are provided in the schemes below and in the examples. All substituents, in particular, R¹, R², R^{3a}, R^{3b}, R⁴, Q, Z and Y are as defined above unless otherwise indicated. Furthermore, and unless explicitly otherwise stated, all reactions, reaction conditions, abbreviations and symbols have the meanings well known to a person of ordinary skill in organic chemistry.

General synthetic routes for preparing the compound of formula (I) or (Ia) are shown below.

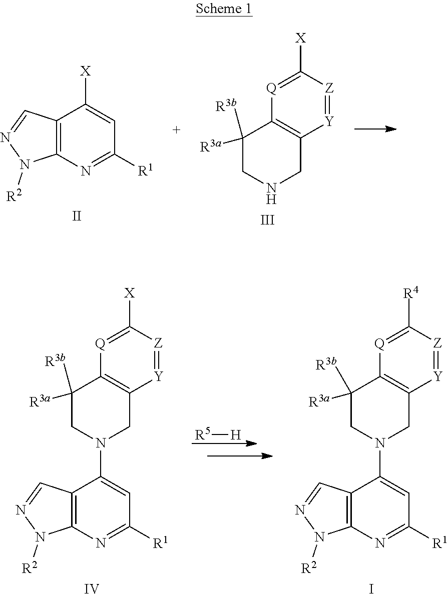

Wherein X is halogen; R⁵ is R⁴ or R⁴ with protection group, wherein the protection group can be selected from Boc, Cbz, acyl, sulfonyl, alkyl or aryl.

The coupling of compound of formula (II) with (III) can be achieved by direct coupling at elevated temperature in the presence of a base, such as DIPEA or CsF to provide compound of formula (IV). Subsequently, the coupling of compound of formula (IV) with R⁴—H can be achieved by direct coupling under Buchwald-Hartwig C—N or C—O bond formation conditions (ref: Acc. Chem. Res. 1998, 31, 805-818; Chem. Rev. 2016, 116, 12564-12649; Topics in Current Chemistry, 2002, 219, 131-209; and references cited therein) with a catalyst, such as RuPhos Pd G2, [Pd(allyl)Cl]₂/JackiePhos, Pd[P(o-tol)₃]₂/CyPF-t-Bu and a base, such as Cs₂CO₃ or t-BuONa, to provide compound of formula (I). In some embodiments, the coupling of compound of formula (IV) and R⁵—H may give a product containing a protecting group, e.g. Boc or Cbz, originated from R⁵—H, which will be removed before affording the final compound of formula (I). And in some other embodiments, the compound of formula (I) with a terminal secondary amine is further introduced with acyl group, sulfonyl group, alkyl group or aryl group to afford the final compound of formula (I).

Compounds of this invention can be obtained as mixtures of diastereomers or enantiomers, which can be separated by methods well known in the art, e.g. (chiral) HPLC or SFC.

This invention also relates to a process for the preparation of a compound of formula (I) or (Ia) comprising the following step:
a) the Buchwald-Hartwig C—N or C—O bond formation of compound of formula (IV),

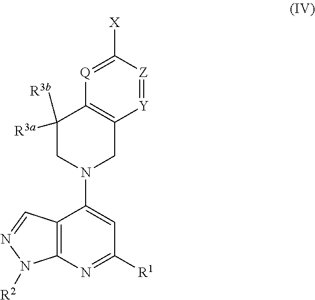

with R⁵—H, in the presence of a catalyst and a base;
wherein
in step a), the catalyst can be, for example, RuPhos Pd G2, [Pd(allyl)Cl]₂/JackiePhos, Pd[P(o-tol)₃]₂/CyPF-t-Bu; the base can be, for example, Cs₂CO₃ or t-BuONa;

A compound of formula (I) or (Ia) when manufactured according to the above process is also an object of the invention.

### Indications and Methods of Treatment

The present invention provides compounds that can be used as TLR7 and/or TLR8 and/or TLR9 antagonist, which inhibits pathway activation through TLR7 and/or TLR8 and/or TLR9 as well as respective downstream biological events including, but not limited to, innate and adaptive immune responses mediated through the production of all types of cytokines and all forms of auto-antibodies. Accordingly, the compounds of the invention are useful for blocking TLR7 and/or TLR8 and/or TLR9 in all types of cells that express such receptor(s) including, but not limited to, plasmacytoid dendritic cell, B cell, T cell, macrophage, monocyte, neutrophil, keratinocyte, epithelial cell. As such, the compounds can be used as a therapeutic or prophylactic agent for systemic lupus erythematosus and lupus nephritis.

The present invention provides methods for treatment or prophylaxis of systemic lupus erythematosus and lupus nephritis in a patient in need thereof.

Another embodiment includes a method of treating or preventing systemic lupus erythematosus and lupus nephritis in a mammal in need of such treatment, wherein the method comprises administering to said mammal a therapeutically effective amount of a compound of formula (I), a stereoisomer, tautomer, prodrug or pharmaceutically acceptable salt thereof.

### BRIEF DESCRIPTION OF THE FIGURE

FIG. 1 co-crystal structure of Example 1 with protein TLR8 (only the structure of Example 1 was shown), which used to determine the chiral configuration of Example 1 and Intermediate A.

### EXAMPLES

The invention will be more fully understood by reference to the following examples. They should not, however, be construed as limiting the scope of the invention.

### Abbreviations

The invention will be more fully understood by reference to the following examples. They should not, however, be construed as limiting the scope of the invention.

Abbreviations used herein are as follows:
ACN: acetonitrile
BINAP: (1,1′-binaphthalene-2,2′-diyl)bis(diphenylphosphine)
Boc₂O: di-tert butyl dicarbonate
CbzCl: benzylchloroformate
CyPF-t-Bu: [(R)-1-[(S)-2-(dicyclohexylphosphino)ferrocenyl]ethyl]di-tert-butylphosphine
DCE: dichloroethane
DEA: diethylamine
DIPEA: N,N-diisopropylethylamine
DMA: dimethylacetamide
DMFDMA: N,N-dimethylformamide dimethyl acetal
DMSO: Dimethyl sulfoxide
EDTA: Ethylenediaminetetraacetic acid
EtOAc or EA: ethyl acetate
FA: formic acid
HLM human liver microsome
IC₅₀: half inhibition concentration
JackiePhos: Bis(3,5-bis(trifluoromethyl)phenyl)(2′,4′,6′-triisopropyl-3,6-dimethoxybiphenyl-2-yl)phosphine
   LCMS liquid chromatography-mass spectrometry
   DMEM: Dulbecco's Modified Eagle's Medium
   MS: mass spectrometry
   [Pd(allyl)Cl]₂: allylpalladium(II) chloride dimer
   Pd[P(o-tol)₃]₂: bis(tri-o-tolylphosphine)palladium
   PE: petroleum ether
   prep-HPLC: preparative high performance liquid chromatography
   rt: room temperature
   RuPhos Pd G2: chloro(2-dicyclohexylphosphino-2′,6′-diisopropoxy-1,1′-biphenyl)[2-(2′-amino-1,1′-biphenyl)]palladium(II) 2nd generation
   SFC: supercritical fluid chromatography
   TEA: trimethylamine
   TFA: trifluoroacetic acid
   Tf₂O: trifluoromethanesulfonic anhydride
   v/v: volume ratio

### General Experimental Conditions

Intermediates and final compounds were purified by flash chromatography using one of the following instruments: i) Biotage SP1 system and the Quad 12/25 Cartridge module. ii) ISCO combi-flash chromatography instrument. Silica gel brand and pore size: i) KP-SIL 60 Å, particle size: 40-60 μm; ii) CAS registry NO: Silica Gel: 63231-67-4, particle size: 47-60 micron silica gel; iii) ZCX from Qingdao Haiyang Chemical Co., Ltd, pore: 200-300 or 300-400.

Intermediates and final compounds were purified by preparative HPLC on reversed phase column using XBridge™ Prep-C18 (5 μm, OBD™ 30×100 mm) column, SunFire™ Prep-C18 (5 μm, OBD™ 30×100 mm) column, Phenomenex Synergi-C18 (10 μm, 25×150 mm) or Phenomenex Gemini-C18 (10 μm, 25×150 mm). Waters AutoP purification System (Sample Manager 2767, Pump 2525, Detector: Micromass ZQ and UV 2487, solvent system: acetonitrile and 0.10% ammonium hydroxide in water; acetonitrile and 0.10% FA in water or acetonitrile and 0.1% TFA in water). Or Gilson-281 purification System (Pump 322, Detector: UV 156, solvent system: acetonitrile and 0.05% ammonium hydroxide in water; acetonitrile and 0.225% FA in water; acetonitrile and 0.05% HCl in water; acetonitrile and 0.075% TFA in water; or acetonitrile and water).

For SFC chiral separation, intermediates were separated by chiral column (Daicel chiralpak IC, 5 μm, 30×250 mm), AS (10 μm, 30×250 mm) or AD (10 μm, 30×250 mm) using Mettler Toledo Multigram III system SFC, Waters 80Q preparative SFC or Thar 80 preparative SFC, solvent system: CO₂ and IPA (0.5% TEA in IPA) or CO₂ and MeOH (0.1% NH₃·H₂O in MeOH), back pressure 100bar, detection UV@ 254 or 220 nm.
LC/MS spectra of compounds were obtained using a LC/MS (Waters™ Alliance 2795-Micromass ZQ, Shimadzu Alliance 2020-Micromass ZQ or Agilent Alliance 6110-Micromass ZQ), LC/MS conditions were as follows (running time 3 or 1.5 mins):
Acidic condition I: A: 0.1% TFA in H₂O; B: 0.1% TFA in acetonitrile;
Acidic condition II: A: 0.0375% TFA in H₂O; B: 0.01875% TFA in acetonitrile;
Basic condition I: A: 0.1% NH₃·H₂O in H₂O; B: acetonitrile;
Basic condition II: A: 0.025% NH₃·H₂O in H₂O; B: acetonitrile;
Neutral condition: A: H₂O; B: acetonitrile.

Mass spectra (MS): generally only ions which indicate the parent mass are reported, and unless otherwise stated the mass ion quoted is the positive mass ion (MH)*.

NMR Spectra were obtained using Bruker Avance 400 MHz.

The microwave assisted reactions were carried out in a Biotage Initiator Sixty microwave synthesizer. All reactions involving air-sensitive reagents were performed under an argon or nitrogen atmosphere. Reagents were used as received from commercial suppliers without further purification unless otherwise noted.

### PREPARATIVE EXAMPLES

The following examples are intended to illustrate the meaning of the present invention but should by no means represent a limitation within the meaning of the present invention:

### Intermediate A

### (4R)-6-chloro-4-methyl-1,2,3,4-tetrahydroisoquinoline

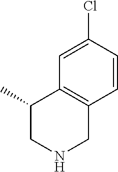

### Intermediate B

### (4S)-6-chloro-4-methyl-1,2,3,4-tetrahydroisoquinoline

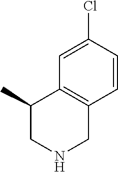

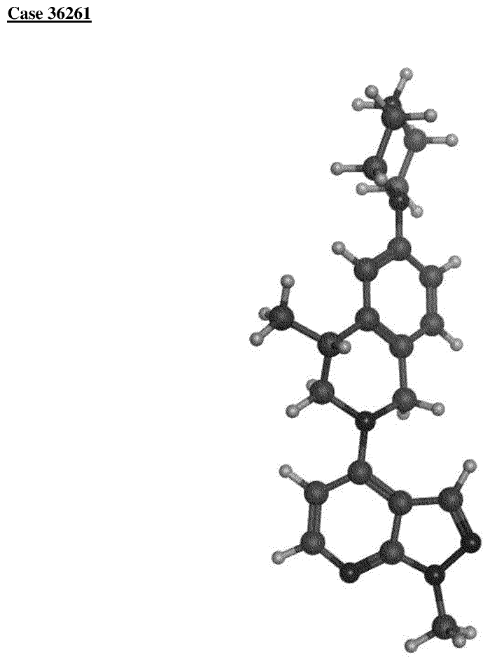

The title compounds were separated from 6-chloro-4-methyl-1,2,3,4-tetrahydroisoquinoline (the mixture of Intermediate A and B, CAS: 73037-81-7, Vendor: Bepharm) by chiral SFC (Gradient: 20% Isopropanol (0.1% NH₃H₂O) in CO₂, Column: IG, 250×20 mm I.D., 5 μm). The configuration of Intermediate A was confirmed by co-crystal structure of Example 1 with protein TLR8 (FIG. 1).

### Intermediate C

### (8S)-2-chloro-8-methyl-5,6,7,8-tetrahydro-1,6-naphthyridine

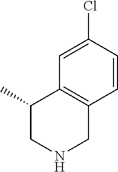

The title compound was prepared according to the following scheme:

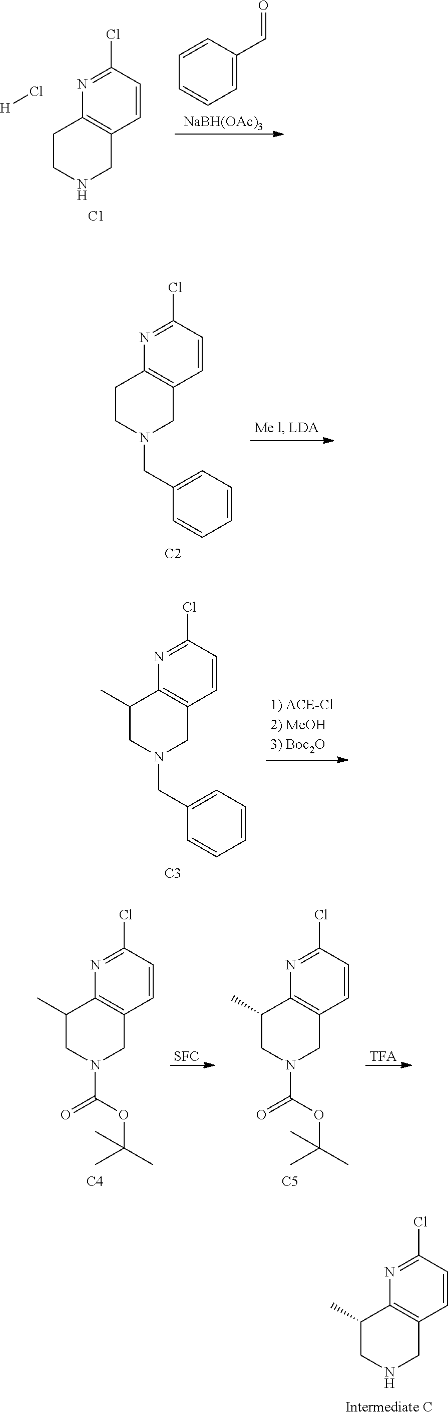

### Step 1: preparation of 6-benzyl-2-chloro-7,8-dihydro-5H-1,6-naphthyridine (compound C2)

2-Chloro-5,6,7,8-tetrahydro-1,6-naphthyridine hydrochloride (compound C1, CAS: 766545-20-4, Vendor: Bepharm, 40.0 g, 195.0 mmol) was suspended in DCM (500 mL). Then triethylamine (27.0 mL, 195.0 mmol) was added and the pH was adjusted to 5 with acetic acid (11.7 g, 195.0 mmol). Benzaldehyde (23.8 mL, 234.0 mmol) and sodium triacetoxyborohydride (62.0 g, 292.5 mmol) were added to the mixture which was stirred at 25° C. for 16 hours. The resulting solution was washed with 5% NaHCO₃aqueous solution (500 mL) and brine (300 mL), dried over with Na₂SO₄ and concentrated. The residue was purified by flash column eluting with a gradient of EA/PE (0% to 20%) to give compound C2 (40.0 g) as a light yellow solid. MS: calc'd 259 (MH⁺), measured 259 (MH⁺).

### Step 2: preparation of 6-benzyl-2-chloro-8-methyl-7,8-dihydro-5H-1,6-naphthyridine (compound C3)

To a solution of 6-benzyl-2-chloro-7,8-dihydro-5H-1,6-naphthyridine (compound C2, 35.0 g, 135.3 mmol) in THF (350 mL) was added lithium diisopropylamide (LDA) in THF (101.5 mL, 202.9 mmol) under the nitrogen at −70° C. and the mixture was stirred at −70° C. for 1 hour. Then iodomethane (11.0 mL, 175.9 mmol) was added dropwise at −70° C. to the mixture which was continued to be stirred at −70° C. for 3 hours. The reaction was quenched by adding saturated ammonium chloride solution (350 mL), extracted with EA (150 mL) three times. The combined organic layer was washed with brine, dried over Na₂SO₄ and concentrated. The residue was purified by flash column eluting with a gradient of EA/PE (10% to 30%) to give compound C3 (23.0 g) as a yellow oil. MS: calc'd 273 (MH⁺), measured 273 (MH⁺).

### Step 3: preparation of tert-butyl 2-chloro-8-methyl-7,8-dihydro-5H-1,6-naphthyridine-6-carboxylate (compound C4)

To a solution of 6-benzyl-2-chloro-8-methyl-7,8-dihydro-5H-1,6-naphthyridine (compound C3, 23.0 g, 84.3 mmol) in toluene (115 mL) was added 1-chloroethyl chloroformate (11.0 mL, 101.2 mmol). The mixture was stirred at 80° C. for 16 hours. The reaction mixture was concentrated to remove the solvent and the residue was dissolved in methanol (230 mL) and the mixture was stirred at 80° C. for 2 hours. After being cooled down to room temperature, the mixture was added into diisopropyl ether (30 mL). The resulting solid was collected by filtration and washed by diisopropyl ether.

A mixture of obtained solid and di-t-butyldicarbonate (23.9 g, 109.6 mmol) in THF (115 mL) and sodium hydroxide aqueous solution (1N, 114.7 mL, 114.7 mmol) was stirred at 25° C. for 16 hours. The reaction mixture was concentrated under reduced pressure to remove the THF, extracted with EA (100 mL) three times. The combined organic layer was washed with brine, dried over Na₂SO₄ and concentrated. The residue was purified by flash column eluting with a gradient of EA/PE (0% to 20%) to give compound C4 (20.7 g) as a white solid. MS: calc'd 283 (MH⁺), measured 283 (MH⁺). ¹H NMR (400 MHz, CHLOROFORM-d) 6=7.36 (d, J=8.19 Hz, 1H), 7.15 (d, J=8.07 Hz, 1H), 4.83-4.36 (m, 2H), 3.76-3.53 (m, 2H), 3.06 (br d, J=4.52 Hz, 1H), 1.50 (s, 9H), 1.33 (d, J=6.97 Hz, 3H).

### Step 4: preparation of tert-butyl (8S)-2-chloro-8-methyl-7,8-dihydro-5H-1,6-naphthyridine-6-carboxylate (compound C5)

Compound C5 (faster eluted) was separated from tert-butyl 2-chloro-8-methyl-7,8-dihydro-5H-1,6-naphthyridine-6-carboxylate (compound C4) by chiral SFC (Gradient: 10% ethanol (0.1% NH₃H₂O) in CO₂, Column: AD, 250×20 mm I.D., 5 μm). MS: calc'd 283 (MH⁺), measured 283 (MH⁺).

### Step 5: preparation of (8S)-2-chloro-8-methyl-5,6,7,8-tetrahydro-1,6-naphthyridine (Intermediate C)

To a solution of tert-butyl (8S)-2-chloro-8-methyl-7,8-dihydro-5H-1,6-naphthyridine-6-carboxylate (compound C5, 460 mg, 1.6 mmol) in DCM (2.5 mL) was added 2,2,2-trifluoroacetic acid (5 mL). The reaction mixture was stirred at room temperature for 1 hour. The reaction mixture was concentrated. The residue was used directly for next step reaction. MS: calc'd 183 (MH⁺), measured 183 (MH⁺).

### Intermediate D

### (8R)-2-chloro-8-methyl-5,6,7,8-tetrahydro-1,6-naphthyridine

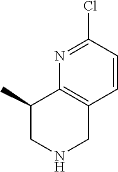

The title compound was prepared according to the following scheme:

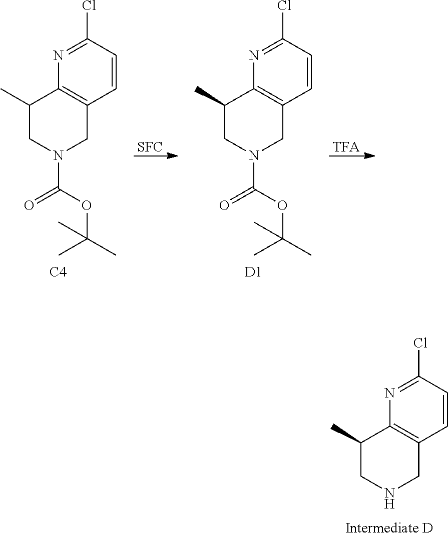

The title compound was prepared in analogy to the preparation of Intermediate C by using tert-butyl (8R)-2-chloro-8-methyl-7,8-dihydro-5H-1,6-naphthyridine-6-carboxylate (compound D1, the other isomer (slower eluted) separated from compound C4) instead of tert-butyl (8S)-2-chloro-8-methyl-7,8-dihydro-5H-1,6-naphthyridine-6-carboxylate (compound C5).

Intermediate D was obtained as a yellow oil and used directly for next step reaction. MS: calc'd 183 (MH⁺), measured 183 (MH⁺).

### Intermediate E

### (4R)-6-chloro-4-methyl-1,2,3,4-tetrahydro-2,7-naphthyridine

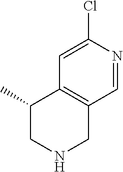

The title compound was prepared according to the following scheme:

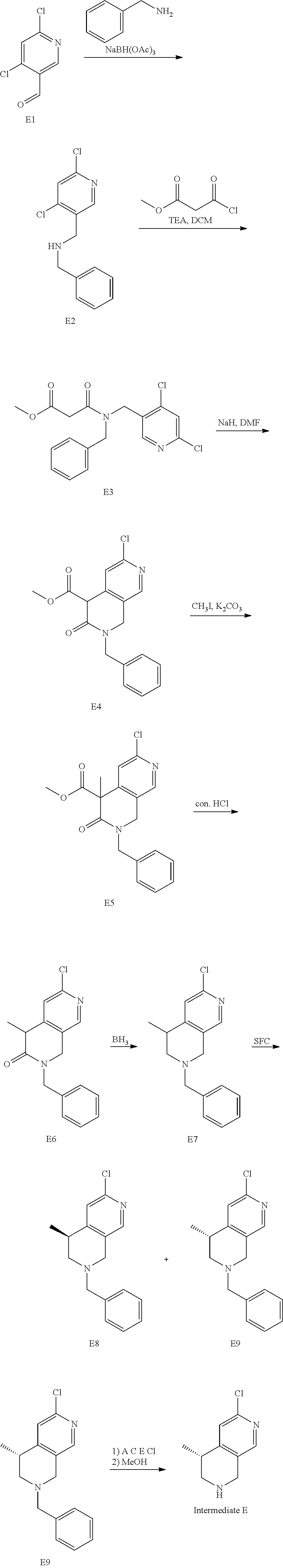

### Step 1: preparation of N-[(4,6-dichloro-3-pyridyl)methyl]-1-phenyl-methanamine (compound E2)

To the solution of 4,6-dichloropyridine-3-carbaldehyde (compound E1, CAS: 1060811-62-2, Vendor: PharmaBlock, 30.0 g, 170.5 mmol) in methanol (150 mL) under 0° C. was added benzylamine (28.0 mL, 255.7 mmol) and acetic acid (5.1 g, 85.2 mmol). The mixture was stirred at 20° C. for 3 hours under nitrogen atmosphere and then cooled to 0° C. again. Sodium cyanoborohydride (16.1 g, 255.7 mmol) was added to the solution. The reaction was warmed to 20° C. and stirred at 20° C. for another 15 hours under nitrogen. The reaction mixture was then quenched with saturated aq. NaHCO₃solution and concentrated. The mixture was extracted with EA (500 mL) three times. The combined organic layer was dried over Na₂SO₄, filtered and concentrated in vacuum to afford compound E2 (50.0 g) as light yellow oil which was used for next step without further purification. MS calc'd 267 (MH⁺); measured 267 (MH⁺).

### Step 2: preparation of methyl 3-[benzyl-[(4,6-dichloro-3-pyridyl)methyl]amino]-3-oxo-propanoate (compound E3)

A solution of N-[(4,6-dichloro-3-pyridyl)methyl]-1-phenyl-methanamine (compound E2, 30.0 g, 112.3 mmol) in DCM (450 mL) was cooled to 0° C. Triethylamine (100 mL, 561.5 mmol) was added to the solution, followed by adding dropwise of methyl malonyl chloride (15.6 mL, 146.0 mmol) in DCM (50 mL) solution at 0° C. The reaction was allowed to warm to room temperature and stirred at 25° C. for 16 hours under nitrogen atmosphere. The mixture was added to ice-water (2000 mL) and extracted with DCM (1000 mL) three times. The organic layers were washed with water, brine, dried over Na₂SO₄, filtered and concentrated. The residue was purified by flash column eluting with a gradient of EA/PE (0% to 25%) to afford compound E3 (134.8 g) as a light yellow solid. MS: calc'd 367 (MH⁺), measured 367 (MH⁺).

### Step 3: preparation of methyl 2-benzyl-6-chloro-3-oxo-1,4-dihydro-2,7-naphthyridine-4-carboxylate (compound E4)

To a stirred solution of methyl 3-[benzyl-[(4,6-dichloro-3-pyridyl)methyl]amino]-3-oxo-propanoate (compound E3, 28.0 g, 73.4 mmol) in DMF (800 mL) was added NaH (7.3 g, 183.6 mmol) (60% w/w in mineral oil) at 0° C. The resulting mixture was then warmed up to 120° C. and stirred at 120° C. for 2 hours under nitrogen atmosphere. The reaction mixture was cooled to room temperature, then poured into saturated aqueous NH₄Cl (800 mL) at ice-water bath. The mixture was extracted with EA (1500 mL) three times. The combined organic layer was washed with brine, dried over MgSO₄, filtered and concentrated to afford compound E4 (20.0 g) as a brown solid. MS: calc'd 331 (MH⁺), measured 331 (MH⁺).

### Step 4: preparation of methyl 2-benzyl-6-chloro-4-methyl-3-oxo-1H-2,7-naphthyridine-4-carboxylate (compound E5)

To a solution of methyl 2-benzyl-6-chloro-3-oxo-1,4-dihydro-2,7-naphthyridine-4-carboxylate (compound E4, 55.0 g, 166.3 mmol) in ACN (825 mL) was added K₂CO₃ (57.5 g, 415.7 mmol). The mixture was stirred at 20° C. for 1 hour. Then Mel (14.0 mL, 216.2 mmol) was added to the mixture and the reaction was stirred at 70° C. for 15 hours. After being cooled down to room temperature, the reaction was quenched by adding NH₃·H₂O (300 mL) and the reaction mixture was concentrated under reduced pressure to remove the solvent, extracted with EA (500 mL) three times. The organic layer was dried over Na₂SO₄, filtered and concentrated. The residue was purified by flash column eluting with a gradient of EA/PE (20% to 35%) to give compound E5 (25.0 g) as a light brown solid. MS: calc'd 345 (MH⁺), measured 345 (MH⁺).

### Step 5: preparation of 2-benzyl-6-chloro-4-methyl-1,4-dihydro-2,7-naphthyridin-3-one (Compound E6)

The mixture of methyl 2-benzyl-6-chloro-4-methyl-3-oxo-1H-2,7-naphthyridine-4-carboxylate (compound E5, 10.0 g, 29.0 mmol) in con. HCl (100.0 mL) was heated at 100° C. for 15 min. The reaction mixture was cooled down and the pH was adjusted to 8-9 with sat. NaHCO₃ at 5° C. The mixture was extracted with EA (800 mL) three times. The organic layer was washed with water, brine, dried over Na₂SO₄, filtered and concentrated. The residue was purified by flash column eluting with a gradient of EA/PE (5% to 50%) to give compound E6 (22.0 g) as brown oil. MS: calc'd 287 (MH⁺), measured 287 (MH⁺).

### Step 6: preparation of 2-benzyl-6-chloro-4-methyl-3,4-dihydro-1H-2,7-naphthyridine (compound E7)

2-benzyl-6-chloro-4-methyl-1,4-dihydro-2,7-naphthyridin-3-one (Compound E6, 5.0 g, 17.4 mmol) was charged in three-neck flask and purged with nitrogen gas. BH₃·THF (150.0 mL, 150 mmol) (1M in THF) was added dropwise to the flask at 0° C. via cannula. The reaction was stirred at 20° C. for 30 minutes and then heated at 70° C. for 15 hours. After being cooled to room temperature, methanol (100 mL) was added dropwise to the reaction solution. The mixture was stirred at 20° C. for 30 minutes, then 1 N HCl (50 mL) was added dropwise. The mixture was stirred at 20° C. for 30 minutes and heated at 70° C. for 2 hours, then concentrated to remove organic solvent. The residue was cooled to 0° C. and poured into sat. NaHCO₃ aq. solution at ice-water bath. The mixture was extracted with EA (50 mL) three times. The organic layer was dried over Na₂SO₄, filtered and concentrated. The residue was purified by flash column to afford compound E7 (2.0 g) as a light yellow solid. MS: calc'd 273 (MH⁺), measured 273 (MH⁺).

### Step 7: preparation of (4S)-2-benzyl-6-chloro-4-methyl-3,4-dihydro-1H-2,7-naphthyridine (compound E8) & (4R)-2-benzyl-6-chloro-4-methyl-3,4-dihydro-1H-2,7-naphthyridine (compound E9)

The title compounds were separated from 2-benzyl-6-chloro-4-methyl-3,4-dihydro-1H-2,7-naphthyridine (compound E7, 6.4 g) by chiral SFC (Gradient: 5%-40 methanol (0.05% DEA) in CO₂, Column: Chiralpak AD-3, 50×4.6 mm I.D., 3 μm). (4S)-2-benzyl-6-chloro-4-methyl-3,4-dihydro-1H-2,7-naphthyridine (compound E8, faster eluted, 2.8 g) and (4R)-2-benzyl-6-chloro-4-methyl-3,4-dihydro-1H-2,7-naphthyridine (compound E9, slower eluted, 2.8 g) were obtained as light yellow solid. MS: calc'd 273 (MH⁺), measured 273 (MH⁺).

### Step 8: preparation of (4R)-6-chloro-4-methyl-1,2,3,4-tetrahydro-2,7-naphthyridine (Intermediate E)

To a solution of (4R)-2-benzyl-6-chloro-4-methyl-3,4-dihydro-1H-2,7-naphthyridine (compound E9, 2.8 g, 10.3 mmol) and DIEA (5.4 mL, 30.9 mmol) in DCE (60 mL) at 0° C. was added dropwise of 1-chloroethyl chloroformate (4.4 mL, 40.7 mmol). The reaction mixture was stirred at 0° C. for 15 minutes, warmed to 25° C. in 1 hour, then heated at 70° C. for 2 hours. The solvent was removed and methanol (30 mL) was added. The reaction mixture was heated at 70° C. for 2 hours, then concentrated, the residue was dissolved in EA (50 mL) and sat. NaHCO₃ aq. solution (60 mL). The mixture was extracted with EA (40 mL) three times. The combined organic layer was washed with brine (50 mL), dried over Na₂SO₄ and concentrated. The residue was purified by flash column eluting with a gradient of EA/PE (0% to 100%) and MeOH/DCM (10%) to afford Intermediate E (1.2 g) as a light brown oil. MS: calc'd 183 (MH⁺), measured 183 (MH⁺).

### Example 1

### (4R)-4-methyl-2-(1-methylpyrazolo[3,4-b]pyridin-4-yl)-6-piperazin-1-yl-3,4-dihydro-1H-isoquinoline

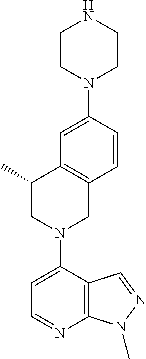 [SMILES: C[C@H]1CN(c2ccnc3c2cnn3C)Cc2ccc(N3CCNCC3)cc21]

The title compound was prepared according to the following scheme:

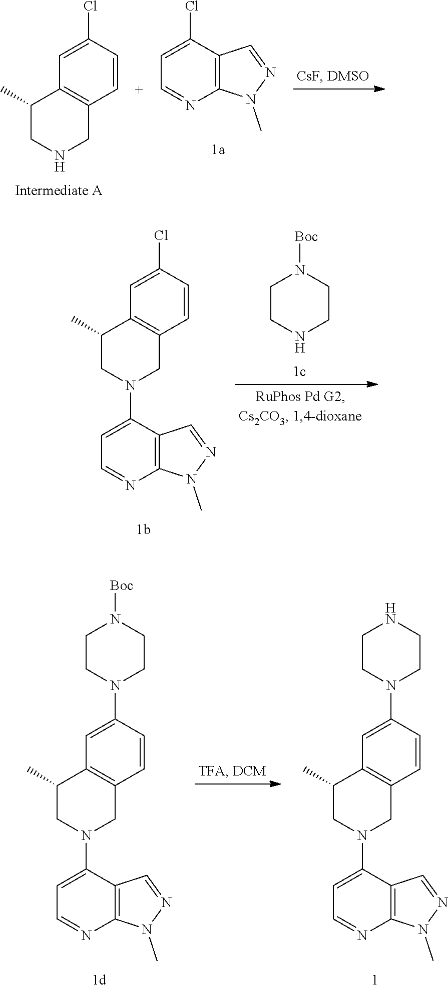

### Step 1: preparation of (4R)-6-chloro-4-methyl-2-(1-methylpyrazolo[3,4-b]pyridin-4-yl)-3,4-dihydro-1H-isoquinoline (compound 1b)

To a solution of 4-chloro-1-methyl-pyrazolo[3,4-b]pyridine (compound 1a, CAS: 1268520-92-8. Vendor: PharmaBlock. 77 mg, 461 μmol) in DMSO (2 mL) was added (4R)-6-chloro-4-methyl-1,2,3,4-tetrahydroisoquinoline (Intermediate A, 50 mg, 231 μmol) and CsF (175 mg, 1.15 mmol). The reaction mixture was stirred at 130° C. overnight. After being cooled to room temperature, the reaction mixture was diluted with EA (50 mL), washed with water (30 mL) for four times, dried over Na₂SO₄, filtered and concentrated in vacuo. The residue was purified by flash column eluting with a gradient of EA(with 10% MeOH)/PE (0% to 50%) to afford compound 1b. MS: calc'd 313 (MH⁺), measured 313 (MH⁺).

### Step 2: preparation of tert-butyl 4-[(4R)-4-methyl-2-(1-methylpyrazolo[3,4-b]pyridin-4-yl)-3,4-dihydro-1H-isoquinolin-6-yl]piperazine-1-carboxylate (compound 1d)

The mixture of (4R)-6-chloro-4-methyl-2-(1-methylpyrazolo[3,4-b]pyridin-4-yl)-3,4-dihydro-1H-isoquinoline (compound 1b, 28 mg, 90 μmol), tert-butyl piperazine-1-carboxylate (CAS: 57260-71-6, Vendor: Accela, compound 1c, 33 mg, 179 μmol), RuPhos Pd G2 (14 mg, 18 μmol) and Cs₂CO₃ (88 mg, 269 μmol) in 1,4-dioxane (5 mL) was charged with N2, and the mixture was heated to 110° C. overnight. After being cooled to room temperature, the solid was filtered off and washed with EA (10 mL) for two times. The combined mixture was concentrated and purified by flash column eluting with a gradient of EA/PE (0% to 100%) to afford compound 1d. MS: calc'd 463 (MH⁺), measured 463 (MH⁺).

### Step 3: preparation of (4R)-4-methyl-2-(1-methylpyrazolo[3,4-b]pyridin-4-yl)-6-piperazin-1-yl-3,4-dihydro-1H-isoquinoline (Example 1)

The compound 1d was dissolved in DCM (5 mL) and TFA (1 mL) and stirred at rt for 4 h. The mixture was concentrated and purified by prep-HPLC to give Example 1 (25 mg) as a yellow solid. MS: calc'd 363 (MH⁺), measured 363 (MH⁺). ¹H NMR (400 MHz, METHANOL-d₄) δ=8.56 (s, 1H), 8.08 (d, J=7.5 Hz, 1H), 7.31 (d, J=8.4 Hz, 1H), 7.01 (d, J=2.2 Hz, 1H), 6.98 (d, J=8.6 Hz, 1H), 6.86 (d, J=7.5 Hz, 1H), 5.19-5.04 (m, 1H), 5.00-4.95 (m, 1H), 4.09 (s, 3H), 4.04 (br dd, J=4.6, 16.3 Hz, 1H), 3.89 (br dd, J=6.7, 12.2 Hz, 1H), 3.49-3.36 (m, 8H), 3.34-3.28 (m, 1H), 1.43 (d, J=7.0 Hz, 3H). The configuration of the stereo center was confirmed by co-crystal structure with protein TLR8.

### Example 2

### (4R)-2-(1,6-dimethylpyrazolo[3,4-b]pyridin-4-yl)-4-methyl-6-piperazin-1-yl-3,4-dihydro-1H-isoquinoline

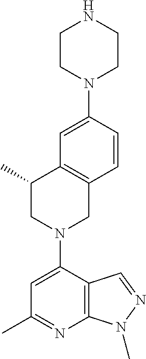 [SMILES: Cc1cc(N2Cc3ccc(N4CCNCC4)cc3[C@@H](C)C2)c2cnn(C)c2n1]

The title compound was prepared in analogy to the preparation of Example 1 by using 4-chloro-1,6-dimethyl-pyrazolo[3,4-b]pyridine (CAS: 19867-78-8, Vendor: PharmaBlock) instead of compound 1a. Example 2 (15 mg) was obtained as a yellow solid. MS: calc'd 377 (MH⁺), measured 377 (MH⁺). ¹H NMR (400 MHz, METHANOL-d₄) δ=8.48 (br s, 1H), 7.30 (d, J=8.2 Hz, 1H), 7.02-6.96 (m, 2H), 6.71 (s, 1H), 5.19-5.01 (m, 1H), 5.01-4.91 (m, 1H), 4.08 (s, 3H), 4.07-3.97 (m, 1H), 3.93-3.81 (m, 1H), 3.47-3.42 (m, 4H), 3.42-3.36 (m, 4H), 3.29-3.24 (m, 1H), 2.66 (s, 3H), 1.42 (d, J=7.0 Hz, 3H).

### Example 3

### 2-(1-methylpyrazolo[3,4-b]pyridin-4-yl)-6-piperazin-1-yl-3,4-dihydro-1H-isoquinoline

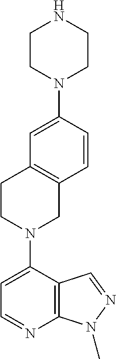 [SMILES: Cn1ncc2c(N3CCc4cc(N5CCNCC5)ccc4C3)ccnc21]

The title compound was prepared in analogy to the preparation of Example 1 by using 6-bromo-1,2,3,4-tetrahydroisoquinoline (CAS: 226942-29-6, Vendor: Shuya) instead of Intermediate A, and replacing CsF with DIPEA in the SNAr reaction. Example 3 (35 mg) was obtained as a yellow solid. MS: calc'd 349 (MH⁺), measured 349 (MH⁺). ¹H NMR (400 MHz, METHANOL-d₄) δ=8.51 (s, 1H), 8.07 (d, J=7.2 Hz, 1H), 7.30 (d, J=8.3 Hz, 1H), 7.02-6.95 (m, 2H), 6.81 (d, J=7.3 Hz, 1H), 5.01-4.94 (m, 2H), 4.08 (s, 3H), 4.07-4.03 (m, 2H), 3.46-3.41 (m, 4H), 3.40-3.36 (m, 4H), 3.33 (br s, 1H), 3.17-3.11 (m, 2H).

### Example 4

### 3-[4-methyl-2-(1-methylpyrazolo[3,4-b]pyridin-4-yl)-3,4-dihydro-1H-isoquinolin-6-yl]-3,9-diazaspiro[5.5]undecane

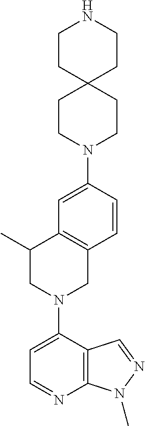 [SMILES: CC1CN(c2ccnc3c2cnn3C)Cc2ccc(N3CCC4(CCNCC4)CC3)cc21]

The title compound was prepared in analogy to the preparation of Example 1 by using 6-bromo-4-methyl-1,2,3,4-tetrahydroisoquinoline hydrochloride (CAS: 2227205-04-9, Vendor: PharmaBlock) and tert-butyl 3,9-diazaspiro[5.5]undecane-3-carboxylate (CAS: 173405-78-2, Vendor: PharmaBlock) instead of Intermediate A and 1c. Example 4 (12 mg) was obtained as a yellow gum. MS: calc'd 431 (MH⁺), measured 431 (MH⁺). ¹H NMR (400 MHz, METHANOL-d₄) δ=8.58 (s, 1H), 8.09 (d, J=7.5 Hz, 1H), 7.48-7.41 (m, 1H), 7.40-7.26 (m, 2H), 6.88 (d, J=7.5 Hz, 1H), 5.25-5.14 (m, 1H), 5.07-4.97 (m, 1H), 4.09 (s, 3H), 4.07 (br d, J=6.7 Hz, 1H), 3.90 (dd, J=7.0, 12.2 Hz, 1H), 3.55-3.42 (m, 4H), 3.42-3.34 (m, 1H), 3.27-3.21 (m, 4H), 1.97-1.80 (m, 8H), 1.45 (d, J=7.0 Hz, 3H).

### Example 5

### 3-[2-(1,6-dimethylpyrazolo[3,4-b]pyridin-4-yl)-4-methyl-3,4-dihydro-1H-isoquinolin-6-yl]-3,9-diazaspiro[5.5]undecane

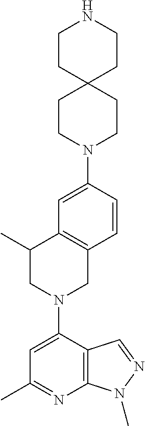 [SMILES: Cc1cc(N2Cc3ccc(N4CCC5(CCNCC5)CC4)cc3C(C)C2)c2cnn(C)c2n1]

The title compound was prepared in analogy to the preparation of Example 1 by using 6-chloro-4-methyl-1,2,3,4-tetrahydroisoquinoline (CAS: 73037-81-7, Vendor: Bepharm) instead of Intermediate A, 4-chloro-1,6-dimethyl-pyrazolo[3,4-b]pyridine (CAS: 19867-78-8, Vendor: PharmaBlock) instead of compound 1a and tert-butyl 3,9-diazaspiro[5.5]undecane-3-carboxylate (CAS: 173405-78-2, Vendor: PharmaBlock) instead of compound 1c. Example 5 (20 mg) was obtained as a yellow solid. MS: calc'd 445 (MH⁺), measured 445 (MH⁺). ¹H NMR (400 MHz, METHANOL-d₄) δ=8.50 (s, 1H), 7.52-7.46 (m, 1H), 7.46-7.43 (m, 1H), 7.41-7.35 (m, 1H), 6.74 (s, 1H), 5.24-5.12 (m, 1H), 5.07-4.96 (m, 1H), 4.13-4.01 (m, 4H), 3.93-3.82 (m, 1H), 3.59-3.50 (m, 4H), 3.39-3.33 (m, 1H), 3.27-3.19 (m, 4H), 2.67 (s, 3H), 2.00-1.93 (m, 4H), 1.92-1.81 (m, 4H), 1.45 (d, J=7.0 Hz, 3H).

### Example 6

### 3-[4,4-dimethyl-2-(1-methylpyrazolo[3,4-b]pyridin-4-yl)-1,3-dihydroisoquinolin-6-yl]-3,9-diazaspiro[5.5]undecane

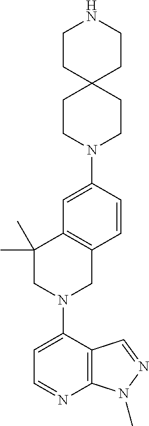 [SMILES: Cn1ncc2c(N3Cc4ccc(N5CCC6(CCNCC6)CC5)cc4C(C)(C)C3)ccnc21]

The title compound was prepared in analogy to the preparation of Example 1 by using 6-bromo-4,4-dimethyl-2,3-dihydro-1H-isoquinoline hydrochloride (CAS: 1203684-61-0, Vendor: Bepharm) instead of Intermediate A and tert-butyl 3,9-diazaspiro[5.5]undecane-3-carboxylate (CAS: 173405-78-2, Vendor: PharmaBlock) instead of compound 1c. Example 6 (19 mg) was obtained as a yellow solid. MS: calc'd 445 (MH⁺), measured 445 (MH⁺). ¹H NMR (400 MHz, METHANOL-d₄) δ=8.66 (s, 1H), 8.14 (d, J=7.5 Hz, 1H), 7.71 (d, J=2.0 Hz, 1H), 7.61-7.54 (m, 2H), 6.98 (d, J=7.5 Hz, 1H), 5.28 (s, 2H), 4.13 (s, 3H), 3.97 (s, 2H), 3.71-3.59 (m, 4H), 3.31-3.23 (m, 4H), 2.05 (br t, J=5.6 Hz, 4H), 1.96-1.89 (m, 4H), 1.48 (s, 6H).

### Example 7

### 4-piperidyl-[4-[(4R)-4-methyl-2-(1-methylpyrazolo[3,4-b]pyridin-4-yl)-3,4-dihydro-1H-isoquinolin-6-yl]piperazin-1-yl]methanone

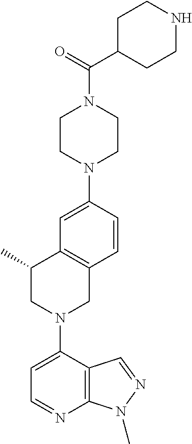 [SMILES: C[C@H]1CN(c2ccnc3c2cnn3C)Cc2ccc(N3CCN(C(=O)C4CCNCC4)CC3)cc21]

The title compound was prepared according to the following scheme:

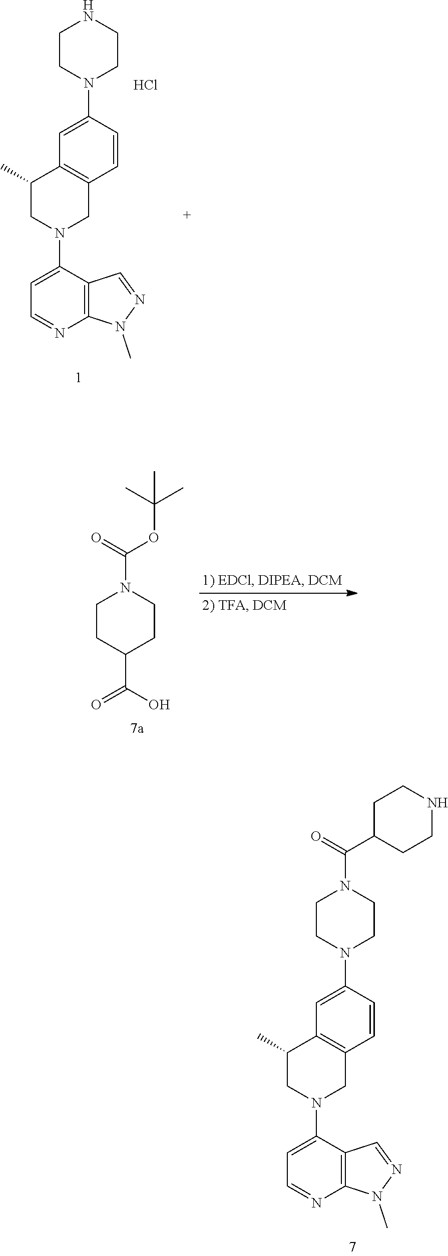 [SMILES: C[C@H]1CN(c2ccnc3c2cnn3C)Cc2ccc(N3CCNCC3)cc21 ; Cl ; CC(C)(C)OC(=O)N1CCC(C(=O)O)CC1 ; C[C@H]1CN(c2ccnc3c2cnn3C)Cc2ccc(N3CCN(C(=O)C4CCNCC4)CC3)cc21]

### Step 1: preparation of 4-piperidyl-[4-[(4R)-4-methyl-2-(1-methylpyrazolo[3,4-b]pyridin-4-yl)-3,4-dihydro-1H-isoquinolin-6-yl]piperazin-1-yl]methanone (Example 7)

To a flask was added (4R)-4-methyl-2-(1-methylpyrazolo[3,4-b]pyridin-4-yl)-6-piperazin-1-yl-3,4-dihydro-1H-isoquinoline hydrochloride (HCl salt of Example 1, 50 mg, 125 μmol), 1-tert-butoxycarbonylpiperidine-4-carboxylic acid (compound 7a, CAS: 84358-13-4, Vendor: Accela, 43 mg, 188 μmol), EDCI (36 mg, 188 μmol), DIPEA (88 μL, 501 μmol) and DCM (5 mL). The mixture was stirred at rt overnight. After concentration, the mixture was purified by flash column eluting with a gradient of EA(with 10% MeOH)/PE (0% to 70%) to afford a yellow oil which was re-dissolved in DCM (5 mL) and TFA (2 mL) and stirred at rt for 2 h. After concentration, the mixture was purified by reversed flash column eluting with a gradient of ACN/Water (with 0.05% TFA) (0 to 30%) to give Example 7 (30 mg) as a yellow solid. MS: calc'd 474 (MH⁺), measured 474 (MH⁺). ¹H NMR (400 MHz, METHANOL-d₄) δ=8.55 (s, 1H), 8.07 (d, J=7.3 Hz, 1H), 7.27 (d, J=8.2 Hz, 1H), 6.99-6.93 (m, 2H), 6.85 (d, J=7.3 Hz, 1H), 5.11 (br dd, J=1.3, 16.7 Hz, 1H), 4.98-4.90 (m, 1H), 4.08 (s, 3H), 4.07-4.01 (m, 1H), 3.88 (br dd, J=7.2, 12.7 Hz, 1H), 3.81-3.73 (m, 4H), 3.49-3.42 (m, 2H), 3.26-3.17 (m, 4H), 3.17-3.05 (m, 4H), 2.02-1.89 (m, 4H), 1.42 (d, J=7.0 Hz, 3H).

### Example 8

### pyrrolidin-2-yl-[4-[(4R)-4-methyl-2-(1-methylpyrazolo[3,4-b]pyridin-4-yl)-3,4-dihydro-1H-isoquinolin-6-yl]piperazin-1-yl]methanone

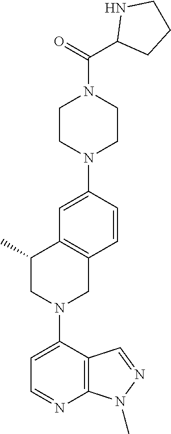 [SMILES: C[C@H]1CN(c2ccnc3c2cnn3C)Cc2ccc(N3CCN(C(=O)C4CCCN4)CC3)cc21]

The title compound was prepared in analogy to the preparation of Example 7 by using 1-tert-butoxycarbonylpyrrolidine-2-carboxylic acid (CAS: 59433-50-0, Vendor: Accela) instead of 1-tert-butoxycarbonylpiperidine-4-carboxylic acid (compound 7a). Example 8 (30 mg) was obtained as a yellow solid. MS: calc'd 460 (MH⁺), measured 460 (MH⁺). ¹H NMR (400 MHz, METHANOL-d₄) δ=8.55 (s, 1H), 8.07 (d, J=7.3 Hz, 1H), 7.28 (d, J=8.6 Hz, 1H), 7.00-6.94 (m, 2H), 6.84 (d, J=7.5 Hz, 1H), 5.11 (br d, J=15.8 Hz, 1H), 4.97-4.92 (m, 1H), 4.70 (dd, J=7.5, 8.6 Hz, 1H), 4.08 (s, 3H), 4.05-4.02 (m, 1H), 3.88 (br dd, J=6.9, 12.5 Hz, 1H), 3.81 (td, J=5.2, 8.0 Hz, 2H), 3.75-3.65 (m, 2H), 3.50-3.40 (m, 1H), 3.40-3.33 (m, 2H), 3.28-3.22 (m, 4H), 2.58-2.50 (m, 1H), 2.15-2.04 (m, 2H), 2.04-1.95 (m, 1H), 1.42 (d, J=7.0 Hz, 3H).

### Example 9

### 7-[4-[(4R)-4-methyl-2-(1-methylpyrazolo[3,4-b]pyridin-4-yl)-3,4-dihydro-1H-isoquinolin-6-yl]piperazin-1-yl]-1,2,3,4-tetrahydro-2,6-naphthyridine

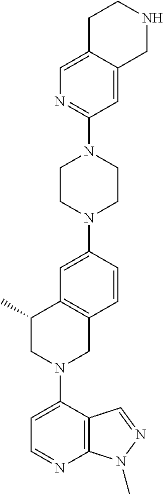 [SMILES: C[C@H]1CN(c2ccnc3c2cnn3C)Cc2ccc(N3CCN(c4cc5c(cn4)CCNC5)CC3)cc21]

The title compound was prepared according to the following scheme:

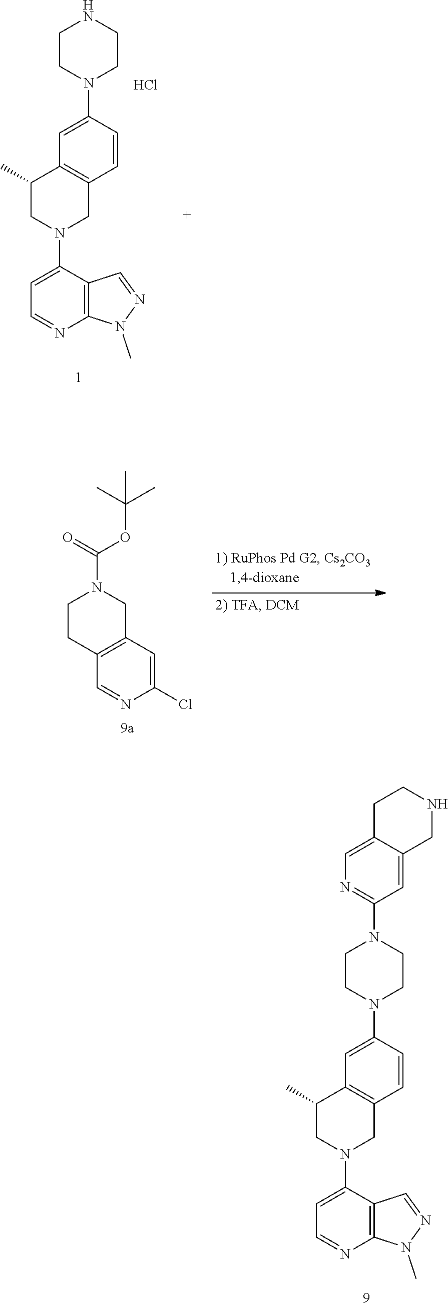 [SMILES: C[C@H]1CN(c2ccnc3c2cnn3C)Cc2ccc(N3CCNCC3)cc21 ; Cl ; CC(C)(C)OC(=O)N1CCc2cnc(Cl)cc2C1 ; C[C@H]1CN(c2ccnc3c2cnn3C)Cc2ccc(N3CCN(c4cc5c(cn4)CCNC5)CC3)cc21]

### Step 1: preparation of 7-[4-[(4R)-4-methyl-2-(1-methylpyrazolo[3,4-b]pyridin-4-yl)-3,4-dihydro-1H-isoquinolin-6-yl]piperazin-1-yl]-1,2,3,4-tetrahydro-2,6-naphthyridine

### Example 9

The mixture of (4R)-4-methyl-2-(1-methylpyrazolo[3,4-b]pyridin-4-yl)-6-piperazin-1-yl-3,4-dihydro-1H-isoquinoline hydrochloride (HCl salt of Example 1, 50 mg, 125 μmol), tert-butyl 7-chloro-3,4-dihydro-1H-2,6-naphthyridine-2-carboxylate (51 mg, 188 μmol), RuPhos Pd G2 (10 mg, 13 μmol) and Cs₂CO₃ (163 mg, 501 μmol) in 1,4-dioxane (5 mL) was charged with N₂, and the mixture was heated to 110° C. overnight. After being cooled to room temperature, the solid was filtered off and washed with EA (10 mL) for two times. The combined organic solvent was concentrated and purified by flash column eluting with a gradient of EA(with 10% MeOH)/PE (0% to 70%) to afford a yellow oil which was re-dissolved in DCM (5 mL) and TFA (2 mL) and stirred at rt for 2 h. After concentration, the mixture was purified by reversed flash column eluting with a gradient of ACN/Water (with 0.05% TFA) (0 to 30%) to give Example 9 (51 mg) as a yellow solid. MS: calc'd 495 (MH⁺), measured 495 (MH⁺). ¹H NMR (400 MHz, METHANOL-d₄) δ=8.56 (s, 1H), 8.08 (d, J=7.5 Hz, 1H), 8.03 (s, 1H), 7.30 (d, J=8.3 Hz, 1H), 7.17 (br s, 1H), 7.04-6.97 (m, 2H), 6.86 (d, J=7.5 Hz, 1H), 5.20-5.05 (m, 1H), 5.01-4.94 (m, 1H), 4.46 (s, 2H), 4.09 (s, 3H), 4.07-3.98 (m, 1H), 3.89 (br dd, J=6.7, 12.0 Hz, 1H), 3.85-3.73 (m, 4H), 3.53 (t, J=6.3 Hz, 2H), 3.45-3.39 (m, 4H), 3.37-3.33 (m, 1H), 3.07 (t, J=6.2 Hz, 2H), 1.44 (d, J=7.0 Hz, 3H).

### Example 10

### (4R)-4-methyl-2-(1-methylpyrazolo[3,4-b]pyridin-4-yl)-6-(4-piperazin-1-yl-1-piperidyl)-3,4-dihydro-1H-isoquinoline

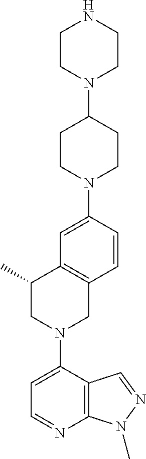 [SMILES: C[C@H]1CN(c2ccnc3c2cnn3C)Cc2ccc(N3CCC(N4CCNCC4)CC3)cc21]

The title compound was prepared in analogy to the preparation of Example 1 by using tert-butyl 4-(4-piperidyl)piperazine-1-carboxylate (CAS: 205059-24-1, Vendor: Bepharm) instead of compound 1c. Example 10 (5 mg) was obtained as a light yellow solid. MS: calc'd 446 (MH⁺), measured 446 (MH⁺). ¹H NMR (400 MHz, METHANOL-d₄) δ=8.27 (s, 1H), 8.09 (d, J=5.9 Hz, 1H), 7.17 (d, J=8.2 Hz, 1H), 6.91 (s, 2H), 6.45 (d, J=6.0 Hz, 1H), 4.82 (br s, 1H), 4.73-4.61 (m, 1H), 3.99 (s, 3H), 3.87-3.72 (m, 4H), 3.22-3.13 (m, 1H), 2.93 (br t, J=4.9 Hz, 4H), 2.76-2.60 (m, 6H), 2.46-2.34 (m, 1H), 2.00 (br d, J=12.3 Hz, 2H), 1.73-1.60 (m, 2H), 1.38 (d, J=7.0 Hz, 3H).

### Example 11

### (3R,4R)-4-methoxy-1-[1-[(4R)-4-methyl-2-(1-methylpyrazolo[3,4-b]pyridin-4-yl)-3,4-dihydro-1H-isoquinolin-6-yl]-4-piperidyl]pyrrolidin-3-amine

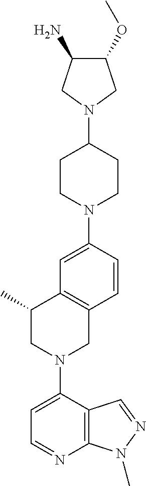 [SMILES: CO[C@@H]1CN(C2CCN(c3ccc4c(c3)[C@@H](C)CN(c3ccnc5c3cnn5C)C4)CC2)C[C@H]1N]

The title compound was prepared according to the following scheme:

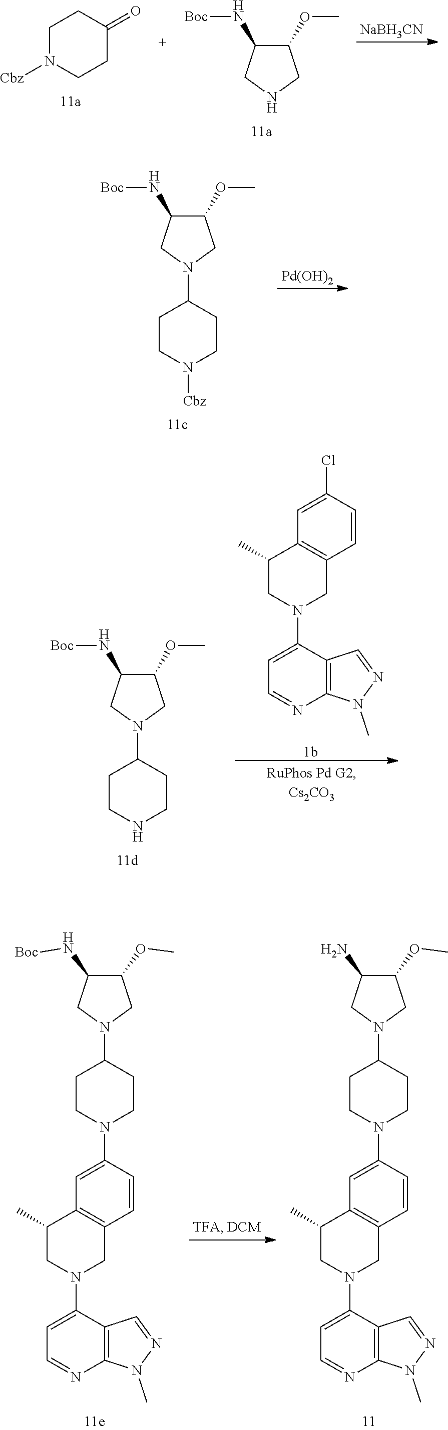

### Step 1: preparation of benzyl 4-[(3R,4R)-3-(tert-butoxycarbonylamino)-4-methoxy-pyrrolidin-1-yl]piperidine-1-carboxylate (compound 11c)

To a solution of benzyl 4-oxopiperidine-1-carboxylate (compound 11a, CAS: 19099-93-5, Vendor: Accela, 270 mg, 1.16 mmol) in ethanol (15 mL) was added tert-butyl N-[(3R,4R)-4-methoxypyrrolidin-3-yl]carbamate (compound 11b, CAS: 1932066-52-8, Vendor: PharmaBlock, 250 mg, 1.16 mmol) and acetic acid (33 μL, 557 μmol). The mixture was stirred at 50° C. for 1 h. Then NaBH₃CN (CAS: 25895-60-7, Vendor: Accela, 145 mg, 2.31 mmol) was added. The mixture was stirred at 50° C. overnight. After being cooled to room temperature, the reaction was quenched by adding saturated NaHCO₃ (10 mL) aqueous solution. The mixture was extracted by EA (10 mL) three times and the organic phase was washed with brine (20 mL) twice, dried over Na₂SO₄, filtered and concentrated. The residue was purified by flash column to afford compound 11c (160 mg). MS: calc'd 434 (MH⁺), measured 434 (MH⁺).

### Step 2: preparation of tert-butyl N-[(3R,4R)-4-methoxy-1-(4-piperidyl)pyrrolidin-3-yl]carbamate (compound 11d)

To a solution of benzyl 4-[(3R,4R)-3-(tert-butoxycarbonylamino)-4-methoxy-pyrrolidin-1-yl]piperidine-1-carboxylate (compound 11c, 160 mg, 369 μmol) in MeOH (15 mL) was added palladium hydroxide (20% on carbon, wetted with ca. 50% Water) (CAS: 12135-22-7, Vendor: TCI, 52 mg, 369 μmol). The mixture was flushed with hydrogen and stirred at rt for 2 h. The reaction mixture was filtrated and concentrated to give crude compound 11d (110 mg) which was used in next step without further purification. MS: calc'd 300 (MH⁺), measured 300 (MH⁺).

### Step 3: preparation of (3R,4R)-4-methoxy-1-[1-[(4R)-4-methyl-2-(1-methylpyrazolo[3,4-b]pyridin-4-yl)-3,4-dihydro-1H-isoquinolin-6-yl]-4-piperidyl]pyrrolidin-3-amine (Example 11)

The title compound was prepared in analogy to the preparation of Example 1 by using tert-butyl N-[(3R,4R)-4-methoxy-1-(4-piperidyl)pyrrolidin-3-yl]carbamate (compound 11d) instead of compound 1c. Example 11 (10 mg) was obtained as a white solid. MS: calc'd 476 (MH⁺), measured 476 (MH⁺). ¹H NMR (400 MHz, METHANOL-d₄) δ=8.26 (s, 1H), 8.09 (d, J=5.9 Hz, 1H), 7.16 (d, J=8.2 Hz, 1H), 6.94-6.84 (m, 2H), 6.44 (d, J=6.0 Hz, 1H), 4.81 (s, 1H), 4.73-4.62 (m, 1H), 3.99 (s, 3H), 3.87-3.64 (m, 4H), 3.58 (td, J=3.2, 6.4 Hz, 1H), 3.35 (s, 3H), 3.29-3.24 (m, 1H), 3.21-3.13 (m, 1H), 3.07 (dd, J=7.0, 9.4 Hz, 1H), 2.93 (dd, J=6.5, 10.5 Hz, 1H), 2.80-2.67 (m, 3H), 2.33 (dd, J=6.1, 9.5 Hz, 1H), 2.28-2.17 (m, 1H), 2.00 (br d, J=11.9 Hz, 2H), 1.62 (dt, J=3.2, 11.7 Hz, 2H), 1.38 (d, J=6.8 Hz, 3H).

### Example 12

### morpholin-2-yl-[4-[(4R)-4-methyl-2-(1-methylpyrazolo[3,4-b]pyridin-4-yl)-3,4-dihydro-1H-isoquinolin-6-yl]piperazin-1-yl]methanone

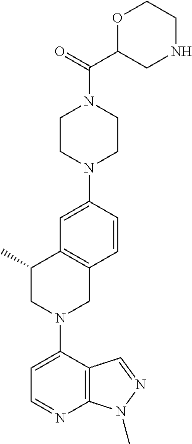 [SMILES: C[C@H]1CN(c2ccnc3c2cnn3C)Cc2ccc(N3CCN(C(=O)C4CNCCO4)CC3)cc21]

The title compound was prepared in analogy to the preparation of Example 7 by using 4-tert-butoxycarbonylmorpholine-2-carboxylic acid (CAS: 189321-66-2, Vendor: Accela) instead of 1-tert-butoxycarbonylpiperidine-4-carboxylic acid (compound 7a). Example 12 (26 mg) was obtained as a yellow solid. MS: calc'd 476 (MH⁺), measured 476 (MH⁺). ¹H NMR (400 MHz, METHANOL-d₄) δ=8.55 (s, 1H), 8.07 (d, J=7.3 Hz, 1H), 7.27 (d, J=8.3 Hz, 1H), 7.00-6.91 (m, 2H), 6.84 (d, J=7.5 Hz, 1H), 5.17-5.03 (m, 1H), 4.96-4.91 (m, 1H), 4.80-4.76 (m, 1H), 4.08 (s, 3H), 4.07-3.82 (m, 7H), 3.77-3.60 (m, 2H), 3.49-3.42 (m, 1H), 3.41-3.35 (m, 2H), 3.35-3.32 (m, 2H), 3.26-3.10 (m, 3H), 1.42 (d, J=7.0 Hz, 3H).

### Example 13

### 4-[(dimethylamino)methyl]-1-[(4R)-4-methyl-2-(1-methylpyrazolo[3,4-b]pyridin-4-yl)-3,4-dihydro-1H-isoquinolin-6-yl]piperidin-4-ol

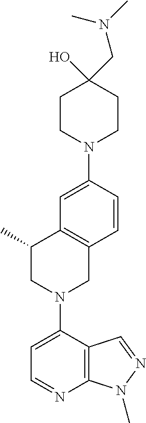 [SMILES: C[C@H]1CN(c2ccnc3c2cnn3C)Cc2ccc(N3CCC(O)(CN(C)C)CC3)cc21]

The title compound was prepared in analogy to the preparation of compound 1d by using 4-[(dimethylamino)methyl]piperidin-4-ol dihydrochloride (CAS: 695145-47-2, Vendor: J&K Scientific) instead of compound 1c. Example 13 (15 mg) was obtained as a yellow solid. MS: calc'd 435 (MH⁺), measured 435 (MH⁺). ¹H NMR (400 MHz, METHANOL-d₄) δ=8.57 (s, 1H), 8.09 (d, J=7.5 Hz, 1H), 7.39 (d, J=8.4 Hz, 1H), 7.28-7.19 (m, 2H), 6.87 (d, J=7.5 Hz, 1H), 5.21-5.11 (m, 1H), 4.99 (br d, J=15.5 Hz, 1H), 4.13-4.02 (m, 4H), 3.89 (dd, J=7.0, 12.4 Hz, 1H), 3.62-3.53 (m, 2H), 3.51-3.42 (m, 2H), 3.38-3.32 (m, 1H), 3.30-3.26 (m, 2H), 3.01 (s, 6H), 2.07-1.92 (m, 4H), 1.44 (d, J=7.0 Hz, 3H).

### Example 14

### 4-[(dimethylamino)methyl]-1-[(4R)-2-(1,6-dimethylpyrazolo[3,4-b]pyridin-4-yl)-4-methyl-3,4-dihydro-1H-isoquinolin-6-yl]piperidin-4-ol

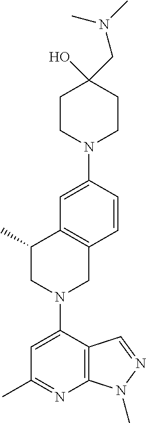 [SMILES: Cc1cc(N2Cc3ccc(N4CCC(O)(CN(C)C)CC4)cc3[C@@H](C)C2)c2cnn(C)c2n1]

The title compound was prepared in analogy to the preparation of compound 1d by using 4-chloro-1,6-dimethyl-pyrazolo[3,4-b]pyridine (CAS: 19867-78-8, Vendor: PharmaBlock) instead of compound 1a and 4-[(dimethylamino)methyl]piperidin-4-ol dihydrochloride (CAS: 695145-47-2, Vendor: J&K Scientific) instead of compound 1c. Example 14 (2 mg) was obtained as a yellow solid. MS: calc'd 449 (MH⁺), measured 449 (MH⁺). ¹H NMR (400 MHz, METHANOL-d₄) δ=8.48 (br s, 1H), 7.33-7.25 (m, 1H), 7.11-7.00 (m, 2H), 6.71 (s, 1H), 5.16-5.04 (m, 1H), 5.00-4.93 (m, 1H), 4.11-3.96 (m, 4H), 3.91-3.80 (m, 1H), 3.52 (br d, J=12.5 Hz, 2H), 3.25 (s, 3H), 3.00 (s, 7H), 2.66 (s, 3H), 2.00-1.79 (m, 5H), 1.42 (d, J=7.0 Hz, 3H).

### Example 15

### (4R)-4-methyl-6-(3-methylpiperazin-1-yl)-2-(1-methylpyrazolo[3,4-b]pyridin-4-yl)-3,4-dihydro-1H-isoquinoline

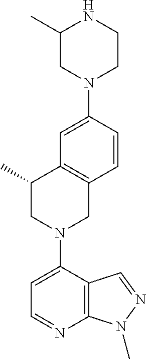 [SMILES: CC1CN(c2ccc3c(c2)[C@@H](C)CN(c2ccnc4c2cnn4C)C3)CCN1]

The title compound was prepared in analogy to the preparation of Example 1 by using tert-butyl 2-methylpiperazine-1-carboxylate (CAS: 120737-78-2, Vendor: Accela) instead of compound 1c. Example 15 (10 mg) was obtained as a yellow solid. MS: calc'd 377 (MH⁺), measured 377 (MH⁺). ¹H NMR (400 MHz, METHANOL-d₄) δ=8.54 (s, 1H), 8.08 (d, J=7.3 Hz, 1H), 7.30 (d, J=8.4 Hz, 1H), 7.03-6.96 (m, 2H), 6.83 (d, J=7.3 Hz, 1H), 5.16-5.06 (m, 1H), 4.94 (br d, J=15.7 Hz, 1H), 4.08 (s, 3H), 4.05-3.99 (m, 1H), 3.84 (br d, J=15.3 Hz, 3H), 3.57-3.46 (m, 2H), 3.35-3.32 (m, 1H), 3.28 (br s, 1H), 3.08-2.98 (m, 1H), 2.81 (dd, J=10.6, 13.1 Hz, 1H), 1.42 (t, J=6.9 Hz, 6H).

### Example 16

### 2-[(4R)-4-methyl-2-(1-methylpyrazolo[3,4-b]pyridin-4-yl)-3,4-dihydro-1H-isoquinolin-6-yl]-2,8-diazaspiro[4.5]decan-3-one

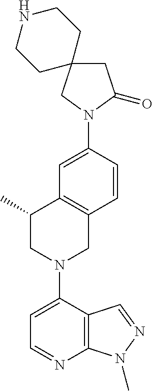 [SMILES: C[C@H]1CN(c2ccnc3c2cnn3C)Cc2ccc(N3CC4(CCNCC4)CC3=O)cc21]

The title compound was prepared according to the following scheme:

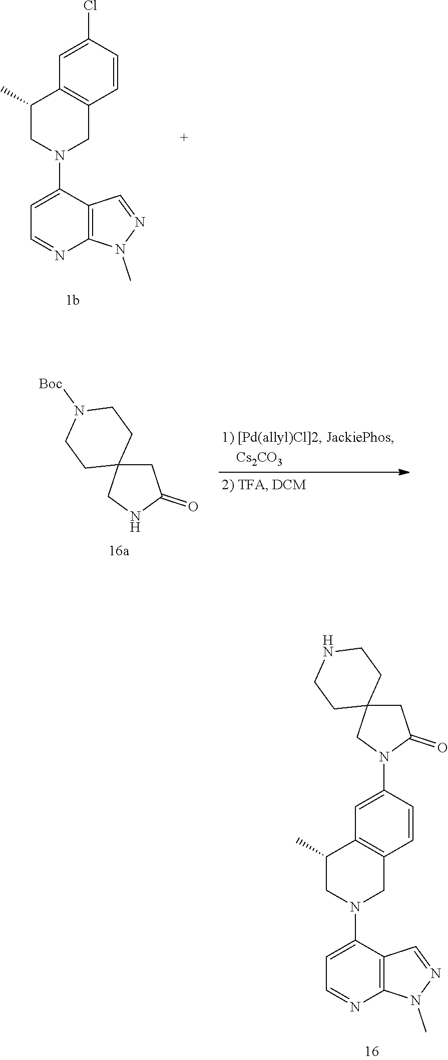 [SMILES: C[C@H]1CN(c2ccnc3c2cnn3C)Cc2ccc(Cl)cc21 ; CC(C)(C)OC(=O)N1CCC2(CC1)CNC(=O)C2 ; C[C@H]1CN(c2ccnc3c2cnn3C)Cc2ccc(N3CC4(CCNCC4)CC3=O)cc21]

A solution of (4R)-6-chloro-4-methyl-2-(1-methylpyrazolo[3,4-b]pyridin-4-yl)-3,4-dihydro-1H-isoquinoline (compound 1b, 70 mg, 224 μmol), tert-butyl 3-oxo-2,8-diazaspiro[4.5]decane-8-carboxylate (compound 16a, CAS: 169206-67-1, Vendor: PharmaBlock, 85 mg, 336 μmol), JackiePhos (CAS: 1160861-60-8, Vendor: J&K Scientific) (18 mg, 23 μmol), allylpalladium(II) chloride dimer (CAS: 12012-95-2, Vendor: Sigma-Aldrich) (9 mg, 23 μmol) and Cs₂CO₃ (219 mg, 671 μmol) in toluene (10 mL) was stirred at 130° C. overnight. After being cooled to room temperature, the reaction mixture was diluted with EA (containing 10% MeOH) (20 mL). The mixture was filtrated and concentrated. The residue was purified by flash column eluting with a gradient of MeOHDCM (0% to 15%) to afford to afford intermediate tert-butyl 3-oxo-2-[(4R)-4-methyl-2-(1-methylpyrazolo[3,4-b]pyridin-4-yl)-3,4-dihydro-1H-isoquinolin-6-yl]-2,8-diazaspiro[4.5]decane-8-carboxylate which was mixed with DCM (1 mL) and TFA (2 mL). The mixture was stirred at room temperature for 1 h and then concentrated. The residue was purified by prep-HPLC to give Example 16 as a light yellow solid (60 mg). MS: calc'd 431 (MH⁺), measured 431 (MH⁺). ¹H NMR (400 MHz, METHANOL-d₄) δ=8.56 (s, 1H), 8.09 (d, J=7.3 Hz, 1H), 7.69-7.69 (m, 1H), 7.53-7.51 (m, 1H), 7.41 (d, J=8.3 Hz, 1H), 6.86-6.86 (m, 1H), 5.18 (d, J=15.8 Hz, 1H), 5.08-4.93 (m, 1H), 4.14-4.04 (m, 4H), 3.95-3.82 (m, 3H), 3.39-3.31 (m, 3H), 3.29-3.20 (m, 2H), 2.67 (s, 2H), 1.98 (br t, J=5.1 Hz, 4H), 1.44 (d, J=6.8 Hz, 3H).

### Example 17

### cis-5-[(4R)-4-methyl-2-(1-methylpyrazolo[3,4-b]pyridin-4-yl)-3,4-dihydro-1H-isoquinolin-6-yl]-1,2,3,3a,6,6a-hexahydropyrrolo[3,4-c]pyrrol-4-one

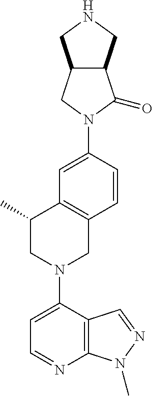

The title compound was prepared in analogy to the preparation of Example 16 by using cis-4-oxo-hexahydro-pyrrolo[3,4-c]pyrrole-2-carboxylic acid tert-butyl ester (CAS: 1251003-89-0, Vendor: PharmaBlock) instead of compound 16a. Example 17 (12 mg) was obtained as a light yellow solid. MS: calc'd 403 (MH⁺), measured 403 (MH⁺). ¹H NMR (400 MHz, METHANOL-d₄) δ=8.30 (s, 1H), 8.12 (d, J=5.9 Hz, 1H), 7.72-7.64 (m, 1H), 7.56-7.48 (m, 1H), 7.37 (d, J=8.4 Hz, 1H), 6.49 (d, J=6.0 Hz, 1H), 4.96 (br s, 1H), 4.77 (s, 1H), 4.33-4.20 (m, 1H), 4.01 (s, 3H), 3.93-3.86 (m, 1H), 3.85-3.75 (m, 2H), 3.72-3.51 (m, 4H), 3.29-3.22 (m, 3H), 1.42 (dd, J=1.8, 6.9 Hz, 3H).

### Example 18

### (4R)-4-methyl-2-(1-methylpyrazolo[3,4-b]pyridin-4-yl)-6-[[(2R)-piperazin-2-yl]methoxy]-3,4-dihydro-1H-isoquinoline

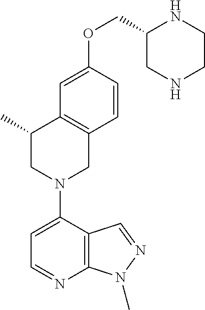 [SMILES: C[C@H]1CN(c2ccnc3c2cnn3C)Cc2ccc(OC[C@H]3CNCCN3)cc21]

The title compound was prepared according to the following scheme:

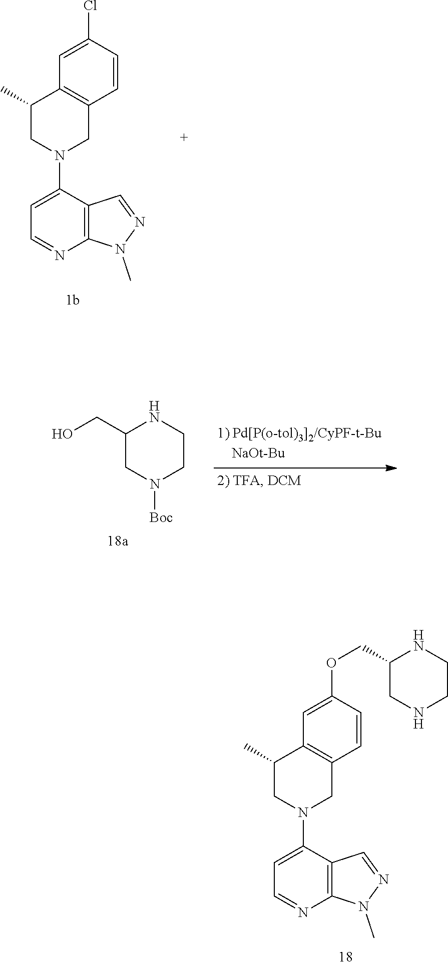 [SMILES: C[C@H]1CN(c2ccnc3c2cnn3C)Cc2ccc(Cl)cc21 ; CC(C)(C)OC(=O)N1CCNC(CO)C1 ; C[C@H]1CN(c2ccnc3c2cnn3C)Cc2ccc(OC[C@H]3CNCCN3)cc21]

To the mixture of (4R)-6-chloro-4-methyl-2-(1-methylpyrazolo[3,4-b]pyridin-4-yl)-3,4-dihydro-1H-isoquinoline (compound 1b, 100 mg, 320 μmol), tert-butyl (R)-3-(hydroxymethyl)piperazine-1-carboxylate (compound 18a, CAS: 278788-66-2, Vendor: Bepharm, 104 mg, 480 μmol), bis(tri-o-tolylphosphine)palladium (CAS: 69861-71-8, Vendor: Alfa Aesar, 19 mg, 26 μmol) and [(R)-1-[(S)-2-(dicyclohexylphosphino)ferrocenyl]ethyl]di-tert-butylphosphine (CAS: 158923-11-6, Vendor: Sigma-Aldrich, 15 mg, 26 μmol) in dioxane (10 mL) was added NaOt-Bu (138 mg, 1.44 mmol). The mixture was charged with nitrogen for 2 minutes, then heated at 120° C. overnight. After being cooled to room temperature, the mixture was quenched by adding saturated ammonium chloride aqueous solution (235 μL, 1.6 mmol). The mixture was stirred for 30 minutes and then dried over Na₂SO₄ and filtered. The filtrate was concentrated and the residue was purified by flash column eluting with a gradient of MeOH/DCM (0% to 20%) to give intermediate tert-butyl (3R)-3-[[(4R)-4-methyl-2-(1-methylpyrazolo[3,4-b]pyridin-4-yl)-3,4-dihydro-1H-isoquinolin-6-yl]oxymethyl]piperazine-1-carboxylate, which was mixed with DCM (1 mL) and TFA (2 mL). The mixture was stirred at rt for 1 h and then concentrated. The residue was purified by prep-HPLC to give Example 18 as a white solid (13 mg). MS: calc'd 393 (MH⁺), measured 393 (MH⁺). ¹H NMR (400 MHz, METHANOL-d₄) δ=8.55 (s, 1H), 8.08 (d, J=7.3 Hz, 1H), 7.35 (d, J=8.4 Hz, 1H), 7.04 (d, J=2.2 Hz, 1H), 6.97 (dd, J=2.5, 8.4 Hz, 1H), 6.85 (d, J=7.3 Hz, 1H), 5.14 (d, J=15.4 Hz, 1H), 4.97 (d, J=15.3 Hz, 1H), 4.36-4.21 (m, 2H), 4.11-4.04 (m, 4H), 3.94-3.80 (m, 2H), 3.76-3.57 (m, 3H), 3.51-3.32 (m, 4H), 1.44 (d, J=7.0 Hz, 3H).

### Example 19

### (4R)-4-methyl-2-(1-methylpyrazolo[3,4-b]pyridin-4-yl)-6-[[(2S)-piperazin-2-yl]methoxy]-3,4-dihydro-1H-isoquinoline

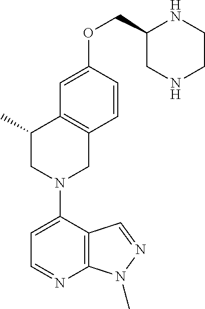 [SMILES: C[C@H]1CN(c2ccnc3c2cnn3C)Cc2ccc(OC[C@@H]3CNCCN3)cc21]

The title compound was prepare in analogy to the preparation o Example 18 by using tert-butyl (S)-3-(hydroxymethyl)piperazine-1-carboxylate (CAS: 314741-40-7, Vendor: Bepharm) instead of compound 18a. Example 19 (32 mg) was obtained as a white solid. MS: calc'd 393 (MH⁺), measured 393 (MH⁺). ¹H NMR (400 MHz, METHANOL-d₄) δ=8.55 (s, 1H), 8.08 (d, J=7.3 Hz, 1H), 7.35 (d, J=8.4 Hz, 1H), 7.04 (s, 1H), 6.98 (br d, J=8.3 Hz, 1H), 6.85 (d, J=7.5 Hz, 1H), 5.14 (d, J=15.4 Hz, 1H), 4.97 (d, J=15.3 Hz, 1H), 4.36-4.20 (m, 2H), 4.12-4.02 (m, 4H), 3.98-3.78 (m, 2H), 3.76-3.58 (m, 3H), 3.48-3.32 (m, 4H), 1.44 (d, J=7.0 Hz, 3H).

### Example 20

### (3R,4R)-4-methoxy-1-[(4R)-4-methyl-2-(1-methylpyrazolo[3,4-b]pyridin-4-yl)-3,4-dihydro-1H-isoquinolin-6-yl]pyrrolidin-3-amine

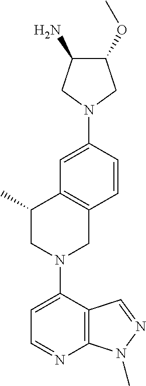 [SMILES: CO[C@@H]1CN(c2ccc3c(c2)[C@@H](C)CN(c2ccnc4c2cnn4C)C3)C[C@H]1N]

The title compound was prepared in analogy to the preparation of Example 1 by using tert-butyl N-[(3R,4R)-4-methoxypyrrolidin-3-yl]carbamate (compound 11b) instead of compound 1c. Example 20 (52 mg) was obtained as a yellow solid. MS: calc'd 393 (MH⁺), measured 393 (MH⁺). ¹H NMR (400 MHz, METHANOL-d₄) δ=8.50 (s, 1H), 8.07 (d, J=7.2 Hz, 1H), 7.23 (d, J=9.0 Hz, 1H), 6.78 (d, J=7.1 Hz, 1H), 6.65-6.57 (m, 2H), 5.05 (d, J=15.2 Hz, 1H), 4.89 (d, J=15.2 Hz, 1H), 4.16-4.10 (m, 1H), 4.07 (s, 3H), 4.00 (dd, J=4.1, 12.3 Hz, 1H), 3.93-3.81 (m, 3H), 3.67 (dd, J=6.3, 10.7 Hz, 1H), 3.50-3.44 (m, 4H), 3.34-3.25 (m, 2H), 1.42 (d, J=7.0 Hz, 3H).

### Example 21

### 2-[(4R)-4-methyl-2-(1-methylpyrazolo[3,4-b]pyridin-4-yl)-3,4-dihydro-1H-isoquinolin-6-yl]-5-oxa-2,8-diazaspiro[3.5]nonane

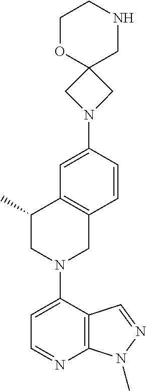 [SMILES: C[C@H]1CN(c2ccnc3c2cnn3C)Cc2ccc(N3CC4(CNCCO4)C3)cc21]

The title compound was prepared in analogy to the preparation of Example 1 by using tert-butyl 5-oxa-2,8-diazaspiro[3.5]nonane-8-carboxylate (CAS: 1251005-61-4, Vendor: PharmaBlock) instead of compound 1c. Example 21 (45 mg) was obtained as a yellow solid. MS: calc'd 405 (MH⁺), measured 405 (MH⁺). ¹H NMR (400 MHz, METHANOL-d₄) δ=8.53 (s, 1H), 8.07 (d, J=7.3 Hz, 1H), 7.22 (d, J=8.2 Hz, 1H), 6.83 (d, J=7.5 Hz, 1H), 6.52-6.44 (m, 2H), 5.12-5.01 (m, 1H), 4.96-4.88 (m, 1H), 4.08 (s, 3H), 4.06-3.99 (m, 3H), 3.95 (dd, J=4.0, 5.8 Hz, 2H), 3.83-3.74 (m, 3H), 3.53 (s, 2H), 3.29-3.23 (m, 3H), 1.41 (d, J=7.0 Hz, 3H).

### Example 22

### (4R)-2-(1-ethylpyrazolo[3,4-b]pyridin-4-yl)-4-methyl-6-piperazin-1-yl-3,4-dihydro-1H-isoquinoline

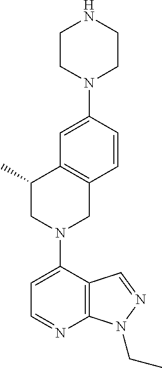 [SMILES: CCn1ncc2c(N3Cc4ccc(N5CCNCC5)cc4[C@@H](C)C3)ccnc21]

The title compound was prepared according to the following scheme:

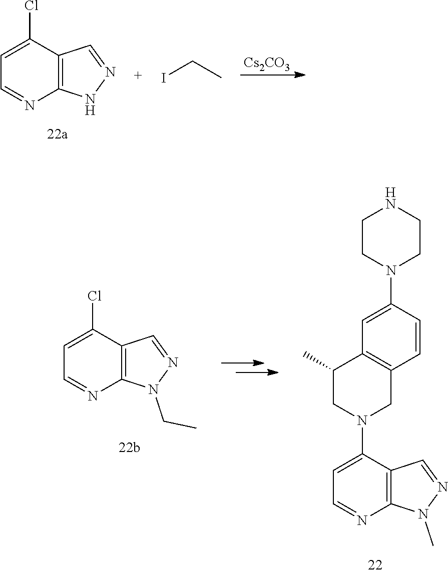

Step 1: preparation of 4-chloro-1-ethyl-pyrazolo[3,4-b]pyridine (compound 22b) To a solution of 4-chloro-1H-pyrazolo[3,4-b]pyridine (compound 22a, CAS: 29274-28-0, Vendor: Bepharm, 500 mg, 3.26 mmol) in DMF (20 mL) was added Cs₂CO₃ (1.59 g, 4.88 mmol). The mixture was cooled down to 0° C., then iodoethane (CAS: 75-03-6, Vendor: J&K Scientific, 609 mg, 3.91 mmol) was added dropwise. After addition, the mixture was slowly warmed to rt and stirred for 5 h. The mixture was poured into ice-water (20 mL), then extracted with EA (20 mL) twice. The organic layer was dried over Na₂SO₄ and concentrated. The residue was purified by flash column eluting with a gradient of EA/PE (10% to 15%) to afford compound 22b (400 mg). MS: calc'd 182 (MH⁺), measured 182 (MH⁺).

### Step 2: preparation of (4R)-2-(1-ethylpyrazolo[3,4-b]pyridin-4-yl)-4-methyl-6-piperazin-1-yl-3,4-dihydro-1H-isoquinoline (Example 22)

The title compound was prepared in analogy to the preparation of Example 1 by using 4-chloro-1-ethyl-pyrazolo[3,4-b]pyridine (compound 22b) instead of compound 1a. Example 22 (45 mg) was obtained as a yellow solid. MS: calc'd 377 (MH⁺), measured 377 (MH⁺). ¹H NMR (400 MHz, METHANOL-d₄) δ=8.58 (s, 1H), 8.07-8.07 (m, 1H), 7.31 (d, J=8.3 Hz, 1H), 7.03-6.96 (m, 2H), 6.86 (d, J=7.5 Hz, 1H), 5.18-5.06 (m, 1H), 4.96 (br d, J=15.5 Hz, 1H), 4.47 (q, J=7.2 Hz, 2H), 4.10-3.98 (m, 1H), 3.95-3.86 (m, 1H), 3.49-3.37 (m, 8H), 3.35-3.31 (m, 1H), 1.51 (t, J=7.3 Hz, 3H), 1.43 (d, J=7.0 Hz, 3H).

### Example 23

### (3R,4R)-4-methoxy-1-[(4R)-2-(1,6-dimethylpyrazolo[3,4-b]pyridin-4-yl)-4-methyl-3,4-dihydro-1H-isoquinolin-6-yl]pyrrolidin-3-amine

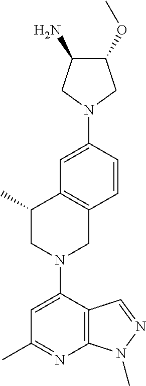 [SMILES: CO[C@@H]1CN(c2ccc3c(c2)[C@@H](C)CN(c2cc(C)nc4c2cnn4C)C3)C[C@H]1N]

The title compound was prepared in analogy to the preparation of Example 1 by using 4-chloro-1,6-dimethyl-pyrazolo[3,4-b]pyridine (CAS: 19867-78-8, Vendor: PharmaBlock) instead of compound 1a and tert-butyl N-[(3R,4R)-4-methoxypyrrolidin-3-yl]carbamate (compound 11b) instead of compound 1c. Example 23 (45 mg) was obtained as a yellow solid. MS: calc'd 407 (MH⁺), measured 407 (MH⁺). ¹H NMR (400 MHz, METHANOL-d₄) δ=8.45 (s, 1H), 7.22 (d, J=8.8 Hz, 1H), 6.68 (s, 1H), 6.64-6.58 (m, 2H), 5.09-4.98 (m, 1H), 4.92-4.87 (m, 1H), 4.16-4.11 (m, 1H), 4.08 (s, 3H), 4.03-3.95 (m, 1H), 3.93-3.78 (m, 3H), 3.67 (dd, J=6.2, 10.6 Hz, 1H), 3.52-3.44 (m, 4H), 3.34-3.31 (m, 1H), 3.29-3.22 (m, 1H), 2.65 (s, 3H), 1.42 (d, J=7.0 Hz, 3H).

### Example 24

### (8aR)-2-[(4R)-4-methyl-2-(1-methylpyrazolo[3,4-b]pyridin-4-yl)-3,4-dihydro-1H-isoquinolin-6-yl]-1,5,6,7,8,8a-hexahydroimidazo[1,5-a]pyrazin-3-one

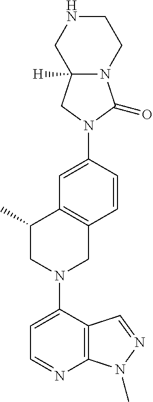 [SMILES: C[C@H]1CN(c2ccnc3c2cnn3C)Cc2ccc(N3C[C@H]4CNCCN4C3=O)cc21]

The title compound was prepared in analogy to the preparation of Example 16 by using tert-butyl (8aS)-3-oxo-1,2,5,6,8,8a-hexahydroimidazo[1,5-a]pyrazine-7-carboxylate (Synthesis procedure refers to patent US20150252057A1) instead of compound 16a. Example 24 (28 mg) was obtained as a yellow solid. MS: calc'd 418 (MH⁺), measured 418 (MH⁺). ¹H NMR (400 MHz, METHANOL-d₄) δ=8.56 (s, 1H), 8.09 (d, J=7.5 Hz, 1H), 7.65 (d, J=2.0 Hz, 1H), 7.48-7.43 (m, 1H), 7.40-7.34 (m, 1H), 6.86 (d, J=7.5 Hz, 1H), 5.16 (br d, J=15.6 Hz, 1H), 5.01 (s, 1H), 4.18-4.03 (m, 7H), 3.87 (dd, J=7.2, 12.5 Hz, 1H), 3.73-3.66 (m, 1H), 3.55 (dd, J=2.6, 12.3 Hz, 1H), 3.42 (dd, J=3.2, 12.5 Hz, 1H), 3.36-3.32 (m, 1H), 3.30-3.27 (m, 1H), 3.17-3.07 (m, 2H), 1.44 (d, J=7.0 Hz, 3H).

### Example 25

### (8aS)-2-[(4R)-4-methyl-2-(1-methylpyrazolo[3,4-b]pyridin-4-yl)-3,4-dihydro-1H-isoquinolin-6-yl]-1,5,6,7,8,8a-hexahydroimidazo[1,5-a]pyrazin-3-one

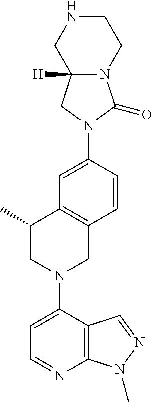 [SMILES: C[C@H]1CN(c2ccnc3c2cnn3C)Cc2ccc(N3C[C@@H]4CNCCN4C3=O)cc21]

The title compound was prepared in analogy to the preparation of Example 16 by using tert-butyl (8aR)-3-oxo-1,2,5,6,8,8a-hexahydroimidazo[1,5-a]pyrazine-7-carboxylate (Synthesis procedure refers to patent US20150252057A1) instead of compound 16a. Example 25 (29 mg) was obtained as a yellow solid. MS: calc'd 418 (MH⁺), measured 418 (MH⁺). ¹H NMR (400 MHz, METHANOL-d₄) δ=8.57 (s, 1H), 8.09 (d, J=7.5 Hz, 1H), 7.65 (d, J=2.0 Hz, 1H), 7.46 (dd, J=2.3, 8.4 Hz, 1H), 7.40-7.35 (m, 1H), 6.86 (d, J=7.5 Hz, 1H), 5.16 (br d, J=15.5 Hz, 1H), 5.06-4.92 (m, 1H), 4.17-4.04 (m, 7H), 3.87 (dd, J=7.2, 12.3 Hz, 1H), 3.75-3.67 (m, 1H), 3.55 (dd, J=2.7, 12.3 Hz, 1H), 3.42 (dd, J=3.2, 12.5 Hz, 1H), 3.37-3.32 (m, 1H), 3.30-3.26 (m, 1H), 3.15-3.05 (m, 2H), 1.44 (d, J=7.0 Hz, 3H).

### Example 26

### 3-[(4R)-4-methyl-2-(1-methylpyrazolo[3,4-b]pyridin-4-yl)-3,4-dihydro-1H-isoquinolin-6-yl]-1-oxa-3,8-diazaspiro[4.5]decan-2-one

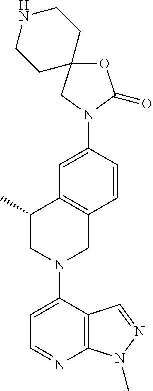 [SMILES: C[C@H]1CN(c2ccnc3c2cnn3C)Cc2ccc(N3CC4(CCNCC4)OC3=O)cc21]

The title compound was prepared in analogy to the preparation of Example 16 by using tert-butyl 2-oxo-1-oxa-3,8-diazaspiro[4.5]decane-8-carboxylate (CAS: 169206-55-7, Vendor: Bepharm) instead of compound 16a. Example 26 (20 mg) was obtained as a light yellow solid. MS: calc'd 433 (MH⁺), measured 433 (MH⁺). ¹H NMR (400 MHz, METHANOL-d₄) δ=8.55 (s, 1H), 8.09 (d, J=7.2 Hz, 1H), 7.65 (d, J=2.1 Hz, 1H), 7.52-7.47 (m, 1H), 7.44-7.39 (m, 1H), 6.84 (d, J=7.3 Hz, 1H), 5.23-5.12 (m, 1H), 5.06-4.96 (m, 1H), 4.11-4.01 (m, 6H), 3.88 (dd, J=7.0, 12.4 Hz, 1H), 3.48-3.33 (m, 5H), 2.35-2.24 (m, 2H), 2.22-2.13 (m, 2H), 1.45 (d, J=7.0 Hz, 3H).

### Example 27

### (4R)-6-(3,8-diazabicyclo[3.2.1]octan-8-yl)-4-methyl-2-(1-methylpyrazolo[3,4-b]pyridin-4-yl)-3,4-dihydro-1H-isoquinoline

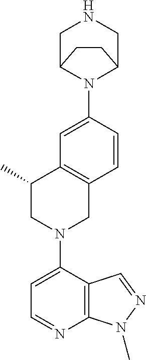 [SMILES: C[C@H]1CN(c2ccnc3c2cnn3C)Cc2ccc(N3C4CCC3CNC4)cc21]

The title compound was prepared in analogy to the preparation of Example 1 by using tert-butyl 3,8-diazabicyclo[3.2.1]octane-3-carboxylate (CAS: 201162-53-0, Vendor: Bepharm) instead of compound 1c. Example 27 (64 mg) was obtained as a yellow solid. MS: calc'd 389 (MH⁺), measured 389 (MH⁺). ¹H NMR (400 MHz, METHANOL-d₄) δ=8.55 (s, 1H), 8.08 (d, J=7.3 Hz, 1H), 7.29 (d, J=8.3 Hz, 1H), 6.94-6.83 (m, 3H), 5.15-5.06 (m, 1H), 4.94 (br d, J=15.5 Hz, 1H), 4.49 (br d, J=2.1 Hz, 2H), 4.09 (s, 3H), 4.06-3.99 (m, 1H), 3.89 (dd, J=6.8, 12.4 Hz, 1H), 3.35 (br d, J=12.7 Hz, 2H), 3.30-3.26 (m, 1H), 3.13 (br d, J=12.6 Hz, 2H), 2.32-2.23 (m, 2H), 2.11-2.04 (m, 2H), 1.43 (d, J=7.0 Hz, 3H).

### Example 28

### 1-[1-[(4R)-4-methyl-2-(1-methylpyrazolo[3,4-b]pyridin-4-yl)-3,4-dihydro-1H-isoquinolin-6-yl]-4-piperidyl]azetidin-3-amine

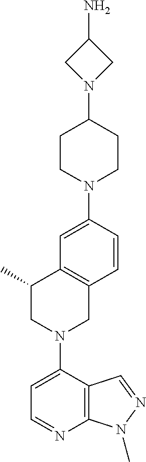 [SMILES: C[C@H]1CN(c2ccnc3c2cnn3C)Cc2ccc(N3CCC(N4CC(N)C4)CC3)cc21]

The title compound was prepared in analogy to the preparation of Example 11 by using tert-butyl N-(azetidin-3-yl)carbamate (CAS: 91188-13-5, Vendor: Bepharm) instead of compound 11b. Example 28 (8 mg) was obtained as a yellow solid. MS: calc'd 432 (MH⁺), measured 432 (MH⁺). ¹H NMR (400 MHz, METHANOL-d₄) δ=8.55 (s, 1H), 8.07 (d, J=7.5 Hz, 1H), 7.27 (d, J=8.3 Hz, 1H), 7.04-6.93 (m, 2H), 6.85 (d, J=7.3 Hz, 1H), 5.16-5.06 (m, 1H), 4.94 (br d, J=14.9 Hz, 1H), 4.60-4.51 (m, 2H), 4.44-4.34 (m, 3H), 4.09 (s, 3H), 4.07-3.99 (m, 1H), 3.91-3.81 (m, 3H), 3.50-3.41 (m, 1H), 3.30-3.25 (m, 1H), 2.84 (br t, J=11.6 Hz, 2H), 2.13 (br d, J=12.3 Hz, 2H), 1.66 (dq, J=4.1, 11.7 Hz, 2H), 1.42 (d, J=7.0 Hz, 3H).

### Example 29

### trans-4-amino-1-[1-[(4R)-4-methyl-2-(1-methylpyrazolo[3,4-b]pyridin-4-yl)-3,4-dihydro-1H-isoquinolin-6-yl]-4-piperidyl]piperidin-3-ol

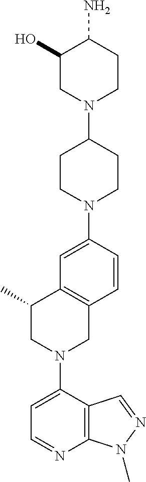 [SMILES: C[C@H]1CN(c2ccnc3c2cnn3C)Cc2ccc(N3CCC(N4CC[C@@H](N)[C@H](O)C4)CC3)cc21]

The title compound was prepared in analogy to the preparation of Example 11 by using trans-tert-butyl N-[3-hydroxy-4-piperidyl]carbamate (CAS: 859854-66-3, Vendor: PharmaBlock) instead of compound 11b. Example 29 (8 mg) was obtained as a yellow solid. MS: calc'd 476 (MH⁺), measured 476 (MH⁺). ¹H NMR (400 MHz, METHANOL-d₄) δ=8.55 (s, 1H), 8.07 (d, J 15=7.3 Hz, 1H), 7.27 (d, J=8.3 Hz, 1H), 7.03-6.95 (m, 2H), 6.85 (d, J=7.5 Hz, 1H), 5.11 (br d, J=15.9 Hz, 1H), 4.96 (br s, 1H), 4.09 (s, 3H), 4.06-3.82 (m, 5H), 3.75-3.62 (m, 2H), 3.48 (tt, J=3.6, 12.1 Hz, 1H), 3.30 (br s, 3H), 3.07-2.97 (m, 1H), 2.87 (br t, J=11.7 Hz, 2H), 2.36 (br dd, J=3.6, 14.4 Hz, 1H), 2.29-2.17 (m, 2H), 2.05-1.86 (m, 3H), 1.42 (d, J=7.0 Hz, 3H).

### Example 30

### (4R)-2-(1,6-dimethylpyrazolo[3,4-b]pyridin-4-yl)-4-methyl-6-[[(2R)-piperazin-2-yl]methoxy]-3,4-dihydro-1H-isoquinoline

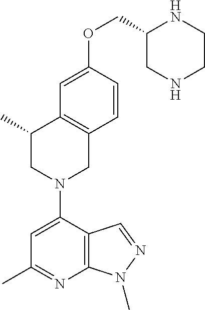 [SMILES: Cc1cc(N2Cc3ccc(OC[C@H]4CNCCN4)cc3[C@@H](C)C2)c2cnn(C)c2n1]

The title compound was prepared in analogy to the preparation of Example 18 by using 4-chloro-1,6-dimethyl-pyrazolo[3,4-b]pyridine (CAS: 19867-78-8, Vendor: PharmaBlock) instead of compound 1a. Example 30 (29 mg) was obtained as a white solid. MS: calc'd 407 (MH⁺), measured 407 (MH⁺). ¹H NMR (400 MHz, METHANOL-d₄) δ=8.48 (s, 1H), 7.34 (d, J=8.4 Hz, 1H), 7.03 (d, J=2.3 Hz, 1H), 6.97 (dd, J=2.4, 8.4 Hz, 1H), 6.71 (s, 1H), 5.11 (br d, J=14.9 Hz, 1H), 5.00-4.90 (m, 1H), 4.35-4.21 (m, 2H), 4.12-4.01 (m, 4H), 3.97-3.87 (m, 1H), 3.84-3.69 (m, 2H), 3.68-3.58 (m, 2H), 3.51-3.26 (m, 4H), 2.66 (s, 3H), 1.43 (d, J=7.0 Hz, 3H).

### Example 31

### (4R)-2-(1,6-dimethylpyrazolo[3,4-b]pyridin-4-yl)-4-methyl-6-[[(2S)-piperazin-2-yl]methoxy]-3,4-dihydro-1H-isoquinoline

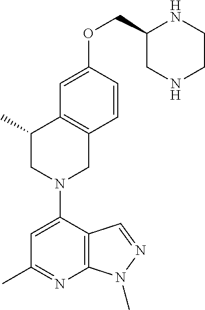 [SMILES: Cc1cc(N2Cc3ccc(OC[C@@H]4CNCCN4)cc3[C@@H](C)C2)c2cnn(C)c2n1]

The title compound was prepared in analogy to the preparation of Example 18 by using 4-chloro-1,6-dimethyl-pyrazolo[3,4-b]pyridine (CAS: 19867-78-8, Vendor: PharmaBlock) instead of compound 1a, tert-butyl (S)-3-(hydroxymethyl)piperazine-1-carboxylate (CAS: 314741-40-7, Vendor: Bepharm) instead of compound 18a. Example 31 (23 mg) was obtained as a white solid. MS: calc'd 407 (MH⁺), measured 407 (MH⁺). ¹H NMR (400 MHz, METHANOL-d₄) δ=8.48 (s, 1H), 7.34 (d, J=8.4 Hz, 1H), 7.04 (d, J=2.3 Hz, 1H), 6.97 (dd, J=2.5, 8.4 Hz, 1H), 6.71 (s, 1H), 5.16-5.07 (m, 1H), 5.00-4.90 (m, 1H), 4.36-4.22 (m, 2H), 4.12-4.01 (m, 4H), 3.98-3.89 (m, 1H), 3.85-3.72 (m, 2H), 3.69-3.60 (m, 2H), 3.50-3.32 (m, 4H), 2.66 (s, 3H), 1.43 (d, J=6.8 Hz, 3H).

### Example 33

### (4R)-4-methyl-2-(1-methylpyrazolo[3,4-b]pyridin-4-yl)-6-(4-pyrrolidin-3-ylsulfonylpiperazin-1-yl)-3,4-dihydro-1H-isoquinoline

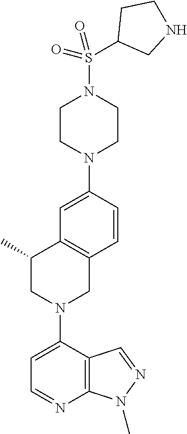 [SMILES: C[C@H]1CN(c2ccnc3c2cnn3C)Cc2ccc(N3CCN(S(=O)(=O)C4CCNC4)CC3)cc21]

The title compound was prepared according to the following scheme:

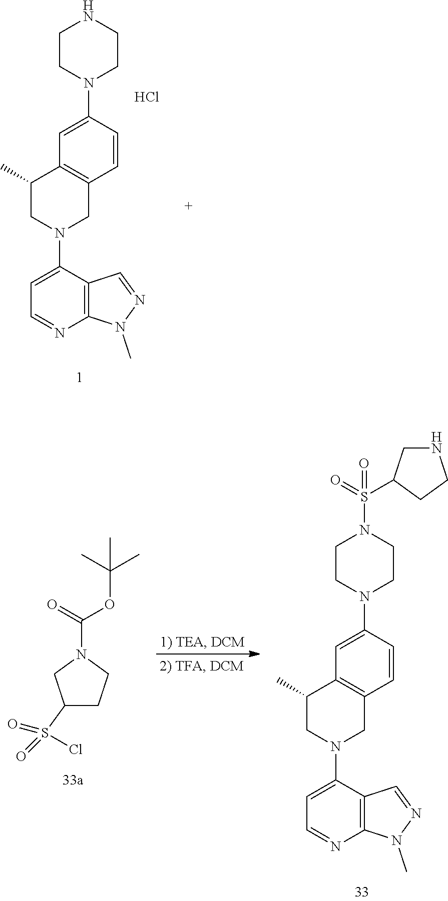

The mixture of (4R)-4-methyl-2-(1-methylpyrazolo[3,4-b]pyridin-4-yl)-6-piperazin-1-yl-3,4-dihydro-1H-isoquinoline hydrochloride (HCl salt of Example 1, 30 mg, 75 μmol), tert-butyl 3-chlorosulfonylpyrrolidine-1-carboxylate (compound 33a, CAS: 935845-20-8, Vendor: Bide Pharmatech, 30 mg, 113 μmol) and TEA (52 μL, 376 μmol) in DCM (10 mL) was stirred at rt for 2 h. After concentration, the mixture was purified by flash column eluting with a gradient of EA(with 10% MeOH)/PE (0% to 100%) to afford a yellow oil which was re-dissolved in DCM (10 mL) and TFA (2 mL) and stirred at rt for 2 h. After concentration, the mixture was purified by reversed flash column eluting with a gradient of ACN/Water (with 0.05% TFA) (0 to 30%) to give Example 33 (16 mg) as a yellow solid. MS: calc'd 496 (MH⁺), measured 496 (MH⁺). ¹H NMR (400 MHz, METHANOL-d₄) δ=8.55 (s, 1H), 8.07 (d, J=7.3 Hz, 1H), 7.27 (d, J=8.3 Hz, 1H), 6.98 (s, 1H), 6.95 (d, J=8.0 Hz, 1H), 6.85 (d, J=7.3 Hz, 1H), 5.11 (br d, J=15.2 Hz, 1H), 4.96 (br s, 1H), 4.18 (td, J=6.9, 13.8 Hz, 1H), 4.09 (s, 3H), 4.08-4.01 (m, 1H), 3.88 (dd, J=6.8, 12.3 Hz, 1H), 3.74-3.62 (m, 2H), 3.57-3.47 (m, 6H), 3.45-3.34 (m, 1H), 3.29-3.26 (m, 4H), 2.52-2.42 (m, 2H), 1.42 (d, J=7.0 Hz, 3H).

### Example 34

### 6-[(4R)-4-methyl-2-(1-methylpyrazolo[3,4-b]pyridin-4-yl)-3,4-dihydro-1H-isoquinolin-6-yl]-2,6-diazaspiro[3.4]octan-7-one

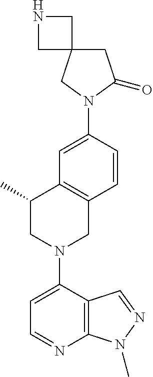 [SMILES: C[C@H]1CN(c2ccnc3c2cnn3C)Cc2ccc(N3CC4(CNC4)CC3=O)cc21]

The title compound was prepared in analogy to the preparation of Example 16 by using tert-butyl 6-oxo-2,7-diazaspiro[3.4]octane-2-carboxylatecarboxylate (CAS: 1234616-51-3, Vendor: PharmaBlock) instead of compound 16a. Example 34 (84 mg) was obtained as a light yellow solid. MS: calc'd 403 (MH⁺), measured 403 (MH⁺). ¹H NMR (400 MHz, METHANOL-d₄) δ=8.57 (s, 1H), 8.09 (d, J=7.5 Hz, 1H), 7.66 (d, J=1.8 Hz, 1H), 7.53-7.48 (m, 1H), 7.45-7.40 (m, 1H), 6.87 (d, J=7.5 Hz, 1H), 5.23-5.13 (m, 1H), 5.03 (br s, 1H), 4.29-4.19 (m, 6H), 4.14-4.05 (m, 4H), 3.88 (dd, J=7.2, 12.5 Hz, 1H), 3.38-3.32 (m, 1H), 3.04 (s, 2H), 1.44 (d, J=6.8 Hz, 3H).

### Example 35

### 3-[(4R)-4-methyl-2-(1-methylpyrazolo[3,4-b]pyridin-4-yl)-3,4-dihydro-1H-isoquinolin-6-yl]-1-oxa-3,7-diazaspiro[4.4]nonan-2-one

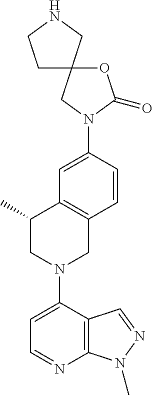 [SMILES: C[C@H]1CN(c2ccnc3c2cnn3C)Cc2ccc(N3CC4(CCNC4)OC3=O)cc21]

The title compound was prepared in analogy to the preparation of Example 16 by using tert-butyl 2-oxo-1-oxa-3,7-diazaspiro[4.4]nonane-7-carboxylate (CAS: 1642899-83-9, Vendor: Sigma-Aldrich) instead of compound 16a. Example 35 (8 mg) was obtained as a light yellow solid. MS: calc'd 419 (MH⁺), measured 419 (MH⁺). ¹H NMR (400 MHz, METHANOL-d₄) δ=8.57 (s, 1H), 8.09 (d, J=7.3 Hz, 1H), 7.66 (d, J=1.8 Hz, 1H), 7.53-7.47 (m, 1H), 7.46-7.41 (m, 1H), 6.86 (d, J=7.3 Hz, 1H), 5.22-5.13 (m, 1H), 5.01 (br d, J=15.5 Hz, 1H), 4.35-4.27 (m, 2H), 4.12-4.04 (m, 4H), 3.89 (dd, J=7.2, 12.4 Hz, 1H), 3.81 (dd, J=1.7, 13.1 Hz, 1H), 3.68-3.61 (m, 1H), 3.58-3.52 (m, 2H), 3.40-3.34 (m, 1H), 2.65-2.55 (m, 1H), 2.45-2.35 (m, 1H), 1.45 (d, J=7.0 Hz, 3H).

### Example 36

### (3R,4R)-4-amino-3-methyl-1-[(4R)-4-methyl-2-(1-methylpyrazolo[3,4-b]pyridin-4-yl)-3,4-dihydro-1H-isoquinolin-6-yl]pyrrolidin-3-ol

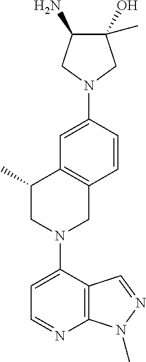 [SMILES: C[C@H]1CN(c2ccnc3c2cnn3C)Cc2ccc(N3C[C@@H](N)[C@](C)(O)C3)cc21]

The title compound was prepared in analogy to the preparation of Example 1 by using tert-butyl N-[(3R,4R)-4-hydroxy-4-methyl-pyrrolidin-3-yl]carbamate (Synthesis procedure refers to patent WO2019233941A1) instead of compound 1c. Example 36 (41 mg) was obtained as a yellow solid. MS: calc'd 393 (MH⁺), measured 393 (MH⁺). ¹H NMR (400 MHz, METHANOL-d₄) δ=8.53 (s, 1H), 8.06 (d, J=7.5 Hz, 1H), 7.22 (d, J=9.0 Hz, 1H), 6.82 (d, J=7.3 Hz, 1H), 6.58-6.53 (m, 2H), 5.12-5.02 (m, 1H), 4.92 (br s, 1H), 4.08 (s, 3H), 4.05-3.99 (m, 1H), 3.85 (td, J=5.3, 10.6 Hz, 2H), 3.67 (dd, J=2.9, 5.8 Hz, 1H), 3.59 (d, J=10.4 Hz, 1H), 3.49 (dd, J=2.9, 10.7 Hz, 1H), 3.36 (d, J=10.4 Hz, 1H), 3.30-3.24 (m, 1H), 1.49 (s, 3H), 1.42 (d, J=7.0 Hz, 3H).

### Example 37

### (4R)-4-methyl-6-[4-[(1-methylimidazol-4-yl)methyl]piperazin-1-yl]-2-(1-methylpyrazolo[3,4-b]pyridin-4-yl)-3,4-dihydro-1H-isoquinoline

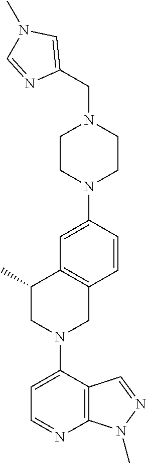 [SMILES: C[C@H]1CN(c2ccnc3c2cnn3C)Cc2ccc(N3CCN(Cc4cn(C)cn4)CC3)cc21]

The title compound was prepared according to the following scheme:

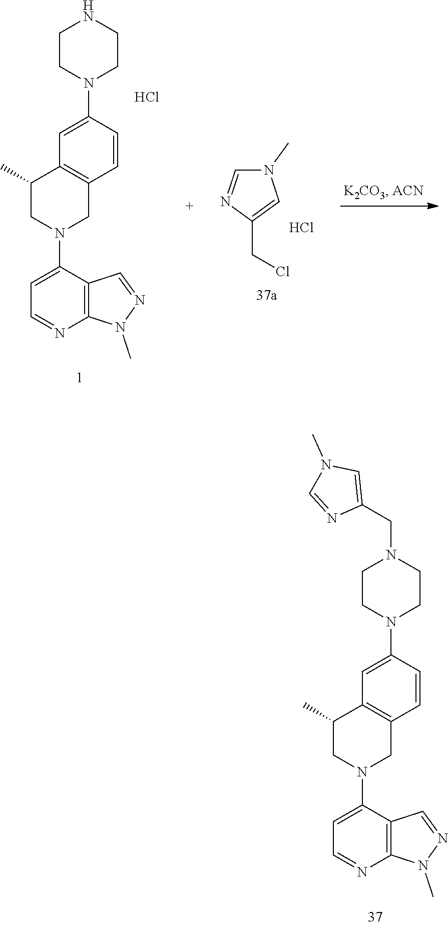

Step 1: preparation of(4R)-4-methyl-6-[4-[(1-methylimidazol-4-yl)methyl]piperazin-1-yl]-2-(1-methylpyrazolo[3,4-b]pyridin-4-yl)-3,4-dihydro-1H-isoquinoline (Example 37) The mixture of(4R)-4-methyl-2-(1-methylpyrazolo[3,4-b]pyridin-4-yl)-6-piperazin-1-yl-3,4-dihydro-1H-isoquinoline hydrochloride (HCl salt of Example 1, 50 mg, 125 μmol), 4-(chloromethyl)-1-methyl-imidazole hydrochloride (CAS: 17289-30-4, Vendor: Bepharm, 21 mg, 125 μmol) and potassium carbonate (87 mg, 627 μmol) in acetonitrile (5 mL) was heated to 80° C. overnight. After being cooled to room temperature, the solid was filtered off and washed with EA (10 mL) for two times. The combined mixture was concentrated and purified by prep-HPLC to give Example 37 (1.5 mg) as a light yellow solid. MS: calc'd 457 (MH⁺), measured 457 (MH⁺). ¹H NMR (400 MHz, METHANOL-d₄) δ=8.27 (s, 1H), 8.09 (d, J=6.0 Hz, 1H), 7.55 (s, 1H), 7.17 (d, J=8.2 Hz, 1H), 7.05 (s, 1H), 6.90-6.85 (m, 2H), 6.45 (d, J=6.0 Hz, 1H), 4.84-4.82 (m, 1H), 4.74-4.66 (m, 1H), 3.99 (s, 3H), 3.85-3.73 (m, 2H), 3.72 (s, 3H), 3.56 (s, 2H), 3.23-3.15 (m, 5H), 2.74-2.64 (m, 4H), 1.38 (d, J=7.0 Hz, 3H).

### Example 38

### N,N-dimethyl-2-[4-[(4R)-4-methyl-2-(1-methylpyrazolo[3,4-b]pyridin-4-yl)-3,4-dihydro-1H-isoquinolin-6-yl]piperazin-1-yl]ethanamine

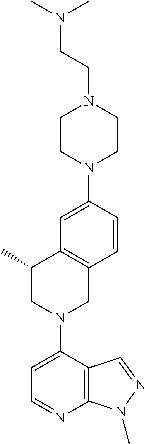 [SMILES: C[C@H]1CN(c2ccnc3c2cnn3C)Cc2ccc(N3CCN(CCN(C)C)CC3)cc21]

The title compound was prepared in analogy to the preparation of Example 37 by using 2-bromo-N,N-dimethyl-ethanamine hydrobromide (CAS: 2862-39-7, Vendor: Accela) instead of 4-(chloromethyl)-1-methyl-imidazole hydrochloride (compound 37a). Example 38 (2.5 mg) was obtained as a light yellow solid. MS: calc'd 434 (MH⁺), measured 434 (MH⁺). ¹H NMR (400 MHz, METHANOL-d₄) δ=8.27 (s, 1H), 8.09 (d, J=5.9 Hz, 1H), 7.18 (d, J=8.2 Hz, 1H), 6.91-6.86 (m, 2H), 6.44 (d, J=6.0 Hz, 1H), 4.84 (d, J=15.0 Hz, 1H), 4.69 (d, J=14.9 Hz, 1H), 3.99 (s, 3H), 3.86-3.71 (m, 2H), 3.20 (br d, J=5.1 Hz, 5H), 2.71-2.64 (m, 4H), 2.62-2.54 (m, 4H), 2.31 (s, 6H), 1.38 (d, J=7.0 Hz, 3H).

### Example 39

### (4R)-6-(4,4-dimethylpiperazin-4-ium-1-yl)-4-methyl-2-(1-methylpyrazolo[3,4-b]pyridin-4-yl)-3,4-dihydro-1H-isoquinoline chloride

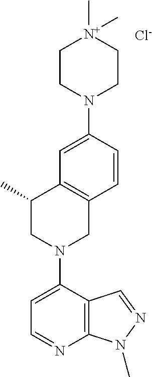 [SMILES: C[C@H]1CN(c2ccnc3c2cnn3C)Cc2ccc(N3CC[N+](C)(C)CC3)cc21 ; [Cl-]]

The title compound was prepared in analogy to the preparation of Example 37 by using iodomethane (CAS: 74-88-4, Vendor: Sinopharm) instead of 4-(chloromethyl)-1-methyl-imidazole hydrochloride (compound 37a) and changing the reaction temperature from 80° C. to rt. Example 39 (3 mg) was obtained as a yellow solid. MS: calc'd 391 (M-Cl⁻), measured 391 (M−Cl⁻). ¹H NMR (400 MHz, METHANOL-d₄) δ=8.70-8.40 (m, 1H), 8.08 (br s, 1H), 7.40-7.25 (m, 1H), 7.11-6.98 (m, 2H), 6.95-6.83 (m, 1H), 5.33-5.10 (m, 1H), 5.05-4.89 (m, 1H), 4.30-4.04 (m, 4H), 3.96-3.82 (m, 2H), 3.69-3.63 (m, 3H), 3.60 (br d, J=5.5 Hz, 3H), 3.40-3.23 (m, 7H), 3.20-3.04 (m, 1H), 1.53-1.36 (m, 3H).

### Example 40

### (4R)-4-methyl-2-(1-methylpyrazolo[3,4-b]pyridin-4-yl)-6-(2-piperazin-1-ylethoxy)-3,4-dihydro-1H-isoquinoline

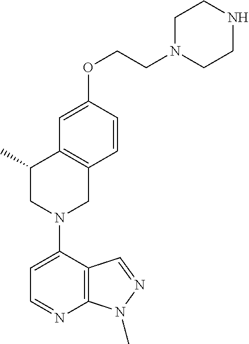 [SMILES: C[C@H]1CN(c2ccnc3c2cnn3C)Cc2ccc(OCCN3CCNCC3)cc21]

The title compound was prepared in analogy to the preparation of Example 18 by using tert-butyl 4-(2-hydroxyethyl)piperazine-1-carboxylate (CAS: 77279-24-4, Vendor: Bepharm) instead of compound 18a. Example 40 (22 mg) was obtained as a light yellow solid. MS: calc'd 407 (MH⁺), measured 407 (MH⁺). ¹H NMR (400 MHz, METHANOL-d₄) δ=8.56 (s, 1H), 8.08 (d, J=7.3 Hz, 1H), 7.32 (d, J=8.4 Hz, 1H), 6.98 (d, J=2.4 Hz, 1H), 6.93 (dd, J=2.6, 8.4 Hz, 1H), 6.86 (d, J=7.5 Hz, 1H), 5.19-5.08 (m, 1H), 5.03-4.92 (m, 1H), 4.31 (t, J=5.0 Hz, 2H), 4.12-4.03 (m, 4H), 3.83 (dd, J=7.5, 12.3 Hz, 1H), 3.49-3.42 (m, 4H), 3.35-3.31 (m, 7H), 1.43 (d, J=7.0 Hz, 3H).

### Example 41

### (4R)-4-methyl-2-(1-methylpyrazolo[3,4-b]pyridin-4-yl)-6-[1-(4-piperidyl)azetidin-3-yl]oxy-3,4-dihydro-1H-isoquinoline

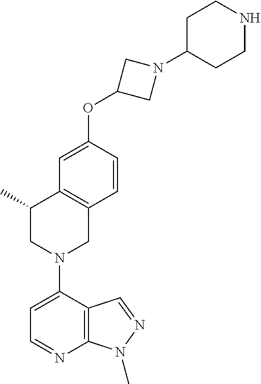 [SMILES: C[C@H]1CN(c2ccnc3c2cnn3C)Cc2ccc(OC3CN(C4CCNCC4)C3)cc21]

The title compound was prepared according to the following scheme:

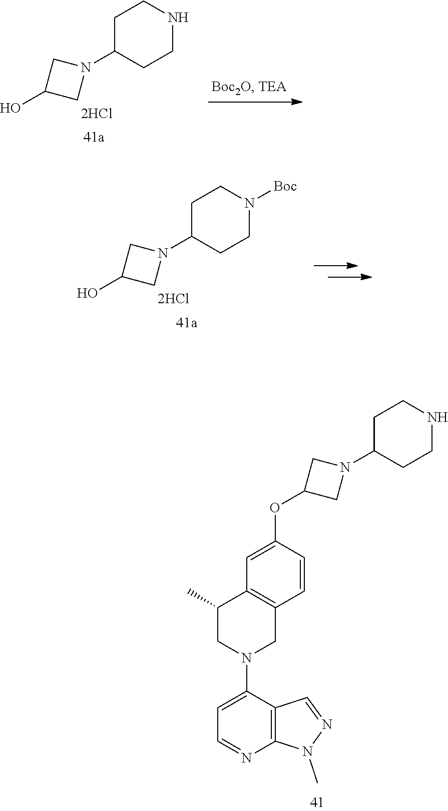 [SMILES: OC1CN(C2CCNCC2)C1 ; CC(C)(C)OC(=O)N1CCC(N2CC(O)C2)CC1 ; C[C@H]1CN(c2ccnc3c2cnn3C)Cc2ccc(OC3CN(C4CCNCC4)C3)cc21]

### Step 1: preparation of tert-butyl 4-(3-hydroxyazetidin-1-yl)piperidine-1-carboxylate (compound 41b)

To a solution of 1-(piperidin-4-yl)azetidin-3-ol dihydrochloride (compound 41a, CAS: 1537465-19-2, Vendor: Bepharm, 200 mg, 873 μmol) in DCM (4 mL) and Water (0.4 mL) was added triethylamine (600 μL, 4.3 mmol) and di-tert butyl dicarbonate (243 μL, 1.05 mmol) at rt. The result solution was stirred at rt overnight. The reaction mixture was concentrated in vacuo. The residue was purified by flash column eluting with a gradient of EA(with 10% MeOH)/PE (80% to 100%) to afford compound 41b (200 mg). MS: calc'd 257 (MH⁺), measured 257 (MH⁺).

### Step 2: preparation of (4R)-4-methyl-2-(1-methylpyrazolo[3,4-b]pyridin-4-yl)-6-[1-(4-piperidyl)azetidin-3-yl]oxy-3,4-dihydro-1H-isoquinoline (Example 41)

The title compound was prepared in analogy to the preparation of Example 18 by using tert-butyl 4-(3-hydroxyazetidin-1-yl)piperidine-1-carboxylate (compound 41b) instead of compound 18a. Example 41 (13 mg) was obtained as a green solid. MS: calc'd 433 (MH⁺), measured 433 (MH⁺). ¹H NMR (400 MHz, METHANOL-d₄) δ=8.55 (s, 1H), 8.08 (d, J=7.5 Hz, 1H), 7.39-7.33 (m, 1H), 6.89 (d, J=2.4 Hz, 1H), 6.87-6.80 (m, 2H), 5.25-5.17 (m, 1H), 5.16-5.10 (m, 1H), 4.96 (br d, J=15.7 Hz, 1H), 4.73 (br dd, J=6.4, 11.9 Hz, 2H), 4.33 (br d, J=9.4 Hz, 2H), 4.12-4.02 (m, 4H), 3.85 (dd, J=7.3, 12.4 Hz, 1H), 3.65-3.54 (m, 3H), 3.36-3.32 (m, 1H), 3.07 (dt, J=2.6, 13.1 Hz, 2H), 2.31 (br d, J=13.3 Hz, 2H), 1.82-1.71 (m, 2H), 1.43 (d, J=7.0 Hz, 3H).

### Example 42

### (4R)-4-methyl-2-(1-methylpyrazolo[3,4-b]pyridin-4-yl)-6-[[(7R,8aS)-1,2,3,4,6,7,8,8a-octahydropyrrolo[1,2-a]pyrazin-7-yl]oxy]-3,4-dihydro-1H-isoquinoline

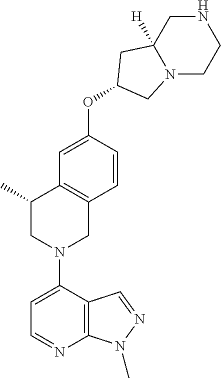 [SMILES: C[C@H]1CN(c2ccnc3c2cnn3C)Cc2ccc(O[C@@H]3C[C@H]4CNCCN4C3)cc21]

The title compound was prepared in analogy to the preparation of Example 18 by using tert-butyl (7R,8aS)-7-hydroxy-octahydropyrrolo[1,2-a]piperazine-2-carboxylate (CAS: 1204603-42-8, Vendor: PharmaBlock) instead of compound 18a. Example 42 (25 mg) was obtained as a white solid. MS: calc'd 419 (MH⁺), measured 419 (MH⁺). ¹H NMR (400 MHz, METHANOL-d₄) δ=8.56 (s, 1H), 8.08 (d, J=7.5 Hz, 1H), 7.31 (d, J=8.4 Hz, 1H), 6.89 (d, J=2.3 Hz, 1H), 6.88-6.82 (m, 2H), 5.13 (br d, J=15.2 Hz, 1H), 5.08-5.00 (m, 1H), 4.97-4.93 (m, 1H), 4.15-4.02 (m, 4H), 3.88-3.79 (m, 2H), 3.57 (br d, J=11.0 Hz, 1H), 3.42 (br d, J=12.8 Hz, 1H), 3.37-3.27 (m, 2H), 3.18 (dt, J=3.7, 12.4 Hz, 1H), 2.98-2.82 (m, 2H), 2.73 (dt, J=3.1, 12.3 Hz, 1H), 2.63 (dd, J=4.4, 10.4 Hz, 1H), 2.21-2.12 (m, 1H), 2.09-1.99 (m, 1H), 1.42 (d, J=7.0 Hz, 3H).

### Example 43

### (4R)-4-methyl-2-(1-methylpyrazolo[3,4-b]pyridin-4-yl)-6-[[(2R)-1-methylpiperazin-2-yl]methoxy]-3,4-dihydro-1H-isoquinoline

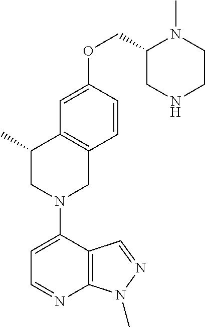 [SMILES: C[C@H]1CN(c2ccnc3c2cnn3C)Cc2ccc(OC[C@H]3CNCCN3C)cc21]

The title compound was prepared according to the following scheme:

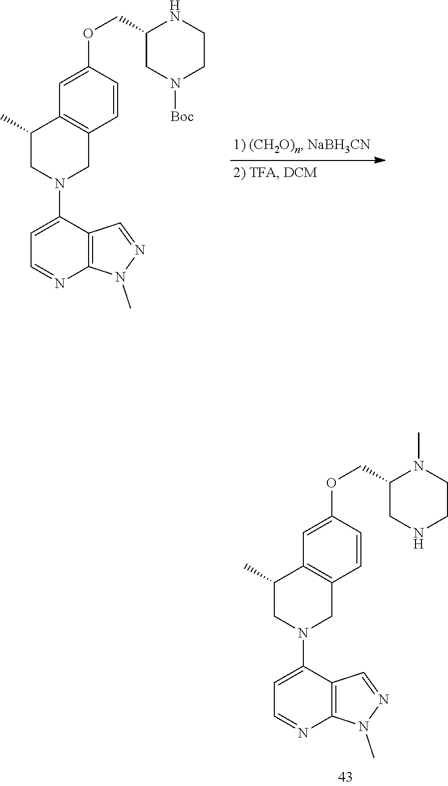 [SMILES: C[C@H]1CN(c2ccnc3c2cnn3C)Cc2ccc(OC[C@H]3CN(C(=O)OC(C)(C)C)CCN3)cc21 ; C[C@H]1CN(c2ccnc3c2cnn3C)Cc2ccc(OC[C@H]3CNCCN3C)cc21]

To a solution of tert-butyl (3R)-3-[[(4R)-4-methyl-2-(1-methylpyrazolo[3,4-b]pyridin-4-yl)-3,4-dihydro-1H-isoquinolin-6-yl]oxymethyl]piperazine-1-carboxylate (Intermediate from Example 18, 90 mg, 183 μmol) in ethanol (15 mL) was added paraformaldehyde (CAS: 30525-89-4, Vendor: TCI chemicals, 28 mg, 933 μmol) and 3 drops of acetic acid. The mixture was sealed and stirred at 50° C. for 1 h, then NaBH₃CN (CAS: 25895-60-7, Vendor: Accela, 23 mg, 365 μmol) was added. The mixture was stirred at 50° C. overnight. After being cooled to room temperature, the mixture was quenched by adding saturated NaHCO₃ (10 mL) aqueous solution. The mixture was extracted by EA (10 mL) three times and the combined organic phase was dried over Na₂SO₄, filtered and concentrated. The residue was purified by flash column eluting with a gradient of EA(with 10% MeOH)/PE (50% to 100%) to afford intermediate tert-butyl (3R)-4-methyl-3-[[(4R)-4-methyl-2-(1-methylpyrazolo[3,4-b]pyridin-4-yl)-3,4-dihydro-1H-isoquinolin-6-yl]oxymethyl]piperazine-1-carboxylate, which was mixed with DCM (1 mL) and TFA (2 mL). The mixture was stirred at rt for 30 minutes, then concentrated. The residue was purified by reversed-phase flash column to give Example 43 (5 mg) as a light yellow solid. MS: calc'd 407 (MH⁺), measured 407 (MH⁺). ¹H NMR (400 MHz, METHANOL-d₄) δ=8.56 (s, 1H), 8.08 (d, J=7.3 Hz, 1H), 7.35 (d, J=8.4 Hz, 1H), 7.04 (d, J=2.3 Hz, 1H), 6.97 (dd, J=2.6, 8.4 Hz, 1H), 6.86 (d, J=7.3 Hz, 1H), 5.14 (br d, J=15.4 Hz, 1H), 4.97 (br d, J=15.7 Hz, 1H), 4.44 (dd, J=3.4, 11.2 Hz, 1H), 4.32-4.25 (m, 1H), 4.12-4.03 (m, 4H), 3.83 (dd, J=7.5, 12.4 Hz, 1H), 3.77-3.68 (m, 1H), 3.63-3.42 (m, 5H), 3.35-3.31 (m, 1H), 3.26-3.16 (m, 1H), 2.83 (s, 3H), 1.44 (d, J=7.0 Hz, 3H).

### Example 44

### (4R)-4-methyl-2-(1-methylpyrazolo[3,4-b]pyridin-4-yl)-6-[[(2S)-1-methylpiperazin-2-yl]methoxy]-3,4-dihydro-1H-isoquinoline

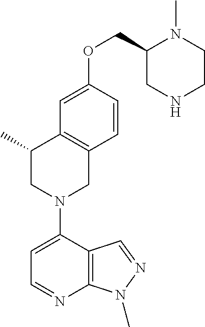 [SMILES: C[C@H]1CN(c2ccnc3c2cnn3C)Cc2ccc(OC[C@@H]3CNCCN3C)cc21]

The title compound was prepared in analogy to the preparation of Example 43 by using tert-butyl (3S)-3-[[(4R)-4-methyl-2-(1-methylpyrazolo[3,4-b]pyridin-4-yl)-3,4-dihydro-1H-isoquinolin-6-yl]oxymethyl]piperazine-1-carboxylate (Intermediate from Example 19) instead of tert-butyl (3R)-3-[[(4R)-4-methyl-2-(1-methylpyrazolo[3,4-b]pyridin-4-yl)-3,4-dihydro-1H-isoquinolin-6-yl]oxymethyl]piperazine-1-carboxylate (Intermediate from Example 18). Example 44 (18 mg) was obtained as a light yellow solid. MS: calc'd 407 (MH⁺), measured 407 (MH⁺). ¹H NMR (400 MHz, METHANOL-d₄) δ=8.56 (s, 1H), 8.08 (d, J=7.3 Hz, 1H), 7.34 (d, J=8.3 Hz, 1H), 7.03 (d, J=2.4 Hz, 1H), 6.96 (dd, J=2.6, 8.4 Hz, 1H), 6.86 (d, J=7.5 Hz, 1H), 5.14 (br d, J=15.3 Hz, 1H), 4.96 (br d, J=15.4 Hz, 1H), 4.40 (dd, J=3.9, 11.0 Hz, 1H), 4.26 (dd, J=2.8, 10.9 Hz, 1H), 4.11-4.05 (m, 4H), 3.83 (dd, J=7.5, 12.3 Hz, 1H), 3.68 (br d, J=12.3 Hz, 1H), 3.55 (br d, J=13.3 Hz, 1H), 3.51-3.32 (m, 5H), 3.16-3.03 (m, 1H), 2.76 (s, 3H), 1.44 (d, J=7.0 Hz, 3H).

### Example 45

### (2S)-2-[[(4R)-4-methyl-2-(1-methylpyrazolo[3,4-b]pyridin-4-yl)-3,4-dihydro-1H-isoquinolin-6-yl]oxymethyl]piperazine-1-carbaldehyde

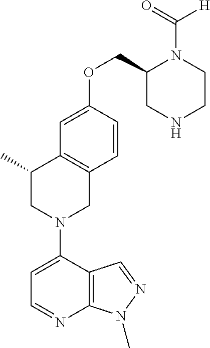 [SMILES: C[C@H]1CN(c2ccnc3c2cnn3C)Cc2ccc(OC[C@@H]3CNCCN3C=O)cc21]

The title compound was obtained during prep-HPLC purification (0.1% formic acid as additive) of Example 19. Example 45 (10 mg) was obtained as a light yellow solid. MS: calc'd 421 (MH⁺), measured 421 (MH⁺). ¹H NMR (400 MHz, METHANOL-d₄) δ=8.72-8.40 (m, 1H), 8.15 (d, J=9.2 Hz, 1H), 8.09 (br d, J=6.0 Hz, 1H), 7.36 (br s, 1H), 7.08 (br s, 1H), 7.04-6.98 (m, 1H), 6.88 (br d, J=7.3 Hz, 1H), 5.33-4.91 (m, 2H), 4.56-4.43 (m, 1H), 4.42-4.35 (m, 1H), 4.28 (dd, J=6.1, 10.8 Hz, 1H), 4.16-4.06 (m, 4H), 4.04-3.94 (m, 1H), 3.89-3.68 (m, 2H), 3.64-3.50 (m, 2H), 3.36-3.31 (m, 2H), 3.24-3.16 (m, 1H), 1.45 (br d, J=6.2 Hz, 3H).

### Example 46

### trans-4-amino-1-[(4R)-4-methyl-2-(1-methylpyrazolo[3,4-b]pyridin-4-yl)-3,4-dihydro-1H-isoquinolin-6-yl]pyrrolidin-3-ol

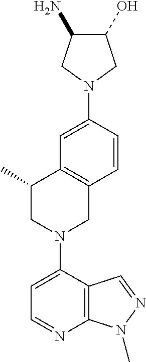 [SMILES: C[C@H]1CN(c2ccnc3c2cnn3C)Cc2ccc(N3C[C@@H](N)[C@H](O)C3)cc21]

The title compound was prepared in analogy to the preparation of Example 1 by using trans-tert-butyl N-(4-hydroxypyrrolidin-3-yl)carbamate (CAS: 870632-89-6, Vendor: PharmaBlock) instead of compound 1c. Example 46 (10 mg) was obtained as a yellow solid. MS: calc'd 379 (MH⁺), measured 379 (MH⁺). ¹H NMR (400 MHz, METHANOL-d₄) δ=8.53 (s, 1H), 8.06 (d, J=7.3 Hz, 1H), 7.23 (d, J=8.9 Hz, 1H), 6.82 (d, J=7.5 Hz, 1H), 6.63-6.56 (m, 2H), 5.13-5.03 (m, 1H), 4.95-4.87 (m, 1H), 4.50-4.43 (m, 1H), 4.08 (s, 3H), 4.02 (br dd, J=3.8, 12.1 Hz, 1H), 3.88-3.80 (m, 2H), 3.78-3.73 (m, 2H), 3.48-3.42 (m, 1H), 3.30-3.22 (m, 2H), 1.43 (d, J=7.0 Hz, 3H).

### Example 47

### trans-4-amino-1-[(4R)-2-(1,6-dimethylpyrazolo[3,4-b]pyridin-4-yl)-4-methyl-3,4-dihydro-1H-isoquinolin-6-yl]pyrrolidin-3-ol

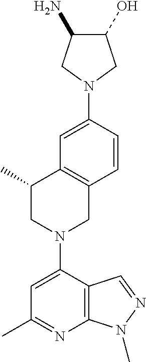 [SMILES: Cc1cc(N2Cc3ccc(N4C[C@@H](N)[C@H](O)C4)cc3[C@@H](C)C2)c2cnn(C)c2n1]

The title compound was prepared in analogy to the preparation of Example 1 by using 4-chloro-1,6-dimethyl-pyrazolo[3,4-b]pyridine (CAS: 19867-78-8, Vendor: PharmaBlock) instead of compound 1a, trans-tert-butyl N-(4-hydroxypyrrolidin-3-yl)carbamate (CAS: 870632-89-6, Vendor: PharmaBlock) instead of compound 1c. Example 47 (10 mg) was obtained as a yellow solid. MS: calc'd 393 (MH⁺), measured 393 (MH⁺). ¹H NMR (400 MHz, METHANOL-d₄) δ=8.45 (br s, 1H), 7.22 (d, J=8.8 Hz, 1H), 6.68 (s, 1H), 6.62-6.56 (m, 2H), 5.09-4.98 (m, 1H), 4.94-4.88 (m, 1H), 4.49-4.44 (m, 1H), 4.08 (s, 3H), 4.04-3.93 (m, 1H), 3.83 (dd, J=5.9, 10.5 Hz, 2H), 3.78-3.72 (m, 2H), 3.48-3.41 (m, 1H), 3.33-3.22 (m, 2H), 2.65 (s, 3H), 1.42 (d, J=7.0 Hz, 3H).

### Example 48

### 2-[(4R)-2-(1,6-dimethylpyrazolo[3,4-b]pyridin-4-yl)-4-methyl-3,4-dihydro-1H-isoquinolin-6-yl]-5-oxa-2,8-diazaspiro[3.5]nonane

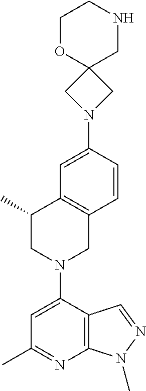 [SMILES: Cc1cc(N2Cc3ccc(N4CC5(CNCCO5)C4)cc3[C@@H](C)C2)c2cnn(C)c2n1]

The title compound was prepared in analogy to the preparation of Example 1 by using 4-chloro-1,6-dimethyl-pyrazolo[3,4-b]pyridine (CAS: 19867-78-8, Vendor: PharmaBlock) instead of compound 1a, tert-butyl 5-oxa-2,8-diazaspiro[3.5]nonane-8-carboxylate (CAS: 1251005-61-4, Vendor: PharmaBlock) instead of compound 1c. Example 48 (27 mg) was obtained as a yellow solid. MS: calc'd 419 (MH⁺), measured 419 (MH⁺). ¹H NMR (400 MHz, METHANOL-d₄) δ=8.36 (s, 1H), 7.11 (d, J=8.2 Hz, 1H), 6.59 (s, 1H), 6.45-6.35 (m, 2H), 4.94 (br d, J=14.7 Hz, 1H), 4.85-4.78 (m, 1H), 4.01-3.89 (m, 6H), 3.87-3.82 (m, 2H), 3.73-3.64 (m, 3H), 3.43 (s, 2H), 3.19-3.12 (m, 3H), 2.55 (s, 3H), 1.31 (d, J=7.0 Hz, 3H).

### Example 49

### [4-[(4R)-4-methyl-2-(1-methylpyrazolo[3,4-b]pyridin-4-yl)-3,4-dihydro-1H-isoquinolin-6-yl]piperazin-2-yl]methanol

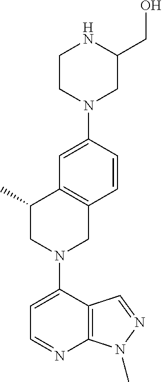 [SMILES: C[C@H]1CN(c2ccnc3c2cnn3C)Cc2ccc(N3CCNC(CO)C3)cc21]

The title compound was prepared in analogy to the preparation of Example 1 by using tert-butyl 2-(hydroxymethyl)piperazine-1-carboxylate (CAS: 205434-75-9, Vendor: Accela) instead of compound 1c. Example 49 (7 mg) was obtained as a white solid. MS: calc'd 393 (MH⁺), measured 393 (MH⁺). ¹H NMR (400 MHz, METHANOL-d₄) δ=8.28 (s, 1H), 8.09 (d, J=5.9 Hz, 1H), 7.19 (d, J=8.1 Hz, 1H), 6.97-6.83 (m, 2H), 6.45 (d, J=5.9 Hz, 1H), 4.96-4.89 (m, 1H), 4.75-4.67 (m, 1H), 4.00 (s, 3H), 3.89-3.74 (m, 2H), 3.68-3.50 (m, 4H), 3.23-3.09 (m, 2H), 3.03-2.93 (m, 2H), 2.80-2.70 (m, 1H), 2.48 (t, J=11.0 Hz, 1H), 1.39 (d, J=6.8 Hz, 3H).

### Example 50

### [(2R)-4-[(4R)-4-methyl-2-(1-methylpyrazolo[3,4-b]pyridin-4-yl)-3,4-dihydro-1H-isoquinolin-6-yl]piperazin-2-yl]methanol

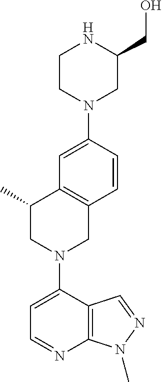 [SMILES: C[C@H]1CN(c2ccnc3c2cnn3C)Cc2ccc(N3CCN[C@@H](CO)C3)cc21]

The title compound was prepared in analogy to the preparation of Example 1 by using tert-butyl (2R)-2-(hydroxymethyl)piperazine-1-carboxylate (CAS: 169448-87-7, Vendor: Bepharm) instead of compound 1c. Example 50 (10 mg) was obtained as a white solid. MS: calc'd 393 (MH⁺), measured 393 (MH⁺). ¹H NMR (400 MHz, METHANOL-d₄) δ=8.28 (s, 1H), 8.10 (d, J=5.9 Hz, 1H), 7.20 (d, J=8.2 Hz, 1H), 6.96-6.86 (m, 2H), 6.46 (d, J=6.0 Hz, 1H), 4.88-4.83 (m, 1H), 4.76-4.66 (m, 1H), 4.00 (s, 3H), 3.88-3.72 (m, 2H), 3.68-3.53 (m, 4H), 3.24-3.14 (m, 2H), 3.08-2.96 (m, 2H), 2.77 (dt, J=2.9, 11.6 Hz, 1H), 2.52 (dd, J=10.7, 11.4 Hz, 1H), 1.39 (d, J=7.0 Hz, 3H).

### Example 51

### [(2S)-4-[(4R)-4-methyl-2-(1-methylpyrazolo[3,4-b]pyridin-4-yl)-3,4-dihydro-1H-isoquinolin-6-yl]piperazin-2-yl]methanol

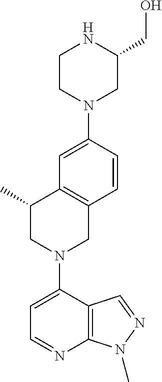 [SMILES: C[C@H]1CN(c2ccnc3c2cnn3C)Cc2ccc(N3CCN[C@H](CO)C3)cc21]

The title compound was prepared in analogy to the preparation of Example 1 by using tert-butyl (2S)-2-(hydroxymethyl)piperazine-1-carboxylate (CAS: 1030377-21-9, Vendor: Accela) instead of compound 1c. Example 51 (5 mg) was obtained as a white solid. MS: calc'd 393 (MH⁺), measured 393 (MH⁺). ¹H NMR (400 MHz, METHANOL-d₄) δ=8.28 (s, 1H), 8.10 (d, J=5.9 Hz, 1H), 7.20 (d, J=8.2 Hz, 1H), 6.95-6.87 (m, 2H), 6.46 (d, J=6.0 Hz, 1H), 4.88-4.83 (m, 1H), 4.75-4.67 (m, 1H), 4.00 (s, 3H), 3.88-3.76 (m, 2H), 3.68-3.56 (m, 4H), 3.23-3.14 (m, 2H), 3.08-2.98 (m, 2H), 2.83-2.74 (m, 1H), 2.57-2.49 (m, 1H), 1.39 (d, J=7.0 Hz, 3H).

### Example 52

### 2-[(4R)-4-methyl-2-(1-methylpyrazolo[3,4-b]pyridin-4-yl)-3,4-dihydro-1H-isoquinolin-6-yl]-2,7-diazaspiro[4.4]nonan-3-one

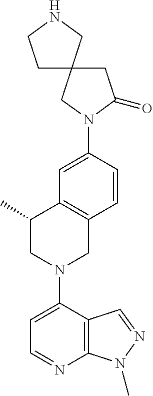 [SMILES: C[C@H]1CN(c2ccnc3c2cnn3C)Cc2ccc(N3CC4(CCNC4)CC3=O)cc21]

The title compound was prepared in analogy to the preparation of Example 16 by using tert-butyl 8-oxo-2,7-diazaspiro[4.4]nonane-2-carboxylate (CAS: 1251009-03-6, Vendor: PharmaBlock) instead of compound 16a. Example 52 (5 mg) was obtained as a white solid. MS: calc'd 417 (MH⁺), measured 417 (MH⁺). ¹H NMR (400 MHz, METHANOL-d₄) δ=8.29 (s, 1H), 8.11 (d, J=5.7 Hz, 1H), 7.60 (s, 1H), 7.45 (br d, J=8.3 Hz, 1H), 7.40-7.30 (m, 1H), 6.48 (d, J=5.9 Hz, 1H), 4.95 (br s, 1H), 4.82-4.74 (m, 1H), 4.07-3.94 (m, 5H), 3.89 (br dd, J=4.0, 12.1 Hz, 1H), 3.82-3.72 (m, 1H), 3.53-3.40 (m, 4H), 3.28-3.21 (m, 1H), 2.88-2.68 (m, 2H), 2.30-2.15 (m, 2H), 1.42 (br d, J=6.8 Hz, 3H).

### Example 53 & Example 54

### (5S)-2-[(4R)-4-methyl-2-(1-methylpyrazolo[3,4-b]pyridin-4-yl)-3,4-dihydro-1H-isoquinolin-6-yl]-2,7-diazaspiro[4.4]nonan-3-one & (5R)-2-[(4R)-4-methyl-2-(1-methylpyrazolo[3,4-b]pyridin-4-yl)-3,4-dihydro-1H-isoquinolin-6-yl]-2,7-diazaspiro[4,4]nonan-3-one

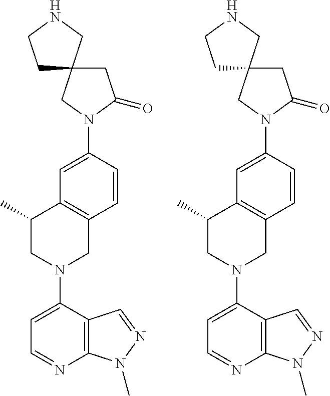 [SMILES: C[C@H]1CN(c2ccnc3c2cnn3C)Cc2ccc(N3C[C@@]4(CCNC4)CC3=O)cc21 ; C[C@H]1CN(c2ccnc3c2cnn3C)Cc2ccc(N3C[C@]4(CCNC4)CC3=O)cc21]

The title compounds were separated by chiral SFC of Example 52 (Gradient: 50% Methanol (0.1% NH₃H₂O) in CO₂, Column: AY, 250×20 mm, 5 μm).

Example 53 (faster eluting, 36 mg) was obtained as a green solid. MS: calc'd 417 (MH⁺), measured 417 (MH⁺). ¹H NMR (400 MHz, METHANOL-d₄) δ=8.54 (s, 1H), 8.09 (d, J=7.2 Hz, 1H), 7.66 (d, J=2.0 Hz, 1H), 7.52-7.46 (m, 1H), 7.44-7.39 (m, 1H), 6.82 (d, J=7.2 Hz, 1H), 5.21-5.12 (m, 1H), 5.03-4.95 (m, 1H), 4.10-4.02 (m, 5H), 3.99-3.94 (m, 1H), 3.87 (dd, J=7.1, 12.5 Hz, 1H), 3.52-3.47 (m, 2H), 3.46-3.38 (m, 2H), 3.37-3.32 (m, 1H), 2.87-2.80 (m, 1H), 2.78-2.70 (m, 1H), 2.31-2.17 (m, 2H), 1.44 (d, J=6.8 Hz, 3H).

Example 54 (slower eluting, 46 mg) was obtained as a yellow solid. MS: calc'd 417 (MH⁺), measured 417 (MH⁺). ¹H NMR (400 MHz, METHANOL-d₄) δ=8.57 (s, 1H), 8.09 (d, J=7.3 Hz, 1H), 7.67 (d, J=2.0 Hz, 1H), 7.49 (d, J=2.2 Hz, 1H), 7.45-7.39 (m, 1H), 6.86 (d, J=7.3 Hz, 1H), 5.23-5.13 (m, 1H), 5.07-4.97 (m, 1H), 4.11-4.05 (m, 4H), 4.05-3.94 (m, 2H), 3.89 (dd, J=7.2, 12.5 Hz, 1H), 3.52-3.47 (m, 2H), 3.43 (d, J=7.3 Hz, 2H), 3.38-3.32 (m, 1H), 2.87-2.71 (m, 2H), 2.30-2.16 (m, 2H), 1.45 (d, J=7.0 Hz, 3H).

### Example 55

### cis-2-[(4R)-4-methyl-2-(1-methylpyrazolo[3,4-b]pyridin-4-yl)-3,4-dihydro-1H-isoquinolin-6-yl]-3a,4,5,6,7,7a-hexahydro-1H-pyrrolo[3,4-c]pyridin-3-one

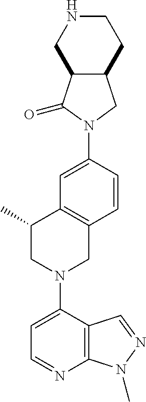

The title compound was prepared in analogy to the preparation of Example 16 by using cis-tert-butyl 3-oxo-2,3a,4,6,7,7a-hexahydro-1H-pyrrolo[3,4-c]pyridine-5-carboxylate (compound 55a) instead of compound 16a. Compound 55a was prepared in analogy to the preparation of compound 41b by using cis-1,2,3a,4,5,6,7,7a-octahydropyrrolo[3,4-c]pyridin-3-one (CAS: 868551-69-3, Vendor: PharmaBlock) instead of compound 41a. Example 55 (10 mg) was obtained as a white solid. MS: calc'd 417 (MH⁺), measured 417 (MH⁺). ¹H NMR (400 MHz, METHANOL-d₄) δ=8.29 (s, 1H), 8.11 (d, J=5.9 Hz, 1H), 7.67 (dd, J=1.8, 11.7 Hz, 1H), 7.47 (dt, J=2.1, 8.1 Hz, 1H), 7.33 (d, J=8.4 Hz, 1H), 6.48 (d, J=5.9 Hz, 1H), 4.96-4.90 (m, 1H), 4.80-4.70 (m, 1H), 4.08-3.97 (m, 4H), 3.92-3.85 (m, 1H), 3.81-3.72 (m, 1H), 3.50-3.38 (m, 2H), 3.27-3.19 (m, 1H), 2.97-2.86 (m, 2H), 2.74-2.52 (m, 3H), 1.89-1.78 (m, 1H), 1.50-1.36 (m, 4H).

### Example 56 & Example 57

### (3aS,7aS)-2-[(4R)-4-methyl-2-(1-methylpyrazolo[3,4-b]pyridin-4-yl)-3,4-dihydro-1H-isoquinolin-6-yl]-3a,4,5,6,7,7a-hexahydro-1H-pyrrolo[3,4-c]pyridin-3-one & (3aR,7aR)-2-[(4R)-4-methyl-2-(1-methylpyrazolo[3,4-b]pyridin-4-yl)-3,4-dihydro-1H-isoquinolin-6-yl]-3a,4,5,6,7,7a-hexahydro-1H-pyrrolo[3,4-c]pyridin-3-one

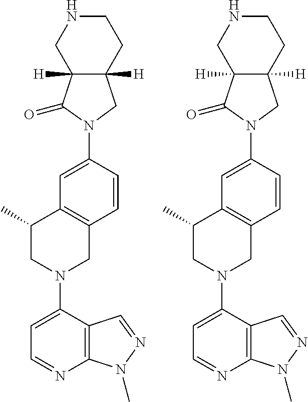

The title compounds were separated by chiral SFC of Example 55 (Gradient: 40% Isopropanol (0.1% NH₃H₂O) in CO₂, Column: OD, 250×20 mm, 5 μm).

Example 56 (faster eluting, 16 mg) was obtained as a green solid. MS: calc'd 417 (MH⁺), measured 417 (MH⁺). ¹H NMR (400 MHz, METHANOL-d₄) δ=8.57 (s, 1H), 8.10 (d, J=7.2 Hz, 1H), 7.74 (d, J=2.0 Hz, 1H), 7.58 (dd, J=2.2, 8.3 Hz, 1H), 7.43 (d, J=8.4 Hz, 1H), 6.86 (d, J=7.3 Hz, 1H), 5.23-5.14 (m, 1H), 5.01 (br d, J=15.7 Hz, 1H), 4.15 (dd, J=5.5, 10.0 Hz, 1H), 4.11-4.03 (m, 4H), 3.90 (dd, J=7.1, 12.5 Hz, 1H), 3.81 (d, J=13.4 Hz, 1H), 3.57 (d, J=10.0 Hz, 1H), 3.40-3.31 (m, 3H), 3.18-3.11 (m, 1H), 3.03 (dt, J=2.7, 13.0 Hz, 1H), 2.82 (qd, J=6.1, 11.9 Hz, 1H), 2.16-2.05 (m, 1H), 1.75-1.61 (m, 1H), 1.46 (d, J=7.0 Hz, 3H).

Example 57 (slower eluting, 26 mg) was obtained as a green solid. MS: calc'd 417 (MH⁺), measured 417 (MH⁺). ¹H NMR (400 MHz, METHANOL-d₄) δ=8.57 (s, 1H), 8.10 (d, J=7.3 Hz, 1H), 7.76 (d, J=1.8 Hz, 1H), 7.56 (dd, J=2.1, 8.3 Hz, 1H), 7.43 (d, J=8.4 Hz, 1H), 6.86 (d, J=7.3 Hz, 1H), 5.24-5.13 (m, 1H), 5.02 (br d, J=15.7 Hz, 1H), 4.15 (dd, J=5.4, 10.0 Hz, 1H), 4.13-4.04 (m, 4H), 3.94-3.77 (m, 2H), 3.56 (d, J=10.0 Hz, 1H), 3.41-3.31 (m, 3H), 3.14 (br t, J=6.0 Hz, 1H), 3.03 (dt, J=2.6, 13.0 Hz, 1H), 2.82 (qd, J=6.0, 11.9 Hz, 1H), 2.18-2.06 (m, 1H), 1.77-1.62 (m, 1H), 1.45 (d, J=7.0 Hz, 3H).

### Example 58

### 1-[(8S)-6-(1,6-dimethylpyrazolo[3,4-b]pyridin-4-yl)-8-methyl-7,8-dihydro-5H-1,6-naphthyridin-2-yl]piperidin-4-amine

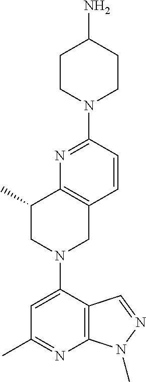 [SMILES: Cc1cc(N2Cc3ccc(N4CCC(N)CC4)nc3[C@@H](C)C2)c2cnn(C)c2n1]

The title compound was prepared in analogy to the preparation of Example 1 by using 4-chloro-1,6-dimethyl-pyrazolo[3,4-b]pyridine (CAS: 19867-78-8, Vendor: PharmaBlock) instead of compound 1a, Intermediate C instead of Intermediate A, tert-butyl N-(4-piperidyl)carbamate (CAS: 73874-95-0, Vendor: Accela) instead of compound 1c and changing the Buchwald coupling reaction condition from RuPhos Pd G2, Cs₂CO₃, 1,4-dioxane to Pd₂(dba)₃, BINAP, NaOt-Bu, toluene. Example 58 (15 mg) was obtained as a light yellow solid. MS: calc'd 392 (MH⁺), measured 392 (MH⁺). ¹H NMR (400 MHz, METHANOL-d₄) δ=8.18 (s, 1H), 7.46 (d, J=8.7 Hz, 1H), 6.76 (d, J=8.7 Hz, 1H), 6.39 (s, 1H), 4.69 (d, J=10.9 Hz, 2H), 4.49 (br d, J=12.8 Hz, 2H), 4.04-3.92 (m, 4H), 3.74 (br d, J=7.0 Hz, 1H), 3.37 (br s, 1H), 3.13 (br d, J=4.5 Hz, 1H), 2.94 (br t, J=13.0 Hz, 2H), 2.54 (s, 3H), 2.06 (br d, J=10.9 Hz, 2H), 1.63 (br dd, J=3.8, 12.1 Hz, 2H), 1.41 (d, J=7.0 Hz, 3H).

### Example 59

### 4-methyl-1-[(8S)-6-(1,6-dimethylpyrazolo[3,4-b]pyridin-4-yl)-8-methyl-7,8-dihydro-5H-1,6-naphthyridin-2-yl]piperidin-4-amine

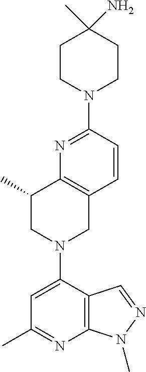 [SMILES: Cc1cc(N2Cc3ccc(N4CCC(C)(N)CC4)nc3[C@@H](C)C2)c2cnn(C)c2n1]

The title compound was prepared in analogy to the preparation of Example 58 by using tert-butyl N-(4-methyl-4-piperidyl)carbamate (CAS: 163271-08-7, Vendor: PharmaBlock) instead of tert-butyl N-(4-piperidyl)carbamate. Example 59 (16 mg) was obtained as a white solid. MS: calc'd 406 (MH⁺), measured 406 (MH⁺). ¹H NMR (400 MHz, METHANOL-d₄) δ=8.18 (s, 1H), 7.42 (d, J=8.7 Hz, 1H), 6.70 (d, J=8.7 Hz, 1H), 6.42-6.36 (m, 1H), 4.76-4.61 (m, 2H), 4.01-3.92 (m, 4H), 3.80-3.67 (m, 3H), 3.55-3.45 (m, 2H), 3.17-3.07 (m, 1H), 2.53 (s, 3H), 1.68-1.55 (m, 4H), 1.39 (d, J=7.0 Hz, 3H), 1.21 (s, 3H).

### Example 60

### N,N-dimethyl-1-[(8S)-6-(1,6-dimethylpyrazolo[3,4-b]pyridin-4-yl)-8-methyl-7,8-dihydro-5H-1,6-naphthyridin-2-yl]piperidin-4-amine

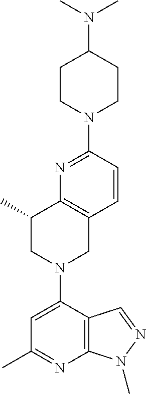 [SMILES: Cc1cc(N2Cc3ccc(N4CCC(N(C)C)CC4)nc3[C@@H](C)C2)c2cnn(C)c2n1]

The title compound was prepared in analogy to the preparation of Example 58 by using N,N-dimethylpiperidin-4-amine (CAS: 50533-97-6, Vendor: Accela) instead of tert-butyl N-(4-piperidyl)carbamate without the Boc deprotection reaction. Example 60 (7 mg) was obtained as a light yellow solid. MS: calc'd 420 (MH⁺), measured 420 (MH⁺). ¹H NMR (400 MHz, METHANOL-d₄) δ=8.16 (s, 1H), 7.40 (d, J=8.6 Hz, 1H), 6.68 (d, J=8.7 Hz, 1H), 6.36 (s, 1H), 4.75-4.60 (m, 2H), 4.49-4.39 (m, 2H), 4.03-3.90 (m, 4H), 3.72 (dd, J=6.8, 12.8 Hz, 1H), 3.10 (br d, J=4.6 Hz, 1H), 2.86-2.71 (m, 2H), 2.52 (s, 3H), 2.40 (br s, 1H), 2.30 (s, 6H), 1.94 (br d, J=12.5 Hz, 2H), 1.53-1.42 (m, 2H), 1.38 (d, J=7.0 Hz, 3H).

### Example 61

### 4-[(dimethylamino)methyl]-1-[(8S)-6-(1,6-dimethylpyrazolo[3,4-b]pyridin-4-yl)-8-methyl-7,8-dihydro-5H-1,6-naphthyridin-2-yl]piperidin-4-ol

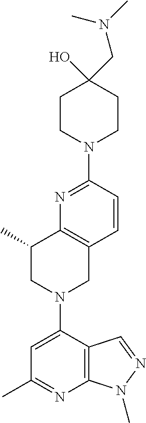 [SMILES: Cc1cc(N2Cc3ccc(N4CCC(O)(CN(C)C)CC4)nc3[C@@H](C)C2)c2cnn(C)c2n1]

The title compound was prepared in analogy to the preparation of Example 58 by using 4-[(dimethylamino)methyl]piperidin-4-ol dihydrochloride (CAS: 695145-47-2, Vendor: J&K Scientific) instead of tert-butyl N-(4-piperidyl)carbamate without the Boc deprotection reaction. Example 61 (15 mg) was obtained as a white solid. MS: calc'd 450 (MH⁺), measured 450 (MH⁺). ¹H NMR (400 MHz, METHANOL-d₄) δ=8.18 (s, 1H), 7.42 (d, J=8.6 Hz, 1H), 6.70 (d, J=8.7 Hz, 1H), 6.38 (s, 1H), 4.76-4.61 (m, 2H), 4.04-3.92 (m, 6H), 3.75 (dd, J=6.7, 12.7 Hz, 1H), 3.37-3.32 (m, 2H), 3.16-3.07 (m, 1H), 2.57-2.51 (m, 3H), 2.38-2.30 (m, 8H), 1.70-1.60 (m, 4H), 1.40 (d, J=7.0 Hz, 3H).

### Example 62

### (8S)-8-methyl-6-(1-methylpyrazolo[3,4-b]pyridin-4-yl)-2-piperazin-1-yl-7,8-dihydro-5H-1,6-naphthyridine

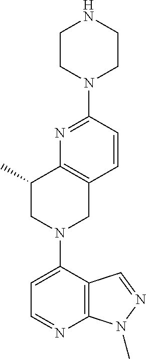 [SMILES: C[C@H]1CN(c2ccnc3c2cnn3C)Cc2ccc(N3CCNCC3)nc21]

The title compound was prepared in analogy to the preparation of Example 58 by using 4-chloro-1-methyl-pyrazolo[3,4-b]pyridine (compound 1a) instead of 4-chloro-1,6-dimethyl-pyrazolo[3,4-b]pyridine and tert-butyl piperazine-1-carboxylate (compound 1c) instead of tert-butyl N-(4-piperidyl)carbamate. Example 62 (16 mg) was obtained as a light yellow solid. MS: calc'd 364 (MH⁺), measured 364 (MH⁺). ¹H NMR (400 MHz, METHANOL-d₄) δ=8.60 (s, 1H), 8.12 (d, J=7.3 Hz, 1H), 7.64 (d, J=8.7 Hz, 1H), 6.92 (d, J=7.5 Hz, 1H), 6.89 (d, J=8.7 Hz, 1H), 5.19-5.02 (m, 2H), 4.22 (dd, J=4.2, 12.9 Hz, 1H), 4.12 (s, 3H), 3.96 (dd, J=7.5, 13.0 Hz, 1H), 3.91-3.83 (m, 4H), 3.36 (br d, J=5.4 Hz, 4H), 3.31-3.26 (m, 1H), 1.46 (d, J=7.0 Hz, 3H).

### Example 63

### (3R,4R)-4-methoxy-1-[(8S)-6-(1,6-dimethylpyrazolo[3,4-b]pyridin-4-yl)-8-methyl-7,8-dihydro-5H-1,6-naphthyridin-2-yl]pyrrolidin-3-amine

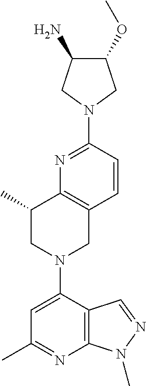 [SMILES: CO[C@@H]1CN(c2ccc3c(n2)[C@@H](C)CN(c2cc(C)nc4c2cnn4C)C3)C[C@H]1N]

The title compound was prepared in analogy to the preparation of Example 58 by using tert-butyl N-[(3R,4R)-4-methoxypyrrolidin-3-yl]carbamate (compound 11b) instead of tert-butyl N-(4-piperidyl)carbamate. Example 63 (50 mg) was obtained as a white solid. MS: calc'd 408 (MH⁺), measured 408 (MH⁺). ¹H NMR (400 MHz, METHANOL-d₄) δ=8.15 (s, 1H), 7.37 (d, J=8.6 Hz, 1H), 6.39-6.32 (m, 2H), 4.70 (d, J=15.0 Hz, 1H), 4.59 (d, J=14.9 Hz, 1H), 3.97 (s, 3H), 3.90-3.73 (m, 4H), 3.67 (dd, J=6.0, 10.5 Hz, 1H), 3.53-3.49 (m, 1H), 3.46 (dd, J=2.6, 10.9 Hz, 1H), 3.42 (s, 3H), 3.30-3.26 (m, 1H), 3.15-3.07 (m, 1H), 2.52 (s, 3H), 1.38 (d, J=7.0 Hz, 3H).

### Example 64

### (8S)-6-(1,6-dimethylpyrazolo[3,4-b]pyridin-4-yl)-8-methyl-2-(4-piperidyloxy)-7,8-dihydro-5H-1,6-naphthyridine

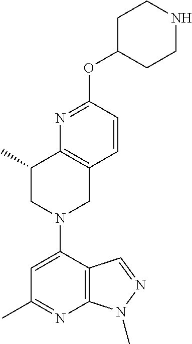 [SMILES: Cc1cc(N2Cc3ccc(OC4CCNCC4)nc3[C@@H](C)C2)c2cnn(C)c2n1]

The title compound was prepared in analogy to the preparation of Example 58 by using tert-butyl 4-hydroxypiperidine-1-carboxylate (CAS: 109384-19-2, Vendor: Accela) instead of tert-butyl N-(4-piperidyl)carbamate. Example 64 (18 mg) was obtained as a white solid. MS: calc'd 393 (MH⁺), measured 393 (MH⁺). ¹H NMR (400 MHz, METHANOL-d₄) δ=8.53 (s, 1H), 7.70 (d, J=8.4 Hz, 1H), 6.83-6.75 (m, 2H), 5.42 (tt, J=3.1, 6.3 Hz, 1H), 5.20-5.08 (m, 1H), 5.07-5.00 (m, 1H), 4.23 (br dd, J=4.1, 13.0 Hz, 1H), 4.11 (s, 3H), 3.94 (dd, J=7.5, 12.9 Hz, 1H), 3.43 (br s, 2H), 3.32-3.23 (m, 3H), 2.69 (s, 3H), 2.32-2.21 (m, 2H), 2.13 (ddd, J=3.3, 7.1, 10.6 Hz, 2H), 1.47 (d, J=7.0 Hz, 3H).

### Example 65

### (3S,4S)-3-methoxy-1-[(8S)-6-(1,6-dimethylpyrazolo[3,4-b]pyridin-4-yl)-8-methyl-7,8-dihydro-5H-1,6-naphthyridin-2-yl]piperidin-4-amine

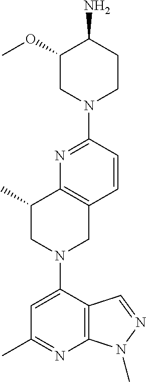 [SMILES: CO[C@H]1CN(c2ccc3c(n2)[C@@H](C)CN(c2cc(C)nc4c2cnn4C)C3)CC[C@@H]1N]

The title compound was prepared in analogy to the preparation of Example 58 by using tert-butyl N-[(3S,4S)-3-methoxypiperidin-4-yl]carbamate hemioxalate (CAS: 2253105-33-6, Vendor: PharmaBlock) instead of tert-butyl N-(4-piperidyl)carbamate. Example 65 (30 mg) was obtained as a white solid. MS: calc'd 422 (MH⁺), measured 422 (MH⁺). ¹H NMR (400 MHz, METHANOL-d₄) δ=8.17 (s, 1H), 7.43 (d, J=8.6 Hz, 1H), 6.72 (d, J=8.6 Hz, 1H), 6.37 (s, 1H), 4.83-4.61 (m, 3H), 4.29-4.18 (m, 1H), 4.02-3.93 (m, 4H), 3.71 (ddd, J=2.6, 7.1, 12.7 Hz, 1H), 3.52 (s, 3H), 3.17-3.07 (m, 1H), 2.99-2.83 (m, 2H), 2.78-2.69 (m, 1H), 2.56-2.46 (m, 4H), 1.95-1.86 (m, 1H), 1.50-1.35 (m, 4H).

### Example 66

### (8S)-6-(1,6-dimethylpyrazolo[3,4-b]pyridin-4-yl)-8-methyl-2-piperazin-1-yl-7,8-dihydro-5H-1,6-naphthyridine

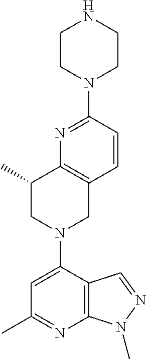 [SMILES: Cc1cc(N2Cc3ccc(N4CCNCC4)nc3[C@@H](C)C2)c2cnn(C)c2n1]

The title compound was prepared in analogy to the preparation of Example 58 by using tert-butyl piperazine-1-carboxylate (compound 1c) instead of tert-butyl N-(4-piperidyl)carbamate. Example 66 (45 mg) was obtained as a white solid. MS: calc'd 378 (MH⁺), measured 378 (MH⁺). ¹H NMR (400 MHz, METHANOL-d₄) δ=8.52 (s, 1H), 7.65 (d, J=8.7 Hz, 1H), 6.91 (d, J=8.7 Hz, 1H), 6.78 (s, 1H), 5.16-5.01 (m, 2H), 4.24-4.14 (m, 1H), 4.11 (s, 3H), 4.02-3.92 (m, 1H), 3.92-3.85 (m, 4H), 3.40-3.34 (m, 4H), 3.32-3.25 (m, 1H), 2.68 (s, 3H), 1.45 (d, J=7.0 Hz, 3H).

### Example 67

### (8S)-6-(1,6-dimethylpyrazolo[3,4-b]pyridin-4-yl)-8-methyl-2-(4-methylpiperazin-1-yl)-7,8-dihydro-5H-1,6-naphthyridine

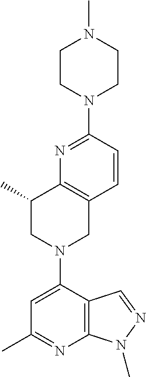 [SMILES: Cc1cc(N2Cc3ccc(N4CCN(C)CC4)nc3[C@@H](C)C2)c2cnn(C)c2n1]

The title compound was prepared in analogy to the preparation of Example 58 by using 1-methylpiperazine (CAS: 109-01-3, Vendor: TCI) instead of tert-butyl N-(4-piperidyl)carbamate without the Boc deprotection reaction. Example 67 (7 mg) was obtained as a yellow solid. MS: calc'd 392 (MH⁺), measured 392 (MH⁺). ¹H NMR (400 MHz, METHANOL-d₄) δ=8.52 (s, 1H), 7.63 (d, J=8.6 Hz, 1H), 6.89 (d, J=8.6 Hz, 1H), 6.77 (s, 1H), 5.08 (br s, 2H), 4.58 (br s, 2H), 4.25-4.17 (m, 1H), 4.11 (s, 3H), 3.97-3.87 (m, 1H), 3.63 (br d, J=1.1 Hz, 2H), 3.32-3.12 (m, 5H), 2.98 (s, 3H), 2.68 (s, 3H), 1.45 (d, J=7.0 Hz, 3H).

### Example 68

### 2-[(8S)-6-(1,6-dimethylpyrazolo[3,4-b]pyridin-4-yl)-8-methyl-7,8-dihydro-5H-1,6-naphthyridin-2-yl]-5-oxa-2,8-diazaspiro[3.5]nonane

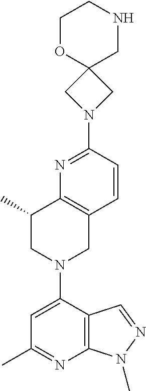 [SMILES: Cc1cc(N2Cc3ccc(N4CC5(CNCCO5)C4)nc3[C@@H](C)C2)c2cnn(C)c2n1]

The title compound was prepared in analogy to the preparation of Example 58 by using tert-butyl 5-oxa-2,8-diazaspiro[3.5]nonane-8-carboxylate (CAS: 1251005-61-4, Vendor: PharmaBlock) instead of tert-butyl N-(4-piperidyl)carbamate. Example 68 (13 mg) was obtained as a white solid. MS: calc'd 420 (MH⁺), measured 420 (MH⁺). ¹H NMR (400 MHz, METHANOL-d₄) δ=8.10 (s, 1H), 7.36 (d, J=8.6 Hz, 1H), 6.29 (s, 1H), 6.26 (d, J=8.4 Hz, 1H), 4.71-4.64 (m, 1H), 4.57-4.50 (m, 1H), 3.92-3.80 (m, 6H), 3.74 (dt, J=3.5, 8.6 Hz, 3H), 3.62-3.55 (m, 2H), 3.09-3.01 (m, 1H), 2.90 (s, 2H), 2.75-2.67 (m, 2H), 2.44 (s, 3H), 1.28 (d, J=7.0 Hz, 3H).

### Example 69

### (3S,4R)-4-fluoro-1-[(8S)-6-(1,6-dimethylpyrazolo[3,4-b]pyridin-4-yl)-8-methyl-7,8-dihydro-5H-1,6-naphthyridin-2-yl]pyrrolidin-3-amine

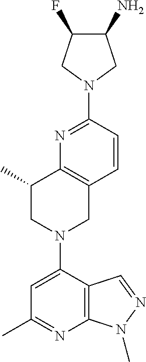 [SMILES: Cc1cc(N2Cc3ccc(N4C[C@@H](F)[C@@H](N)C4)nc3[C@@H](C)C2)c2cnn(C)c2n1]

The title compound was prepared in analogy to the preparation of Example 58 by using tert-butyl N-[(3S,4R)-4-fluoropyrrolidin-3-yl]carbamate (CAS: 1033718-89-6, Vendor: PharmaBlock) instead of tert-butyl N-(4-piperidyl)carbamate. Example 69 (39 mg) was obtained as a white solid. MS: calc'd 396 (MH⁺), measured 396 (MH⁺). ¹H NMR (400 MHz, METHANOL-d₄) δ=8.32 (s, 1H), 7.53 (d, J=8.6 Hz, 1H), 6.54 (s, 1H), 6.48 (d, J=8.6 Hz, 1H), 5.58-5.41 (m, 1H), 4.91 (br s, 1H), 4.82-4.74 (m, 1H), 4.19-4.09 (m, 2H), 4.09-3.99 (m, 4H), 3.95-3.76 (m, 3H), 3.62-3.53 (m, 1H), 3.25-3.17 (m, 1H), 2.60 (s, 3H), 1.44 (d, J=7.0 Hz, 3H).

### Example 70

### 8-[(8S)-6-(1,6-dimethylpyrazolo[3,4-b]pyridin-4-yl)-8-methyl-7,8-dihydro-5H-1,6-naphthyridin-2-yl]-5-oxa-2,8-diazaspiro[3.5]nonane

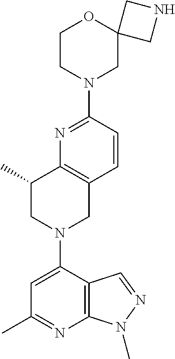 [SMILES: Cc1cc(N2Cc3ccc(N4CCOC5(CNC5)C4)nc3[C@@H](C)C2)c2cnn(C)c2n1]

The title compound was prepared in analogy to the preparation of Example 58 by using tert-butyl 5-oxa-2,8-diazaspiro[3.5]nonane-2-carboxylate (CAS: 1251011-05-8, Vendor: PharmaBlock) instead of tert-butyl N-(4-piperidyl)carbamate. Example 70 (14 mg) was obtained as a light yellow solid. MS: calc'd 420 (MH⁺), measured 420 (MH⁺). ¹H NMR (400 MHz, METHANOL-d₄) δ=8.40 (s, 1H), 7.50 (d, J=8.7 Hz, 1H), 6.74 (d, J=8.6 Hz, 1H), 6.66 (s, 1H), 5.04-4.94 (m, 1H), 4.88 (br d, J=16.5 Hz, 1H), 3.99 (s, 8H), 3.86 (br dd, J=7.1, 13.0 Hz, 1H), 3.79-3.73 (m, 3H), 3.72-3.67 (m, 1H), 3.44-3.37 (m, 2H), 3.19-3.14 (m, 1H), 2.56 (s, 3H), 1.35 (d, J=7.0 Hz, 3H).

### Example 72

### (8R)-6-(1,6-dimethylpyrazolo[3,4-b]pyridin-4-yl)-8-methyl-2-piperazin-1-yl-7,8-dihydro-5H-1,6-naphthyridine

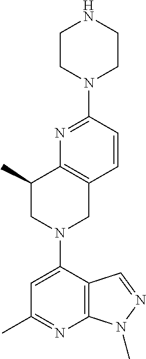 [SMILES: Cc1cc(N2Cc3ccc(N4CCNCC4)nc3[C@H](C)C2)c2cnn(C)c2n1]

The title compound was prepared in analogy to the preparation of Example 58 by using Intermediate D instead of Intermediate C and tert-butyl piperazine-1-carboxylate (compound 1c) instead of tert-butyl N-(4-piperidyl)carbamate. Example 72 (8 mg) was obtained as a light yellow solid. MS: calc'd 378 (MH⁺), measured 378 (MH⁺). ¹H NMR (400 MHz, METHANOL-d₄) δ=8.21 (s, 1H), 7.48 (d, J=8.6 Hz, 1H), 6.71 (d, J=8.6 Hz, 1H), 6.41 (s, 1H), 4.77-4.65 (m, 2H), 4.05-3.96 (m, 4H), 3.78 (dd, J=6.7, 12.8 Hz, 1H), 3.59-3.48 (m, 4H), 3.20-3.10 (m, 1H), 3.01-2.91 (m, 4H), 2.56 (s, 3H), 1.42 (d, J=7.0 Hz, 3H).

### Example 73

### (3S,4S)-3-methoxy-1-[(5R)-7-(1,6-dimethylpyrazolo[3,4-b]pyridin-4-yl)-5-methyl-6,8-dihydro-5H-2,7-naphthyridin-3-yl]piperidin-4-amine

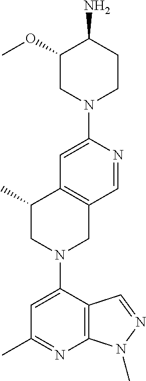 [SMILES: CO[C@H]1CN(c2cc3c(cn2)CN(c2cc(C)nc4c2cnn4C)C[C@@H]3C)CC[C@@H]1N]

The title compound was prepared in analogy to the preparation of Example 1 by using 4-chloro-1,6-dimethyl-pyrazolo[3,4-b]pyridine (CAS: 19867-78-8, Vendor: PharmaBlock) instead of compound 1a, Intermediate E instead of Intermediate A, tert-butyl N-[(3S,4S)-3-methoxypiperidin-4-yl]carbamate hemioxalate (CAS: 2253105-33-6, Vendor: PharmaBlock) instead of compound 1c. Example 73 (23 mg) was obtained as a yellow solid. MS: calc'd 422 (MH⁺), measured 422 (MH⁺).

¹H NMR (400 MHz, METHANOL-d₄) δ=8.52 (s, 1H), 8.18 (s, 1H), 7.28 (s, 1H), 6.74 (s, 1H), 5.20 (br d, J=15.4 Hz, 1H), 4.82-4.72 (m, 1H), 4.42-4.31 (m, 1H), 4.13 (s, 4H), 3.84 (br dd, J=8.0, 12.3 Hz, 1H), 3.60 (s, 3H), 3.53-3.40 (m, 2H), 3.38-3.32 (m, 2H), 3.31-3.20 (m, 1H), 2.96 (dd, J=10.3, 13.1 Hz, 1H), 2.70 (s, 3H), 2.31-2.20 (m, 1H), 1.84 (dq, J=4.4, 12.6 Hz, 1H), 1.52 (d, J=6.9 Hz, 3H).

### Example 74

### (4R)-2-(1,6-dimethylpyrazolo[3,4-b]pyridin-4-yl)-4-methyl-6-piperazin-1-yl-3,4-dihydro-1H-2,7-naphthyridine

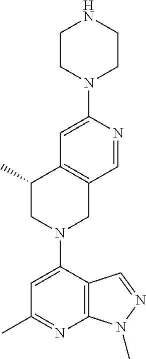 [SMILES: Cc1cc(N2Cc3cnc(N4CCNCC4)cc3[C@@H](C)C2)c2cnn(C)c2n1]

The title compound was prepared in analogy to the preparation of Example 73 by using tert-butyl piperazine-1-carboxylate (compound 1c) instead of tert-butyl N-[(3S,4S)-3-methoxypiperidin-4-yl]carbamate hemioxalate. Example 74 (19 mg) was obtained as a yellow solid. MS: calc'd 378 (MH⁺), measured 378 (MH⁺). ¹H NMR (400 MHz, METHANOL-d₄) δ=8.50 (s, 1H), 8.19 (s, 1H), 6.97 (s, 1H), 6.72 (s, 1H), 5.12 (br d, J=15.2 Hz, 1H), 4.95-4.90 (m, 1H), 4.12-4.04 (m, 4H), 3.88-3.76 (m, 5H), 3.38-3.30 (m, 5H), 2.67 (s, 3H), 1.46 (d, J=6.8 Hz, 3H).

### Example 75

### (4R)-6-(4,7-diazaspiro[2.5]octan-7-yl)-2-(1,6-dimethylpyrazolo[3,4-b]pyridin-4-yl)-4-methyl-3,4-dihydro-1H-2,7-naphthyridine

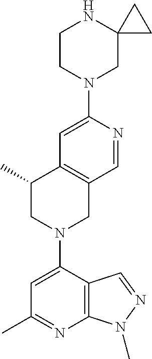 [SMILES: Cc1cc(N2Cc3cnc(N4CCNC5(CC5)C4)cc3[C@@H](C)C2)c2cnn(C)c2n1]

The title compound was prepared in analogy to the preparation of Example 73 by using tert-butyl 4,7-diazaspiro[2.5]octane-4-carboxylate (CAS: 674792-08-6, Vendor: Accela) instead of tert-butyl N-[(3S,4S)-3-methoxypiperidin-4-yl]carbamate hemioxalate. Example 75 (1.2 mg) was obtained as a white solid. MS: calc'd 404 (MH⁺), measured 404 (MH⁺). ¹H NMR (400 MHz, METHANOL-d₄) δ=8.22 (s, 1H), 8.06 (s, 1H), 6.74 (s, 1H), 6.38 (s, 1H), 4.81 (s, 1H), 4.73-4.67 (m, 1H), 4.01 (s, 3H), 3.88 (dd, J=4.3, 12.2 Hz, 1H), 3.75-3.64 (m, 1H), 3.58-3.51 (m, 2H), 3.41 (s, 2H), 3.26-3.16 (m, 1H), 3.06-3.00 (m, 2H), 2.56 (s, 3H), 1.43 (d, J=7.0 Hz, 3H), 0.66 (s, 4H).

### Example 76

### (4R)-2-(1,6-dimethylpyrazolo[3,4-b]pyridin-4-yl)-4-methyl-6-[[(7R,8aS)-1,2,3,4,6,7,8,8a-octahydropyrrolo[1,2-a]pyrazin-7-yl]oxy]-3,4-dihydro-1H-2,7-naphthyridine

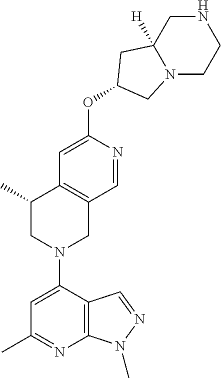 [SMILES: Cc1cc(N2Cc3cnc(O[C@@H]4C[C@H]5CNCCN5C4)cc3[C@@H](C)C2)c2cnn(C)c2n1]

The title compound was prepared in analogy to the preparation of Example 58 by using Intermediate E instead of Intermediate C and tert-butyl (7R,8aS)-7-hydroxy-octahydropyrrolo[1,2-a]piperazine-2-carboxylate (CAS: 1204603-42-8, Vendor: PharmaBlock) instead of tert-butyl N-(4-piperidyl)carbamate. Example 76 (14.5 mg) was obtained as a light yellow solid. MS: calc'd 434 (MH⁺), measured 434 (MH⁺). ¹H NMR (400 MHz, METHANOL-d₄) δ=8.39 (br s, 1H), 8.06 (s, 1H), 6.74 (s, 1H), 6.62 (s, 1H), 5.55-5.38 (m, 1H), 5.04 (br d, J=15.0 Hz, 1H), 4.00 (s, 4H), 3.94 (dd, J=6.5, 11.6 Hz, 1H), 3.65-3.54 (m, 2H), 3.44 (td, J=2.7, 13.2 Hz, 2H), 3.37-3.21 (m, 4H), 3.12-2.97 (m, 2H), 2.91 (br dd, J=3.8, 11.6 Hz, 1H), 2.57 (s, 3H), 2.29-2.19 (m, 1H), 2.18-2.06 (m, 1H), 1.35 (d, J=6.8 Hz, 3H).

### Example 77

### (4R)-2-(1,6-dimethylpyrazolo[3,4-b]pyridin-4-yl)-4-methyl-6-(2-piperazin-1-ylethoxy)-3,4-dihydro-1H-2,7-naphthyridine

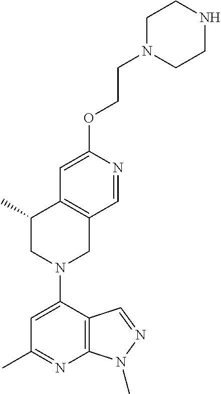 [SMILES: Cc1cc(N2Cc3cnc(OCCN4CCNCC4)cc3[C@@H](C)C2)c2cnn(C)c2n1]

The title compound was prepared in analogy to the preparation of Example 73 by using tert-butyl 4-(2-hydroxyethyl)piperazine-1-carboxylate (CAS: 77279-24-4, Vendor: Bepharm) instead of tert-butyl N-[(3S,4S)-3-methoxypiperidin-4-yl]carbamate hemioxalate. Example 77 (45.9 mg) was obtained as a light yellow solid. MS: calc'd 422 (MH⁺), measured 422 (MH⁺). ¹H NMR (400 MHz, METHANOL-d₄) δ=8.51 (br s, 1H), 8.19 (s, 1H), 6.91 (s, 1H), 6.74 (s, 1H), 5.17 (br d, J=14.8 Hz, 1H), 4.88-4.57 (m, 3H), 4.12 (s, 4H), 3.74-3.56 (m, 11H), 3.41-3.33 (m, 1H), 2.69 (s, 3H), 1.48 (d, J=6.9 Hz, 3H).

### Example 78

### (3R,4R)-4-methoxy-1-[(5R)-7-(1,6-dimethylpyrazolo[3,4-b]pyridin-4-yl)-5-methyl-6,8-dihydro-5H-2,7-naphthyridin-3-yl]pyrrolidin-3-amine

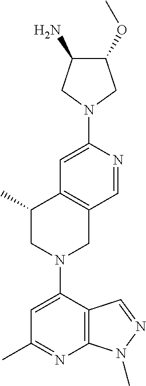 [SMILES: CO[C@@H]1CN(c2cc3c(cn2)CN(c2cc(C)nc4c2cnn4C)C[C@@H]3C)C[C@H]1N]

The title compound was prepared in analogy to the preparation of Example 73 by using tert-butyl N-[(3R,4R)-4-methoxypyrrolidin-3-yl]carbamate (compound 11b) instead of tert-butyl N-[(3S,4S)-3-methoxypiperidin-4-yl]carbamate hemioxalate. Example 78 (10.0 mg) was obtained as a yellow solid. MS: calc'd 408 (MH⁺), measured 408 (MH⁺). ¹H NMR (400 MHz, METHANOL-d₄) δ=8.51 (s, 1H), 8.13 (s, 1H), 6.99 (s, 1H), 6.73 (s, 1H), 5.21 (br d, J=15.2 Hz, 1H), 4.98 (br s, 1H), 4.31 (br d, J=2.4 Hz, 1H), 4.20-4.03 (m, 7H), 3.89 (br d, J=9.7 Hz, 1H), 3.83-3.73 (m, 2H), 3.55-3.46 (m, 4H), 2.75-2.64 (m, 3H), 1.54 (d, J=6.7 Hz, 3H).

### Example 79

### 2-[(5R)-7-(1,6-dimethylpyrazolo[3,4-b]pyridin-4-yl)-5-methyl-6,8-dihydro-5H-2,7-naphthyridin-3-yl]-5-oxa-2,8-diazaspiro[3.5]nonane

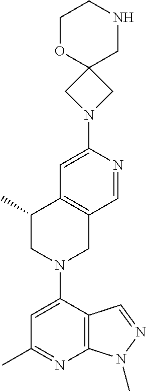 [SMILES: Cc1cc(N2Cc3cnc(N4CC5(CNCCO5)C4)cc3[C@@H](C)C2)c2cnn(C)c2n1]

The title compound was prepared in analogy to the preparation of Example 73 by using tert-butyl 5-oxa-2,8-diazaspiro[3.5]nonane-8-carboxylate (CAS: 1251005-61-4, Vendor: PharmaBlock) instead of tert-butyl N-[(3S,4S)-3-methoxypiperidin-4-yl]carbamate hemioxalate. Example 79 (4.0 mg) was obtained as a white solid. MS: calc'd 420 (MH⁺), measured 420 (MH⁺). ¹H NMR (400 MHz, METHANOL-d₄) δ=8.21 (s, 1H), 8.01 (s, 1H), 6.43 (s, 1H), 6.37 (s, 1H), 4.80 (s, 1H), 4.74-4.66 (m, 1H), 4.03-3.98 (m, 5H), 3.91 (dd, J=4.3, 12.0 Hz, 1H), 3.85 (d, J=8.8 Hz, 2H), 3.75-3.68 (m, 2H), 3.64 (dd, J=7.5, 12.1 Hz, 1H), 3.26-3.17 (m, 1H), 3.02 (s, 2H), 2.86-2.78 (m, 2H), 2.56 (s, 3H), 1.44 (d, J=6.9 Hz, 3H).

### Example 80

### (3R,4R)-3-fluoro-1-[(5R)-7-(1,6-dimethylpyrazolo[3,4-b]pyridin-4-yl)-5-methyl-6,8-dihydro-5H-2,7-naphthyridin-3-yl]piperidin-4-amine

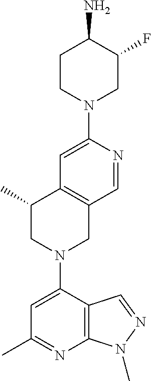 [SMILES: Cc1cc(N2Cc3cnc(N4CC[C@@H](N)[C@H](F)C4)cc3[C@@H](C)C2)c2cnn(C)c2n1]

The title compound was prepared in analogy to the preparation of Example 73 by using tert-butyl N-[(3R,4R)-3-fluoro-4-piperidyl]carbamate (CAS: 1523530-29-1, Vendor: PharmaBlock) instead of tert-butyl N-[(3S,4S)-3-methoxypiperidin-4-yl]carbamate hemioxalate. Example 80 (22.3 mg) was obtained as a light yellow solid. MS: calc'd 410 (MH⁺), measured 410 (MH⁺). ¹H NMR (400 MHz, METHANOL-d₄) δ=8.50 (s, 1H), 8.17 (s, 1H), 7.11 (s, 1H), 6.73 (s, 1H), 5.15 (br d, J=15.2 Hz, 1H), 4.83-4.72 (m, 2H), 4.64 (dt, J=5.2, 9.8 Hz, 1H), 4.37 (br d, J=12.4 Hz, 1H), 4.11 (s, 4H), 3.81 (br dd, J=7.9, 12.3 Hz, 1H), 3.66-3.56 (m, 1H), 3.45-3.35 (m, 1H), 3.23-3.02 (m, 2H), 2.69 (s, 3H), 2.35-2.19 (m, 1H), 1.82 (dq, J=4.4, 12.6 Hz, 1H), 1.49 (d, J=6.9 Hz, 3H).

### Example 81

### (4R)-2-(1,6-dimethylpyrazolo[3,4-b]pyridin-4-yl)-4-methyl-6-[(3R)-3-(methoxymethyl)piperazin-1-yl]-3,4-dihydro-1H-2,7-naphthyridine

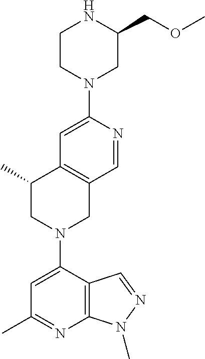

The title compound was prepared in analogy to the preparation of Example 73 by using tert-butyl (2R)-2-(methoxymethyl)piperazine-1-carboxylate (CAS: 1023301-73-6, Vendor: PharmaBlock) instead of tert-butyl N-[(3S,4S)-3-methoxypiperidin-4-yl]carbamate hemioxalate. Example 81 (11.1 mg) was obtained as a yellow solid. MS: calc'd 422 (MH⁺), measured 422 (MH⁺). ¹H NMR (400 MHz, METHANOL-d₄) δ=8.51 (s, 1H), 8.20 (s, 1H), 7.00 (s, 1H), 6.83-6.71 (m, 1H), 5.13 (br d, J=15.2 Hz, 1H), 4.99-4.95 (m, 1H), 4.50-4.37 (m, 2H), 4.18-4.04 (m, 4H), 3.84-3.72 (m, 2H), 3.69-3.58 (m, 2H), 3.51 (br s, 4H), 3.40-3.35 (m, 1H), 2.74-2.65 (m, 3H), 3.32-3.17 (m, 3H), 1.48 (d, J=6.9 Hz, 3H).

### Example 82

The following tests were carried out in order to determine the activity of the compounds of formula (I) and (Ia) in HEK293-Blue-hTLR-7/8/9 cells assay.

### HEK293-Blue-hTLR-7 Cells Assay:

A stable HEK293-Blue-hTLR-7 cell line was purchased from InvivoGen (Cat. #: hkb-htlr7, San Diego, California, USA). These cells were originally designed for studying the stimulation of human TLR7 by monitoring the activation of NF-κB. A SEAP (secreted embryonic alkaline phosphatase) reporter gene was placed under the control of the IFN-β minimal promoter fused to five NF-κB and AP-1-binding sites. The SEAP was induced by activating NF-κB and AP-1 via stimulating HEK-Blue hTLR7 cells with TLR7 ligands. Therefore the reporter expression w % as declined by TLR7 antagonist under the stimulation of a ligand, such as R848 (Resiquimod), for incubation of 20 hrs. The cell culture supernatant SEAP reporter activity was determined using QUANTI-Blue™ kit (Cat. #: rep-qb1, Invivogen, San Diego, Ca, USA) at a wavelength of 640 nm, a detection medium that turns purple or blue in the presence of alkaline phosphatase.

HEK293-Blue-hTLR7 cells were incubated at a density of 250,000˜450,000 cells/mL in a volume of 170 μL in a 96-well plate in Dulbecco's Modified Eagle's medium (DMEM) containing 4.5 g/L glucose, 50 U/mL penicillin, 50 mg/mL streptomycin, 100 mg/nL Normocin, 2 mM L-glutamine, 10% (v/v) heat-inactivated fetal bovine serum with addition of 20 μL test compound in a serial dilution in the presence of final DMSO at 1% and 10 μL of 20 uM R848 in above DMEM, perform incubation under 37° C. in a C02 incubator for 20 hrs. Then 20 μL of the supernatant from each well was incubated with 180 μL Quanti-blue substrate solution at 37° C. for 2 hrs and the absorbance was read at 620˜655 nm using a spectrophotometer. The signaling pathway that TLR7 activation leads to downstream NF-κB activation has been widely accepted, and therefore similar reporter assay was modified for evaluating TLR7 antagonist.

### HEK293-Blue-hTLR-8 Cells Assay:

A stable HEK293-Blue-hTLR-8 cell line was purchased from InvivoGen (Cat. #: hkb-htlr8, San Diego, California, USA). These cells were originally designed for studying the stimulation of human TLR8 by monitoring the activation of NF-κB. A SEAP (secreted embryonic alkaline phosphatase) reporter gene was placed under the control of the IFN-β minimal promoter fused to five NF-κB and AP-1-binding sites. The SEAP was induced by activating NF-κB and AP-1 via stimulating HEK-Blue hTLR8 cells with TLR8 ligands. Therefore the reporter expression was declined by TLR8 antagonist under the stimulation of a ligand, such as R848, for incubation of 20 hrs. The cell culture supernatant SEAP reporter activity was determined using QUANTI-Blue™ kit (Cat. #: rep-qb1, Invivogen, San Diego, Ca, USA) at a wavelength of 640 nm, a detection medium that turns purple or blue in the presence of alkaline phosphatase.

HEK293-Blue-hTLR8 cells were incubated at a density of 250,000˜450,000 cells/mL in a volume of 170 μL in a 96-well plate in Dulbecco's Modified Eagle's medium (DMEM) containing 4.5 g/L glucose, 50 U/nL penicillin, 50 mg/mL streptomycin, 100 mg/nL Normocin, 2 mM L-glutamine, 10% (v/v) heat-inactivated fetal bovine serum with addition of 20 μL test compound in a serial dilution in the presence of final DMSO at 1% and 10 μL of 60 uM R848 in above DMEM, perform incubation under 37° C. in a C02 incubator for 20 hrs. Then 20 μL of the supernatant from each well was incubated with 180 μL Quanti-blue substrate solution at 37° C. for 2 hrs and the absorbance was read at 620-655 nm using a spectrophotometer. The signaling pathway that TLR8 activation leads to downstream NF-κB activation has been widely accepted, and therefore similar reporter assay was modified for evaluating TLR8 antagonist.

### HEK293-Blue-hTLR-9 Cells Assay:

A stable HEK293-Blue-hTLR-9 cell line was purchased from InvivoGen (Cat. #: hkb-htlr9, San Diego, California, USA). These cells were originally designed for studying the stimulation of human TLR9 by monitoring the activation of NF-κB. A SEAP (secreted embryonic alkaline phosphatase) reporter gene was placed under the control of the IFN-β minimal promoter fused to five NF-κB and AP-1-binding sites. The SEAP was induced by activating NF-κB and AP-1 via stimulating HEK-Blue hTLR9 cells with TLR9 ligands. Therefore the reporter expression was declined by TLR9 antagonist under the stimulation of a ligand, such as ODN2006 (Cat. #: tlrl-2006-1, Invivogen, San Diego, California, USA), for incubation of 20 hrs. The cell culture supernatant SEAP reporter activity was determined using QUANTI-Blue™ kit (Cat. #: rep-qb1, Invivogen, San Diego, California, USA) at a wavelength of 640 nm, a detection medium that turns purple or blue in the presence of alkaline phosphatase.

HEK293-Blue-hTLR9 cells were incubated at a density of 250,000-450,000 cells/mL in a volume of 170 μL in a 96-well plate in Dulbecco's Modified Eagle's medium (DMEM) containing 4.5 g/L glucose, 50 U/mL penicillin, 50 mg/mL streptomycin, 100 mg/ml Normocin, 2 mM L-glutamine, 10% (v/v) heat-inactivated fetal bovine serum with addition of 20 μL test compound in a serial dilution in the presence of final DMSO at 1% and 10 μL of 20 uM ODN2006 in above DMEM, perform incubation under 37° C. in a CO₂ incubator for 20 hrs. Then 20 μL of the supernatant from each well was incubated with 180 μL Quanti-blue substrate solution at 37° C. for 2 h and the absorbance was read at 620˜655 nm using a spectrophotometer.

The signaling pathway that TLR9 activation leads to downstream NF-κB activation has been widely accepted, and therefore similar reporter assay was modified for evaluating TLR9 antagonist.

The compounds of formula (I) or (Ia) have human TLR7 and/or TLR8 inhibitory activities (IC₅₀ value)<0.5 μM. Moreover, some compounds also have human TLR9 inhibitory activity <0.5 μM. Activity data of the compounds of the present invention were shown in Table 2.

TABLE 2
The activity of the compounds of present invention
in HEK293-Blue-hTLR-7/8/9 cells assays

| | HEK/hTLR7 | HEK/hTLR8 | HEK/hTLR9 |
|---|---|---|---|
| Example No | IC₅₀ (μM) | IC₅₀ (μM) | IC₅₀ (μM) |
| 1 | 0.003 | 0.002 | 0.198 |
| 2 | 0.008 | 0.001 | 0.086 |
| 3 | 0.019 | 0.056 | 0.185 |
| 4 | 0.004 | 0.007 | 0.275 |
| 5 | 0.003 | 0.010 | 0.123 |
| 6 | 0.026 | 0.018 | 0.112 |
| 7 | 0.018 | 0.017 | 0.104 |
| 8 | 0.006 | 0.006 | 0.140 |
| 9 | 0.007 | 0.011 | 0.117 |
| 10 | 0.001 | 0.001 | 0.058 |
| 11 | 0.002 | <0.0003 | 0.063 |
| 12 | 0.009 | 0.008 | 0.298 |
| 13 | 0.007 | 0.002 | 0.399 |
| 14 | 0.008 | 0.001 | 0.158 |
| 15 | 0.004 | 0.003 | 0.309 |
| 16 | 0.005 | 0.004 | 0.063 |
| 17 | 0.008 | 0.011 | 0.217 |
| 18 | 0.008 | 0.004 | 0.111 |
| 19 | 0.008 | 0.004 | 0.101 |
| 20 | 0.003 | 0.017 | 0.239 |
| 21 | 0.007 | 0.014 | 0.372 |
| 22 | 0.010 | 0.016 | 0.412 |
| 23 | 0.005 | 0.007 | 0.115 |
| 24 | 0.010 | 0.002 | 0.311 |
| 25 | 0.015 | <0.0003 | 0.291 |
| 26 | 0.007 | 0.010 | 0.073 |
| 27 | 0.004 | 0.003 | 0.185 |
| 28 | 0.004 | <0.0003 | 0.150 |
| 29 | 0.009 | 0.002 | 0.106 |
| 30 | 0.037 | 0.002 | 0.088 |
| 31 | 0.024 | 0.002 | 0.105 |
| 33 | 0.006 | 0.015 | 0.326 |
| 34 | 0.026 | 0.026 | 0.198 |
| 35 | 0.024 | 0.014 | 0.182 |
| 37 | 0.014 | 0.005 | 0.222 |
| 38 | 0.003 | <0.0003 | 0.147 |
| 40 | 0.008 | 0.007 | 0.326 |
| 41 | 0.007 | 0.008 | 0.047 |
| 42 | 0.003 | 0.002 | 0.398 |
| 43 | 0.008 | 0.002 | 0.242 |
| 44 | 0.015 | 0.005 | 0.234 |
| 45 | 0.012 | 0.010 | 0.350 |
| 46 | 0.010 | 0.007 | 0.360 |
| 47 | 0.013 | 0.003 | 0.230 |
| 48 | 0.013 | 0.006 | 0.229 |
| 49 | 0.005 | 0.002 | 0.199 |
| 50 | 0.010 | 0.003 | 0.376 |
| 51 | 0.009 | 0.004 | 0.365 |
| 52 | 0.009 | 0.003 | 0.117 |
| 53 | 0.018 | 0.006 | 0.210 |
| 54 | 0.053 | 0.005 | 0.334 |
| 55 | 0.007 | 0.006 | 0.137 |
| 56 | 0.009 | 0.007 | 0.254 |
| 57 | 0.008 | 0.024 | 0.226 |
| 58 | 0.002 | 0.0004 | 0.199 |
| 59 | 0.002 | 0.0005 | 0.121 |
| 60 | 0.003 | 0.0005 | 0.104 |
| 61 | 0.003 | 0.0005 | 0.129 |
| 62 | 0.005 | 0.002 | 0.264 |
| 63 | 0.005 | 0.016 | 0.236 |
| 64 | 0.005 | 0.004 | 0.249 |
| 65 | 0.006 | 0.001 | 0.106 |
| 66 | 0.008 | 0.001 | 0.132 |
| 67 | 0.011 | 0.001 | 0.282 |
| 68 | 0.017 | 0.017 | 0.332 |
| 69 | 0.030 | 0.009 | 0.488 |
| 70 | 0.036 | 0.028 | 0.300 |
| 72 | 0.021 | 0.002 | 0.113 |
| 73 | 0.004 | 0.002 | 0.115 |
| 74 | 0.006 | 0.002 | 0.165 |
| 75 | 0.010 | 0.013 | 0.115 |
| 76 | 0.019 | 0.016 | 0.144 |
| 77 | 0.019 | 0.016 | 0.144 |
| 78 | 0.045 | 0.052 | 0.335 |
| 79 | 0.019 | 0.023 | 0.131 |
| 80 | 0.012 | 0.034 | 0.073 |
| 81 | 0.041 | 0.008 | 0.218 |

### Example 83

hERG channel inhibition assay: The hERG channel inhibition assay is a highly sensitive measurement that identifies compounds exhibiting hERG inhibition related to cardiotoxicity in vivo. The hERG K⁺ channels were cloned in humans and stably expressed in a CHO (Chinese hamster ovary) cell line. CHO_{hERG} cells were used for patch-clamp (voltage-clamp, whole-cell) experiments. Cells were stimulated by a voltage pattern to activate hERG channels and conduct I_{KhERG} currents (rapid delayed outward rectifier potassium current of the hERG channel). After the cells were stabilized for a few minutes, the amplitude and kinetics of I_{KhERG} were recorded at a stimulation frequency of 0.1 Hz (6 bpm). Thereafter, the test compound was added to the preparation at increasing concentrations. For each concentration, an attempt was made to reach a steady-state effect, usually, this was achieved within 3-10 min at which time the next highest concentration was applied. The amplitude and kinetics of I_{KhERG} are recorded in each concentration of the drug which were compared to the control values (taken as 100%). (references: Redfern W S, Carlsson L, Davis A S, Lynch W G, MacKenzie I, Palethorpe S, Siegl P K, Strang I, Sullivan A T, Wallis R, Camm A J, Hammond T G. 2003; Relationships between preclinical cardiac electrophysiology, clinical QT interval prolongation and torsade de pointes for a broad range of drugs: evidence for a provisional safety margin in drug development. Cardiovasc. Res. 58:32-45, Sanguinetti MC, Tristani-Firouzi M. 2006; hERG potassium channels and cardiac arrhythmia. Nature 440:463-469, Webster R, Leishman D, Walker D. 2002; Towards a drug concentration effect relationship for QT prolongation and torsades de pointes. Curr. Opin. Drug Discov. Devel. 5:116-26).

Results of hERG are given in Table 3. A safety ratio (hERG IC₂₀/EC₅₀)>30 suggests a sufficient window to differentiate the pharmacology by inhibiting TLR7/8/9 pathways from the potential hERG related cardiotoxicity. According to the calculation of hERG IC₂₀/TLR7/8/9 IC₅₀ below which serves as early selectivity index to assess hERG liability, obviously reference compounds ER-887258, ER-888285, ER-888286, R1 and R² have much narrower safety window compared to the compounds of this invention.

TABLE 3
hERG and safety ratio results

| | | | hERG | hERG | hERG |
|---|---|---|---|---|---|
| | hERG | hERG | IC₂₀/ | IC₂₀/ | IC₂₀/ |
| Example | IC₂₀ | IC₅₀ | TLR7 | TLR8 | TLR9 |
| No | (μM) | (μM) | IC₅₀ | IC₅₀ | IC₅₀ |
| 1 | 6.4 | >20.0 | 2133.3 | 3200.0 | 32.3 |
| 3 | >10 | >20.0 | >526.3 | >178.6 | >54.1 |
| 16 | >10 | >20.0 | >2000.0 | >2500.0 | >158.7 |
| 18 | >10 | >20.0 | >1250.0 | >2500.0 | >90.1 |
| 19 | 6.3 | >20.0 | 787.5 | 1575.0 | 62.4 |
| 20 | 6.2 | >20.0 | 2066.7 | 364.7 | 25.9 |
| 21 | >10 | >20.0 | >1428.6 | >714.3 | >26.9 |
| 25 | >10 | >20.0 | >666.7 | >33333.3 | >34.4 |
| 30 | 6.5 | >20.0 | 175.7 | 3250.0 | 73.9 |
| 31 | 8.5 | >20.0 | 354.2 | 4250.0 | 81.0 |
| 34 | >10 | >20.0 | >384.6 | >384.6 | >50.5 |
| 40 | >10 | >20.0 | >1250.0 | >1428.6 | >30.7 |
| 41 | 5.1 | >20.0 | 728.6 | 637.5 | 108.5 |
| 42 | >10 | >20.0 | >3333.3 | >5000.0 | >25.1 |
| 44 | >10 | >20.0 | >666.7 | >2000.0 | >42.7 |
| 53 | >10 | >20.0 | >555.6 | >1666.7 | >47.6 |
| 73 | 6.9 | >20.0 | 1725.0 | 3450.0 | 60.0 |
| 76 | >10.0 | >20.0 | >526.3 | >625.0 | >69.4 |
| 77 | >10.0 | >20.0 | >526.3 | >625.0 | >69.4 |
| 80 | >10.0 | >20.0 | >833.3 | >294.1 | >137.0 |

### Example 84

### Human PBMC Cell-Based Assay

Unlike the HEK reporter cell lines, human peripheral blood mononuclear cell (PBMC) represents primary human immune cells in blood mainly consisting of lymphocytes, monocytes, and dendritic cells. These cells express TLR7, TLR8, or TLR9, and therefore are natural responders to respective ligand stimulation. Upon activation of these TLRs, PBMCs secrete similar cytokines and chemokines in vitro and in vivo, and therefore the in vitro potency of a TLR7/8/9 antagonist in human PBMC is readily translatable to its pharmacodynamics response in vivo.

Human peripheral blood mononuclear cells (PBMC) were isolated from freshly-drawn lithium-heparinized (Lithium Heparin Plus blood Collection tube, BD Vacutainer®) healthy donor whole blood by density gradient (Ficoll-Paque™ PLUS, GE Healthcare life Sciences). Briefly, 50 mL of blood was diluted with 25 mL PBS (without Ca²⁺, Mg²⁺) in a 50 mL conical tube with porous barrier (Leucosep tube, Greiner bio-one), where 15.5 mL Ficoll-Paque was under laid after spinning. Tubes were centrifuged for 20 minutes at 800×g (1946 rpm) with the brake in the off position, and PBMC were collected from the buffy coat. Cells were then washed twice in PBS, and red blood cells were lysed by suspension in 2 mL (Red Blood Cell Lysis Buffer, Alfa Aesar) for 5-10 minutes at room temperature. After a final wash in PBS, PBMC were resuspended at a final concentration of 2×10⁶ cells/mL in RPMI-1640 media with GlutaMAX™ (Gibco) supplemented with 10% Fetal Bovine Serum (Sigma) and plated at 150 μL/well (3×10⁵ cells/well) in tissue culture treated round bottom 96-well plates (Corning Incorporated). Antagonist compounds (compounds of this invention) solubilized and serial diluted in 100% DMSO were added in duplicate to cells to yield a final concentration of 1% DMSO (v/v). PBMC were incubated with antagonist compounds for 30 minutes at 37° C., 5% CO₂ before adding various TLR agonist reagents in 48 μL complete media per well as follows (final concentrations indicated): CpG ODN 2216 (InvivoGen) at 1 μM for TLR9, ORN 06/LyoVec (InvivoGen) at 1p g/mL for TLR8 and R848 (InvivoGen) at 1p g/mL for TLR7 and TLR8. PBMC were incubated overnight at 37° C. with 5% CO₂. Cell culture supernatants were collected, and levels of various human cytokines were assessed by Luminex assay (ProcartaPlex™ Multiplex Immunoassay, Invitrogen) or ELISA procedure according to the manufacturer's recommended protocol (eBioscience, ThermoFisher Scientific). Viability of the cells was also checked with Cell Viability Assay (CellTiter Glo®Luminescent Cell Viability Assay, Promega).

TABLE 4
hPBMC results

| | hPBMC/TLR9 |
|---|---|
| Example No | IC₅₀ (μM) |
| 1 | 0.396 |
| 3 | 0.434 |
| 4 | 0.265 |
| 5 | 0.156 |
| 9 | 0.326 |
| 18 | 0.365 |
| 19 | 0.395 |
| 23 | 0.240 |
| 28 | 0.271 |
| 31 | 0.214 |
| 41 | 0.286 |
| 47 | 0.290 |
| 50 | 0.475 |
| 58 | 0.277 |
| 63 | 0.430 |
| 65 | 0.371 |
| 73 | 0.340 |
| 74 | 0.250 |

### Example 85

### Human Microsome Stability Assay

The human microsomal stability assay is used for early assessment of metabolic stability of a test compound in human liver microsomes.

Human liver microsomes (Cat. NO.: 452117, Corning, USA; Cat. NO.: H2610, Xenotech, USA) were preincubated with test compound for 10 minutes at 37° C. in 100 mM potassium phosphate buffer, pH 7.4. The reactions were initiated by adding NADPH regenerating system. The final incubation mixtures contained 1 μM test compound, 0.5 mg/mL liver microsomal protein, 1 mM MgCl₂, 1 mM NADP, 1 unit/mL isocitric dehydrogenase and 6 mM isocitric acid in 100 mM potassium phosphate buffer, pH 7.4. After incubation times of 0, 3, 6, 9, 15 and 30 minutes at 37° C., 300 μL of cold acetonitrile (including internal standard) was added to 100 μL incubation mixture to terminate the reaction. Following precipitation and centrifugation, the amount of compound remaining in the samples were determined by LC-MS/MS. Controls of no NADPH regenerating system at zero and 30 minutes were also prepared and analyzed. The compounds of present invention showed good human liver microsome stability determined in the above assay, results are shown in Table 5 below.

TABLE 5
Human liver microsome stability of
the compounds of present invention

| | Clearance of Human |
|---|---|
| Example No | microsome (mL/min/kg) |
| N8 | 16.3 |
| N79 | 18.2 |
| 1 | 6.2 |
| 2 | 9.0 |
| 3 | 9.5 |
| 7 | 10.3 |
| 8 | 7.0 |
| 9 | 9.9 |
| 13 | 6.2 |
| 14 | 6.2 |
| 15 | 6.2 |
| 16 | 8.2 |
| 18 | 10.5 |
| 19 | 7.7 |
| 20 | 10.2 |
| 21 | 10.1 |
| 22 | 6.2 |
| 23 | 7.5 |
| 26 | 6.2 |
| 27 | 6.2 |
| 28 | 6.2 |
| 29 | 9.7 |
| 31 | 7.9 |
| 34 | 10.1 |
| 35 | 8.4 |
| 36 | 6.2 |
| 37 | 8.2 |
| 38 | 6.2 |
| 39 | 6.2 |
| 40 | 6.2 |
| 41 | 6.2 |
| 42 | 6.8 |
| 43 | 6.2 |
| 45 | 10.4 |
| 46 | 6.2 |
| 47 | 6.2 |
| 48 | 7.2 |
| 50 | 6.2 |
| 51 | 7.7 |
| 52 | 6.2 |
| 53 | 7.0 |
| 55 | 8.2 |
| 58 | 9.0 |
| 59 | 9.4 |
| 62 | 6.8 |
| 63 | 6.2 |
| 64 | 7.0 |
| 66 | 9.8 |
| 67 | 10.5 |
| 68 | 9.8 |
| 69 | 8.1 |
| 70 | 8.1 |
| 72 | 7.3 |
| 73 | 6.3 |
| 74 | 9.2 |
| 76 | 6.2 |
| 77 | 6.2 |
| 78 | 6.9 |
| 79 | 8.0 |
| 80 | 6.2 |

### Example 86

### 3T3 In Vitro Phototoxicity Assay

Phototoxicity is defined as a toxic response that is elicited after the first exposure of the skin to certain chemicals and subsequent exposure to light, or that is induced similarly by skin irradiation after systemic administration of a chemical substance. The assay used in this study is designed to detect the phototoxic potential of a chemical by using a simple in vitro cytotoxicity assay with Balb/c 3T3 mouse fibroblasts. The principle of this test is a comparison of the cytotoxicity of a chemical when tested with and without exposure to a non-toxic dose of UVA-light. Cytotoxicity is expressed as a dose dependent reduction of the growth rate of cells as determined by uptake of the vital dye Neutral Red one day after treatment.

### 1. Method

### Preparation of Stock Solution and Dosage of Test Item

A small amount of substance was weighed and formulated freshly in DMSO just before the start of the exposure of the cells. This stock solution or appropriate dilutions with DMSO were added to the cell suspensions to obtain the required final concentrations. All solutions were generally prepared in Eppendorf caps and discarded after use.

### Reference Substance

Chlorpromazine (HCL) (Sigma, Batch/Lot No.: 120M1328V), test concentration: 300 μg/mL, Solvent: PBS/3% DMSO

### Measurement of UV Absorption Spectrum

The absorption spectra as such or with UV-A or with UV-B pre-irradiation were recorded between 240 nm and 400 nm with a Lambda-2 spectral photometer (Perkin Elmer).

| | | | |
|---|---|---|---|
| UV | for UV-A: | Sol 500 with filter H1 | |
| radiation | | Main spectrum: | 315-690 nm |
| sources: | | Irradiance: | approx. 1.67 mW/cm² |
| | | Radiation dose : | approx. 5 J/cm² |
| | for UV-B: | Philips TL 20 W/12 | |
| | | Main spectrum: | 290-320 nm |
| | | Irradiance: | approx. 0.083 mW/cm² |
| | | Radiation dose: | approx. 0.05 J/cm² |

### Determination of Phototoxicity

For this study the Neutral Red uptake (NRU) assay of Borenfreund and Puerner (Borenfreund, E, Puerner J A. Toxicity determined in vitro by morphological alterations and Neutral Red absorption. Toxicology Lett. 1985; 24:119-124) modified according to INVITTOX protocol No 78 (ERGATT/FRAME data bank of in vitro techniques in toxicology. INVITTOX PROTOCOL No 78. 3T3 NRU Phototoxicity Assay. March 1994) has been adapted to examine a possible phototoxic potential of the test item. This assay is based on the active uptake of the Neutral Red dye into the lysosomes of cultured murine fibroblasts. Because lysosomal membranes are known to be a site of action of many phototoxic compounds, this assay can provide a measure of potential for phototoxic injury.

### Preparation of Cell Culture

A murine fibroblasts clone A 31 (ATCC no. CCL 163—passage No. 108) were cultured in 175 cm² tissue culture grade flasks, containing sDMEM (Dulbecco's Minimal Essential Medium, supplemented with 10% fetal calf serum, 2 mM L-glutamine, 100 units/ml Penicillin and 100 μg/ml streptomycin) at 37° C. in a humidified atmosphere of 6% CO₂. Before cells approach confluence they were removed from flasks by trypsinisation. Prior to use in an assay, the cells were transferred to 96-well microtiter plates at a concentration of 1×10⁴ cells/well in 100 μl volumes of sDMEM and allowed to attach for 24 h.

### Exposure to Test Item

For incubation with murine fibroblasts, the test item was diluted in PBS/3% DMSO (detailed concentrations see in results).

Culture medium (Dulbecco's Modified Eagle Medium(DMEM), GlutaMAX (Gibco Ref 21885-025), 10% Fetal Bovine Serum (FBS) (Gibco Ref 10270-106), 1001U/ml Penicillin and 100 μg/mL Streptomycin (Gibco Ref 15140-122)) was removed from the wells and murine fibroblasts were washed with PBS. Afterwards 100 μL of PBS/3% DMSO containing the test item was added and target cells were incubated for 1 h at 37° C. with 6% CO₂.

### UV Exposure

For each test item the microtiter plates were prepared according to Table 6. “UVA plates” were exposed to approx. 5 J/cm² UVA light, the “Dark plates” were kept in the dark and served as cytotoxicity control. Plates with chlorpromazine hydrochloride served as positive control. UV flux was measured with a UV-meter (Dr. Gröbel RM21).

Following UV irradiation, the test item was removed from the wells (one washing step with PBS) and replaced with sDMEM. Target cells were then incubated overnight at 37° C. in 6% CO₂.

TABLE 6
96-well microtiter plate setup

| | 1 | 2 | 3 | 4 | 5 | 6 | 7 | 8 | 9 | 10 | 11 | 12 |
|---|---|---|---|---|---|---|---|---|---|---|---|---|
| A | S1 | S2 | U01 | U02 | U03 | U04 | U05 | U06 | U07 | U08 | S2 | S1 |
| B | S1 | S2 | | | | | | | | | S2 | S1 |
| C | S1 | S2 | | | | | | | | | S2 | S1 |
| D | S1 | S2 | | | | | | | | | S2 | S1 |
| E | S1 | S2 | | | | | | | | | S2 | S1 |
| F | S1 | S2 | | | | | | | | | S2 | S1 |
| G | S1 | S2 | | | | | | | | | S2 | S1 |
| H | S1 | S2 | | | | | | | | | S2 | S1 |

96-Well Microtiter Plates were Prepared as Follows:

Each plate contained wells with cells and solvent but without test item which were either not incubated with Neutral Red solution (0% standard—S1) or were stained with Neutral Red (100% standard—S2) for calculation of the standard cell viability curve. Wells labeled with U01-U08 contained the different test item concentrations.

### Neutral Red Uptake

The ready to use Neutral Red (NR) staining solution was freshly prepared as follows:
0.4% aqueous stock solution was shielded from light and filtered before use to remove NR crystals.
1:40 dilution of the stock solution was then prepared in sDMEM and added to the cells.

After the incubation the wells to be assayed were filled with 100 μL of the sDMEM containing Neutral Red. The target cells were incubated with the NR for 3 h at 37° C. in 6% CO₂.

### Measurement of Neutral Red Uptake

Unincorporated Neutral Red was removed from the target cells and the wells washed with at least 100 μL of PBS. 150 μL of Neutral Red desorb solution (1% glacial acetic acid, 50% ethanol in aqua bidest) was then added to quantitatively extract the incorporated dye. After at least 10 mins of vigorous shaking of the plates on a microtiter plate shaker until Neutral Red has been extracted from the cells and formed a homogeneous solution, the absorption of the resulting colored solution was measured with a SPECTRAmax PLUS microtiter plate reader (Molecular Devices) at 540 nm.

### Calculation of Cell Viability

Cell viability was calculated with the SOFTmax Pro software package (Molecular Devices). First a two-point standard curve (0% and 100% viability) was calculated with the linear curve fit option of the program based on the following formula:

$$Y = A + ( B \times X )$$

(A=y-intercept of the line; B=slope of the line;
0% cell viability=cells with solvent, but without test item and Neutral Red;
100% cell viability=cells with solvent and Neutral Red, but without test item)

By this means the viability of the cells incubated with increasing concentrations of the test chemical was calculated. Chlorpromazine (HCl) served as positive control in the experiment.

### Calculation of IC₅₀ Values

All calculations were performed with the SOFTmax Pro analysis software package
(Molecular Devices—for details see:
http://www.mbl.edu/jbpc/files/2014/05/SoftMax_Pro_User_Guide.pdf)

### Calculation of Discrimination Factor for Phototoxicity

For evaluation of phototoxic potential, the IC₅₀ values determined with and without UV exposure were compared.

$$Factor = {IC}_{50} ( - UV ) / {IC}_{50} ( + UV )$$

For discrimination between phototoxic and non-phototoxic test chemicals a cut-off factor of >5 was applied (Liebsch M, Spielmann H, Balls M, Brand M, Döring B, Dupuis J, Holzhiiter HG, Klecak G, L. Eplattenier H, Lovell W, Maurer T, Moldenhauer F, Moore L, Pape W, Pfannenbecker U, Potthast J M, De Silva O, Steiling W, Willshaw A. First results of the EC/COLIPA Validation Project. In Vitro Phototoxicity Testing. In: In Vitro Skin Toxicology: Irritation, Phototoxicity, Sensitization; Vol. 10. Alternative Methods in Toxicology, -Eds. Rougier A, Maibach HI, Goldberg AM; Mary Ann Liebert Publ.: New York, USA 1994, pp. 243-251).

Test items which are not cytotoxic to murine fibroblasts even at the highest concentrations tested, but show a strong dose dependent decrease in cell viability after UV exposure are considered also phototoxic (Spielmann H, Balls M, Dupuis J, Pape W J W, Pechovitch G, Silva DeO, Holzhütter, H G, Clothier R, Desolle P, Gerberick F, Liebsch M, Lowell W W, Maurer T, Pfannenbecker U, Potthast J M, Csato M, Sladowski D, Steiling W, Brantom P. The international EU/COLIPA in vitro phototoxicity validation study: Results of phase II (blind trial). Part 1: The 3T3 NRU phototoxicity test. Toxicology in Vitro 1998, 12: 305-327).

The test results were shown below, the compounds of this invention showed very good phototoxicity profile.

TABLE 7
The 3T3 test results for the compound of this invention

| Example No | Phototoxicity factor | IC₅₀ (UV-A) (μg/mL) |
|---|---|---|
| 3 | 1 | >100 |
| 20 | 1 | >100 |
| 63 | 1 | >100 |

### Example 87

### Embryonic Stem Cell Test

The in vitro mouse Embryonic Stem Cell Test (mEST) assay is an implemented routine assay at Roche. The original EST was developed by Horst Spielmann and his group in 1997 as an in vitro model for the screening of embryotoxicity, based on a blastocyst-derived permanent embryonic mouse ESC (mESC) D3 cell line derived from mouse 129 strains and was validated by European Centre for the Validation of Alternative Methods (ECVAM).

We further optimized and modified the approach allowing for the application of the assay to pharmaceutical compounds.

### Biological Endpoint and Endpoint Measurement:

The cytotoxicity (inhibition of growth) of 3T3 fibroblasts, which represents differentiated cells and the cytotoxicity of undifferentiated embryonic stem cells (D3) after 10 days of substance treatment serve as two assay endpoints. This is determined by the use of dehydrogenase enzymes, which are present in the intact mitochondria of living cells to convert yellow soluble substrate 3-(4,5-dimethythiazol-2-yl)-2,5-diphenyl tetrazolium bromide (MTT) into a dark blue insoluble formazan product, which gets sequestered within the cells and is detected quantitatively using an absorbance reader (570 nm) after solubilizing the cell membrane.

The third endpoint is the inhibition of differentiation of ES cells into myocards which are cardiac muscle cells after 10 days of treatment. The beating of this cells is evaluated by microscopy.

### Materials and Reagents

mESC cell: ES-D3 [D3] (ATCC® CRL-1934™)
mouse Fibroblasts: BALB/3T3 clone A31 (ATCC® CCL-163™)
Balb/c 3T3 cell clone A31: American Type Culture Collection (ATCC) Cat No CCL-163
ES-D3 (D3): American Type Culture Collection (ATCC) Cat No CRL-1934
m-LIF: Sigma, Cat No L5158-5UG
NEAA (100×): Gibco, Cat No 11140-035
Trypan blue 0.04%: Gibco, Cat No T10282
MTT: Tocris Bioscience, Cat No 5224/500
5-Fluorouracil: Sigma, Cat No F-6627-5G
Penicillin/Streptomycin: Gibco, Cat No 15140-122
PBS (—CaCl₂)/—MgCl2): Gibco, Cat No 14190-094
FCS: Hyclone, Cat No SH30070.03
DMEM with Glucose, Glutamine, NaHCO₃: Gibco, Cat No 41966-029

### Method

### Preparations

### Media and Endpoint Assay Solutions

| 3T3 | D3 |
|---|---|
| Culture Media: | |
| 10% FCS | 20% FCS |
| 4 mM Glutamine | 2 mM Glutamine |
| 50 U/mL Penicillin | 50 U/mL Penicillin |
| 50 μg/mL Streptomycin | 50 μg/mL Streptomycin |
| | 1% NAA |
| | 0.1 mM β-ME |
| | 1000 U/mL m-LIF (only added |
| | separately to subculture) |
| Assay Media: | |
| 10% FCS | 20% FCS |
| 4 mM Glutamine | 2 mM Glutamine |
| 50 U/mL Penicillin | 50 U/mL Penicillin |
| 50 μg/mL Streptomycin | 50 μg/mL Streptomycin |
| | 1% NAA |
| | 0.1 mM β-ME |
| Media for Freezing Cells: | |
| 20% FCS | 40% FCS |
| 4 mM Glutamine | 2 mM Glutamine |
| 50 U/mL Penicillin | 50 U/ml Penicillin |
| 50 μg/mL Streptomycin | 50 μg/ml Streptomycin |
| 7% DMSO | 1% NAA |
| | 0.1 mM β-ME |
| | 7% DMSO |

### β-Mercapthoethanol (β-ME) (10 mM)

17.5 μL β-ME added to 25 mL of PBS
Store at 4° C. for max. 1 week

### FCS

Thaw FCS once up by water bath (37° C.) and make aliquots of 100 mL, 50 mL and 25 mL.
Avoid multiple thawing
Store at −20° C.

### MTT-Solution

5 mg MTT/ml PBS
Use a sterile Filter from Millipore and make aliquots of 8 mL and 4 mL
Store at −20° C.

### MTT-Desorb-Solution

20% SDS solved in water/DMF, 1:1, adjust the pH to 4.5 with acetic acid

### Test Compounds

Stock solution: 200 mM
Solvent: 100% DMSO

### Differentiation Assay

### Day 0

1) Cell passage with trypsin 0.05% EDTA for D3 cells.
2) Assembly of the cell suspension: dilute cells to 2.5×10⁴/mL with 18 ml media (for each test) in 50 mL Falcon tubes.
3) Prepare the petri dishes (PD): add 5-10 mL sterile Dulbecco's PBS (Gibco) into each dish bottom, distribute over the whole dish.
4) Dilution series of test compound in Eppendorf tubes: add 5 μL of compound (1:400 dilution) and 5 μL control solution (DMSO) to 2 mL cell suspension, vortex.
5) Preparation of hanging drops in petridishes: vortex tube, aspirate suspension with automatic pipette and multi-dispense 20 μL drops onto the lid of the petri dish, add 2 mL in total in a concentric circle of drops (˜100 drops); quickly but smoothly turn the cover and put on PD; incubate for 3 days at 37° C./5% CO₂.

### DAY 3

1) Dilution series of compound in 14 mL PP tubes
6 tubes for the concentrations; fill with 5 mL assay media
1 tube for DMSO (solvent control) fill with 5 mL assay media
2) Dilution series of compound in Eppendorf tubes
Add 12.5 μL of compound (1:400 dilution) and 12.5 μL control solution, vortex
3) Transfer of embryoid bodies in a bacterial Petri dish
Carefully turn PD lid, check the drops for fungus contamination
Rinse several times the drops down with 5 mL of the prepared solution
Transfer in a bacterial petri dish
   Incubate for 3 days at 37° C./5% CO₂

### Day 5

1) Dilution series of compound in 50 mL tubes
6 tubes for the concentrations; fill with 25 mL assay media
1 tube for DMSO (solvent control) fill with 25 mL media
2) Dilution series of compound in 1.5 mL tubes
Add 62.5 μL of compound (1:400 dilution) and control solution (DMSO), vortex
3) Preparation of 96 well plates
2 plates for each compound, see Compound-plate-Layout
Add 220 μL media/compound/solvent mix in all 96 wells
Start with low concentration
4) Pipetting of embryoid bodies
Visually control the embryoid bodies in the petri dish
With a 25 μL tip, pipette one embryoid body in each well
Check visually the plate to ensure that at least one embryoid body is present in each well
Incubate for 3 days at 37° C./5% CO₂

### DAY 10

Visualize each well with microscope for beating myocard cells
Assay media and DMSO controls should show at least 80% of beating cardiomyocyte cells (see acceptance criteria)

### Cytotoxicity Assay

Stock solution with a concentration of 0.2 mol/L is created for all substances. Test substances are diluted in DMSO solution.

### DAY 0

1) Create cell suspension for D3 and 3T3 cell lines
2) 2.5×10⁴ cells/mL for 3T3, 1.5×10⁴ cells/mL for D3 cells
3) Pipetting of 200 μL medium in the outer wells of a 96-well multi well plate (blanks) 4) Add 50 μL cell suspension into to the remaining inner wells of the 96-well multi well plate (samples)
5) Incubate for 2 h at 37° C./5% CO₂ to let the cell adhere
6) Pipetting of the test substances or DMSO controls Create concentrations of 2 mL medium and 6.67 μL test substance in a 5 mL tube
7) Add 150 μL/well of the solution into the sample wells (200 μL/well in total) 8) Incubate for 3 days at 37° C./5% CO₂

### DAY 3, 5 AND 7

1) Dilute 2 mL of the medium (3T3 or D3 cell medium) with adding 5 μL of test substance (or DMSO control) (1:400) in a mL tube
2) Remove the medium with a vacuum pump without damaging of the cell layer on the bottom
3) Add 200 μL of the diluted test substances (and DMSO controls) into to the appropriate sample wells

### Incubation:

Day 3: 2 days at 37° C./5% CO₂
Day 5: 2 days at 37° C./5% CO₂
Day 7: 3 days at 37° C./5% CO₂

### DAY 10

Preliminary observe cellular changes, substance precipitation or any other effects visually under the light microscope

### MTT-Measurements:

1) Create final MTT solution by adding 4 mL MTT into 40 mL DMEM and warm up to 37° C.
2) Remove medium out of the 96-well plates by discarding the medium carefully 3) Add 200 μL of MTT solution to each well with a multiwall pipet
4) Incubate the plates for 3h at 37° C./5% CO₂
5) Warm up the MTT-Desorb solution to 37° C.
6) Remove the MTT-solution carefully
7) Add 130 μL of MTT-Desorb solution into each well and incubate the plates for 30 min at 37° C. in the incubator, then put the plates for at least 2-3 hours on a plate shaker
8) Measure the absorption on a plate reader at 570 nm

### Acceptance Criteria

Differentiation Endpoint: at least 80% of beating cardiomyocytes in a total assay needed for acceptance of a valid assay
Cytotoxicity Endpoints:
Acceptable ranges of DMSO control and POS control and the determination of OD values of D3 (about 1.8-2.2) and 3T3 (0.8-1.0) should be in their appropriate ranges

### Data Analysis

### Differentiation Endpoint:

Determination of the total number of beating cardiomyocytes (at least one beating cardiomyocyte per well=one positive count, no beating cardiomyocytes per well=negative count), normalization to the positive DMSO controls

### Cytotoxicity Endpoints:

Determination of the mean values of the OD 570 of the blanks (value indicates the adhesion of the dye to plastic material and residual amount of medium). Subtract this value from sample values and continue to calculate with the corrected values.
Determination of the mean values of the OD 570 of the treated sample wells. Determination of the mean values of the OD 570 of the solvent control wells are set as 100%. The Viability is calculated in % normalized to the DMSO solvent control.

### Prediction Model

Data files of optical densities (OD570) generated by a microplate reader were copied into an EXCEL spreadsheet. Mean OD values, standard deviations and viabilities were calculated automatically. The following endpoints from the assays could be calculated graphically from the concentration-response curve in the spreadsheet:
IC50 D3—the concentration of test substance at which 50% of D3 cells have died
IC50 3T3—the concentration of test substance at which 50% of the 3T3 cells have died.
ID50 D3—the concentration of test substance at which there is a 50% reduction in the differentiation of D3 cells into contracting cardiomyocytes.

The IC50 values of the D3 and 3T3 cells from the cytotoxicity assay and the ID50 of the D3 differentiation assay were entered into the statistical evaluation developed from the modified prediction model used by Scholz et al. 1999a:

$$D12\_ 3 = \frac{lg IC 50 D 3 + lg IC 50 \; 3 T 3}{2} - lg ID 50$$

D12_3<0.5 means “negative”
D12_3>0.6 means “positive”

Predictive scores between 0.5 and 0.6 are labelled borderline results.

Inconclusive results are also possible, for example, if solubility limits the dose ranges tested to an extent that no IC50 or ID50 values can be determined for one or more dose response curves (Withlow et al. 2007)

TABLE 8
mEST results for the compound of this invention

| Example No | Predictive scores D12_3 |
|---|---|
| 18 | 0.11 |
| 19 | 0.05 |
| 20 | 0.49 |
| 63 | 0.43 |

### Example 88

### Single Dose Pharmacokinetics (PK) Study in Male Wister-Han Rats

Pharmacokinetic properties of selected compounds were assessed by single dose PK studies in Male Wister-Han Rats (vendor: Beijing Vital River Laboratory Animal Technology Co., Ltd). Briefly, two groups of animals were administered a single dose of respective compound intravenously (IV, bolus) at 2 mg/kg or orally (PO, by gavage) at 10 mg/kg. Blood samples (approximately 150 μL) were collected via Jugular vein at 5 min (only for IV), 15 min, 30 min, 1 h, 2 h, 4 h, 7 h and 24 h post-dose. Blood samples were placed into tubes containing EDTA-K2 anticoagulant and centrifuged at 3000 rpm for 15 min at 4° C. to separate plasma from the samples. After centrifugation, the resulting plasma was transferred to clean tubes for bioanalysis with LC/MS/MS. The pharmacokinetic parameters were calculated using non-compartmental analysis. The volume of distribution (Vss), half-life (T_{1/2}) and clearance (CL) were obtained based on the plasma concentration-time curve after IV dose. The peak concentration (Cₘₐₓ) was recorded directly from experimental observations after PO dose. The area under the plasma concentration-time curve (AUC₀₋ₗₐₛₜ) was calculated using the linear trapezoidal rule up to the last detectable concentration. The bioavailability (F) was calculated based on the dose normalized AUC₀₋ₗₐₛₜ after IV and PO dose.

The Vss of a drug represents the degree to which a drug is distributed in body tissue rather than the plasma. Vss is directly proportional with the amount of drug distributed into tissue. A higher Vss indicates a greater amount of tissue distribution.

Results of PK parameters following IV and PO administration are given in Table 9.

TABLE 9
PK parameters for the compounds of this invention

| Example | PO Cₘₐₓ | PO AUC₀₋ₗₐₛₜ | IV AUC₀₋ₗₐₛₜ | CL | Vss | T_{1/2} | F |
|---|---|---|---|---|---|---|---|
| No | (ng/mL) | (h × ng/mL) | (h × ng/mL) | (mL/min/kg) | (L/kg) | (h) | (%) |
| 1 | 474 | 7266 | 1286 | 24 | 11.2 | 6.9 | 113 |
| 19 | 102 | 1410 | 887 | 34 | 16.0 | 8.9 | 32 |
| 20 | 1140 | 16508 | 5477 | 6 | 1.5 | 3.3 | 60 |
| 23 | 1600 | 27460 | 7548 | 3.7 | 2.7 | 10.5 | 73 |
| 63 | 22200 | 394107 | 80714 | 0.26 | 0.38 | 18.3 | 98 |

1. A compound of formula (I), 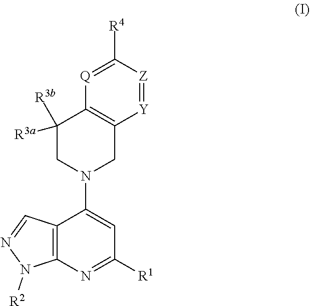
wherein:
R¹ is H or C₁₋₆alkyl;
R² is C₁₋₆alkyl;
R^{3a} is H or C₁₋₆alkyl;
R^{3b} is H or C₁₋₆alkyl;
R⁴ is (C₁₋₆alkylpiperazinyl)C₁₋₆alkoxy, (C₁₋₆alkyl)₂piperazin-4-iumyl, (formylpiperazinyl)C₁₋₆alkoxy, 1,2,3,4,6,7,8,8a-octahydropyrrolo[1,2-a]pyrazinyloxy, 2-oxo-1-oxa-3,7-diazaspiro[4.4]nonanyl, 2-oxo-1-oxa-3,8-diazaspiro[4.5]decanyl, 3,8-diazabicyclo[3.2.1]octanyl, 3,9-diazaspiro[5.5]undecanyl, 3-oxo-1,5,6,7,8,8a-hexahydroimidazo[1,5-a]pyrazinyl, 3-oxo-2,7-diazaspiro[4.4]nonanyl, 3-oxo-2,8-diazaspiro[4.5]decanyl, 3-oxo-3a,4,5,6,7,7a-hexahydro-1H-pyrrolo[3,4-c]pyridinyl, 4,7-diazaspiro[2.5]octanyl, 4-oxo-1,2,3,3a,6,6a-hexahydropyrrolo[3,4-c]pyrrolyl, 4-piperidinylazetidinyloxy, 5-oxa-2,8-diazaspiro[3.5]nonanyl, 7-oxo-2,6-diazaspiro[3.4]octanyl, piperazinyl, piperazinylC₁₋₆alkoxy, piperidinyl, piperidinyloxy, or pyrrolidinyl,
wherein:
piperazinyl is unsubstituted or substituted by (C₁₋₆alkyl)₂aminoC₁₋₆alkyl, 1,2,3,4-tetrahydro-2,6-naphthyridinyl, C₁₋₆alkoxyC₁₋₆alkyl, C₁₋₆alkyl, C₁₋₆alkylimidazolylC₁₋₆alkyl, hydroxyC₁₋₆alkyl, morpholinylcarbonyl, piperidinylcarbonyl, pyrrolidinylcarbonyl or pyrrolidinylsulfonyl, piperidinyl is once or twice substituted by substituents independently selected from halogen, amino, amino(hydroxy)piperidinyl, amino(C₁₋₆alkoxy)pyrrolidinyl, aminoazetidinyl, C₁₋₆alkyl, C₁₋₆alkoxy, C₁₋₆alkyl)₂amino, hydroxy, ((C₁₋₆alkyl)₂amino)C₁₋₆alkyl and piperazinyl; and
pyrrolidinyl is twice or three times substituted by substituents independently selected from halogen, amino, hydroxy, C₁₋₆alkyl and C₁₋₆alkoxy;
Q and Z are independently selected from CH and N; and
Y is CH;
or a pharmaceutically acceptable salt thereof.

2. A compound of formula (Ia), 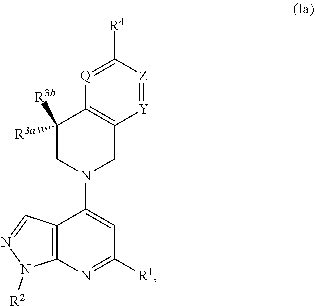
wherein:
R¹ is H or C₁₋₆alkyl;
R² is C₁₋₆alkyl;
R^{3a} is H or C₁₋₆alkyl;
R^{3b} is H or C₁₋₆alkyl;
R⁴ is (C₁₋₆alkylpiperazinyl)C₁₋₆alkoxy, (C₁₋₆alkyl)₂piperazin-4-iumyl, (formylpiperazinyl)C₁₋₆alkoxy, 1,2,3,4,6,7,8,8a-octahydropyrrolo[1,2-a]pyrazinyloxy, 2-oxo-1-oxa-3,7-diazaspiro[4.4]nonanyl, 2-oxo-1-oxa-3,8-diazaspiro[4.5]decanyl, 3,8-diazabicyclo[3.2.1]octanyl, 3,9-diazaspiro[5.5]undecanyl, 3-oxo-1,5,6,7,8,8a-hexahydroimidazo[1,5-a]pyrazinyl, 3-oxo-2,7-diazaspiro[4.4]nonanyl, 3-oxo-2,8-diazaspiro[4.5]decanyl, 3-oxo-3a,4,5,6,7,7a-hexahydro-1H-pyrrolo[3,4-c]pyridinyl, 4,7-diazaspiro[2.5]octanyl, 4-oxo-1,2,3,3a,6,6a-hexahydropyrrolo[3,4-c]pyrrolyl, 4-piperidinylazetidinyloxy, 5-oxa-2,8-diazaspiro[3.5]nonanyl, 7-oxo-2,6-diazaspiro[3.4]octanyl, piperazinyl, piperazinylC₁₋₆alkoxy, piperidinyl, piperidinyloxy, or pyrrolidinyl,
wherein:
piperazinyl is unsubstituted or substituted by (C₁₋₆alkyl)₂aminoC₁₋₆alkyl, 1,2,3,4-tetrahydro-2,6-naphthyridinyl, C₁₋₆alkoxyC₁₋₆alkyl, C₁₋₆alkyl, C₁₋₆alkylimidazolylC₁₋₆alkyl, hydroxyC₁₋₆alkyl, morpholinylcarbonyl, piperidinylcarbonyl, pyrrolidinylcarbonyl or pyrrolidinylsulfonyl;
piperidinyl is once or twice substituted by substituents independently selected from halogen, amino, amino(hydroxy)piperidinyl, amino(C₁₋₆alkoxy)pyrrolidinyl, aminoazetidinyl, C₁₋₆alkyl, C₁₋₆alkoxy, C₁₋₆alkyl)₂amino, hydroxy, ((C₁₋₆alkyl)₂amino)C₁₋₆alkyl and piperazinyl; and
pyrrolidinyl is twice or three times substituted by substituents independently selected from halogen, amino, hydroxy, C₁₋₆alkyl and C₁₋₆alkoxy;
Q and Z are independently selected from CH and N; and
Y is CH;
or a pharmaceutically acceptable salt thereof.

3. A compound according to claim 1, wherein Q and Z are not N simultaneously.

4. A compound according to claim 1, wherein R⁴ is (1-C₁₋₆alkylpiperazin-2-yl)C₁₋₆alkoxy, (C₁₋₆alkyl)₂piperazin-4-ium-1-yl, (1-formylpiperazin-2-yl)C₁₋₆alkoxy, 1,2,3,4,6,7,8,8a-octahydropyrrolo[1,2-a]pyrazin-7-yloxy, 2-oxo-1-oxa-3,7-diazaspiro[4.4]nonan-3-yl, 2-oxo-1-oxa-3,8-diazaspiro[4.5]decan-3-yl, 3,8-diazabicyclo[3.2.1]octan-8-yl, 3,9-diazaspiro[5.5]undecan-3-yl, 3-oxo-1,5,6,7,8,8a-hexahydroimidazo[1,5-a]pyrazin-2-yl, 3-oxo-2,7-diazaspiro[4.4]nonan-2-yl, 3-oxo-2,8-diazaspiro[4.5]decan-2-yl, 3-oxo-3a,4,5,6,7,7a-hexahydro-1H-pyrrolo[3,4-c]pyridin-2-yl, 4-oxo-1,2,3,3a,6,6a-hexahydropyrrolo[3,4-c]pyrrol-5-yl, 4-piperidylazetidin-3-yloxy, 5-oxa-2,8-diazaspiro[3.5]nonan-2-yl, 7-oxo-2,6-diazaspiro[3.4]octan-6-yl, piperazin-1-yl, 1,2,3,4-tetrahydro-2,6-naphthyridin-7-yl-piperazin-1-yl, (1-C₁₋₆alkylimidazol-4-ylC₁₋₆alkyl)piperazin-1-yl, (hydroxyC₁₋₆alkyl)piperazin-1-yl, C₁₋₆alkyl-piperazin-1-yl, (morpholin-2-ylcarbonyl)piperazin-1-yl, (C₁₋₆alkyl)₂aminoC₁₋₆alkyl-piperazin-1-yl, piperidin-4-ylcarbonyl-piperazin-1-yl, pyrrolidin-2-ylcarbonyl-piperazin-1-yl, pyrrolidin-3-ylsulfonyl-piperazin-1-yl, (C₁₋₆alkoxyC₁₋₆alkyl)piperazin-1-yl, piperazin-1-ylC₁₋₆alkoxy, piperazin-2-ylC₁₋₆alkoxy, (3-amino-4-C₁₋₆alkoxy-pyrrolidin-1-yl)-1-piperidinyl, 3-aminoazetidin-1-yl-1-piperidinyl, (4-amino-3-hydroxy-1-piperidinyl)-1-piperidinyl, 4-hydroxy-4-((C₁₋₆alkyl)₂amino)C₁₋₆alkyl-1-piperidinyl, piperazin-1-yl-1-piperidinyl, amino-1-piperidinyl, 4-amino-4-C₁₋₆alkyl-1-piperidinyl, (C₁₋₆alkyl)₂amino-1-piperidinyl, 4-amino-3-methoxy-1-piperidinyl, 4-amino-3-halo-1-piperidinyl, 3-amino-4-C₁₋₆alkoxy-pyrrolidin-1-yl, 4-amino-3-hydroxy-pyrrolidin-1-yl, 3-amino-4-halo-pyrrolidin-1-yl, or 4-amino-3-hydroxy-3-C₁₋₆alkyl-pyrrolidin-1-yl.

5. A compound according to claim 4, wherein R⁴ is 3-amino-4-C₁₋₆alkoxy-pyrrolidin-1-yl, 4-amino-3-hydroxy-pyrrolidin-1-yl, 3-aminoazetidin-1-yl-1-piperidinyl, 4-amino-3-C₁₋₆alkoxy-1-piperidinyl, 5-oxa-2,8-diazaspiro[3.5]nonan-2-yl, (hydroxyC₁₋₆alkyl)piperazin-1-yl, piperazin-1-yl or piperazin-2-ylC₁₋₆alkoxy.

6. A compound according to claim 5, wherein R⁴ is 3-amino-4-methoxy-pyrrolidin-1-yl, 4-amino-3-hydroxy-pyrrolidin-1-yl, 4-(3-aminoazetidin-1-yl)-1-piperidinyl, 5-oxa-2,8-diazaspiro[3.5]nonan-2-yl, 3-(hydroxymethyl)piperazin-1-yl, piperazin-1-yl or piperazin-2-ylmethoxy.

7. A compound according to claim 5, wherein R⁴ is 3-amino-4-C₁₋₆alkoxy-pyrrolidin-1-yl or piperazin-2-ylC₁₋₆alkoxy.

8. A compound according to claim 7, wherein R⁴ is 3-amino-4-methoxy-pyrrolidin-1-yl or piperazin-2-ylmethoxy.

9. A compound according to claim 1, wherein R^{3b} is H.

10. A compound according to claim 9, wherein R^{3a} is C₁₋₆alkyl.

11. A compound according to claim 10, wherein R^{3a} is methyl.

12. A compound according to claim 10, wherein R² is methyl.

13. A compound according to claim 1, wherein:
R¹ is H or C₁₋₆alkyl;
R² is C₁₋₆alkyl;
R^{3a} is C₁₋₆alkyl;
R^{3b} is H; and
R⁴ is 3-amino-4-C₁₋₆alkoxy-pyrrolidin-1-yl, 4-amino-3-hydroxy-pyrrolidin-1-yl, 3-aminoazetidin-1-yl-1-piperidinyl, 4-amino-3-C₁₋₆alkoxy-1-piperidinyl, 5-oxa-2,8-diazaspiro[3.5]nonan-2-yl, (hydroxyC₁₋₆alkyl)piperazin-1-yl, piperazin-1-yl or piperazin-2-ylC₁₋₆alkoxy;
with the proviso that Q and Z are not N simultaneously;
or a pharmaceutically acceptable salt thereof.

14. A compound according to claim 13, wherein
R¹ is H or methyl;
R² is methyl;
R^{3a} is methyl; and
R⁴ is 3-amino-4-methoxy-pyrrolidin-1-yl, 4-amino-3-hydroxy-pyrrolidin-1-yl, 4-(3-aminoazetidin-1-yl)-1-piperidinyl, 5-oxa-2,8-diazaspiro[3.5]nonan-2-yl, 3-(hydroxymethyl)piperazin-1-yl, piperazin-1-yl or piperazin-2-ylmethoxy;
or a pharmaceutically acceptable salt thereof.

15. A compound according to claim 1, wherein:
R¹ is H or C₁₋₆alkyl;
R² is C₁₋₆alkyl;
R^{3a} is C₁₋₆alkyl;
R^{3b} is H;
R⁴ is 3-amino-4-C₁₋₆alkoxy-pyrrolidin-1-yl or piperazin-2-ylC₁₋₆alkoxy;
Q is CH or N; and
Z is CH;
or a pharmaceutically acceptable salt thereof.

16. A compound according to claim 15, wherein
R¹ is H or methyl;
R² is methyl;
R^{3a} is methyl;
R^{3b} is H; and
R⁴ is 3-amino-4-methoxy-pyrrolidin-1-yl or piperazin-2-ylmethoxy;
or a pharmaceutically acceptable salt thereof.

17. A compound selected from:
(4R)-4-methyl-2-(1-methylpyrazolo[3,4-b]pyridin-4-yl)-6-piperazin-1-yl-3,4-dihydro-1H-isoquinoline;
(4R)-2-(1,6-dimethylpyrazolo[3,4-b]pyridin-4-yl)-4-methyl-6-piperazin-1-yl-3,4-dihydro-1H-isoquinoline;
2-(1-methylpyrazolo[3,4-b]pyridin-4-yl)-6-piperazin-1-yl-3,4-dihydro-1H-isoquinoline;
3-[4-methyl-2-(1-methylpyrazolo[3,4-b]pyridin-4-yl)-3,4-dihydro-1H-isoquinolin-6-yl]-3,9-diazaspiro[5.5]undecane;
3-[2-(1,6-dimethylpyrazolo[3,4-b]pyridin-4-yl)-4-methyl-3,4-dihydro-1H-isoquinolin-6-yl]-3,9-diazaspiro[5.5]undecane;
3-[4,4-dimethyl-2-(1-methylpyrazolo[3,4-b]pyridin-4-yl)-1,3-dihydroisoquinolin-6-yl]-3,9-diazaspiro[5.5]undecane;
4-piperidyl-[4-[(4R)-4-methyl-2-(1-methylpyrazolo[3,4-b]pyridin-4-yl)-3,4-dihydro-1H-isoquinolin-6-yl]piperazin-1-yl]methanone;
pyrrolidin-2-yl-[4-[(4R)-4-methyl-2-(1-methylpyrazolo[3,4-b]pyridin-4-yl)-3,4-dihydro-1H-isoquinolin-6-yl]piperazin-1-yl]methanone;
7-[4-[(4R)-4-methyl-2-(1-methylpyrazolo[3,4-b]pyridin-4-yl)-3,4-dihydro-1H-isoquinolin-6-yl]piperazin-1-yl]-1,2,3,4-tetrahydro-2,6-naphthyridine;
(4R)-4-methyl-2-(1-methylpyrazolo[3,4-b]pyridin-4-yl)-6-(4-piperazin-1-yl-1-piperidyl)-3,4-dihydro-1H-isoquinoline;
(3R,4R)-4-methoxy-1-[1-[(4R)-4-methyl-2-(1-methylpyrazolo[3,4-b]pyridin-4-yl)-3,4-dihydro-1H-isoquinolin-6-yl]-4-piperidyl]pyrrolidin-3-amine;
morpholin-2-yl-[4-[(4R)-4-methyl-2-(1-methylpyrazolo[3,4-b]pyridin-4-yl)-3,4-dihydro-1H-isoquinolin-6-yl]piperazin-1-yl]methanone;
4-[(dimethylamino)methyl]-1-[(4R)-4-methyl-2-(1-methylpyrazolo[3,4-b]pyridin-4-yl)-3,4-dihydro-1H-isoquinolin-6-yl]piperidin-4-ol;
4-[(dimethylamino)methyl]-1-[(4R)-2-(1,6-dimethylpyrazolo[3,4-b]pyridin-4-yl)-4-methyl-3,4-dihydro-1H-isoquinolin-6-yl]piperidin-4-ol;
(4R)-4-methyl-6-(3-methylpiperazin-1-yl)-2-(1-methylpyrazolo[3,4-b]pyridin-4-yl)-3,4-dihydro-1H-isoquinoline;
2-[(4R)-4-methyl-2-(1-methylpyrazolo[3,4-b]pyridin-4-yl)-3,4-dihydro-1H-isoquinolin-6-yl]-2,8-diazaspiro[4.5]decan-3-one;
cis-5-[(4R)-4-methyl-2-(1-methylpyrazolo[3,4-b]pyridin-4-yl)-3,4-dihydro-1H-isoquinolin-6-yl]-1,2,3,3a,6,6a-hexahydropyrrolo[3,4-c]pyrrol-4-one;
(4R)-4-methyl-2-(1-methylpyrazolo[3,4-b]pyridin-4-yl)-6-[[(2R)-piperazin-2-yl]methoxy]-3,4-dihydro-1H-isoquinoline;
(4R)-4-methyl-2-(1-methylpyrazolo[3,4-b]pyridin-4-yl)-6-[[(2S)-piperazin-2-yl]methoxy]-3,4-dihydro-1H-isoquinoline;
(3R,4R)-4-methoxy-1-[(4R)-4-methyl-2-(1-methylpyrazolo[3,4-b]pyridin-4-yl)-3,4-dihydro-1H-isoquinolin-6-yl]pyrrolidin-3-amine;
2-[(4R)-4-methyl-2-(1-methylpyrazolo[3,4-b]pyridin-4-yl)-3,4-dihydro-1H-isoquinolin-6-yl]-5-oxa-2,8-diazaspiro[3.5]nonane;
(4R)-2-(1-ethylpyrazolo[3,4-b]pyridin-4-yl)-4-methyl-6-piperazin-1-yl-3,4-dihydro-1H-isoquinoline;
(3R,4R)-4-methoxy-1-[(4R)-2-(1,6-dimethylpyrazolo[3,4-b]pyridin-4-yl)-4-methyl-3,4-dihydro-1H-isoquinolin-6-yl]pyrrolidin-3-amine;
(8aR)-2-[(4R)-4-methyl-2-(1-methylpyrazolo[3,4-b]pyridin-4-yl)-3,4-dihydro-1H-isoquinolin-6-yl]-1,5,6,7,8,8a-hexahydroimidazo[1,5-a]pyrazin-3-one;
(8aS)-2-[(4R)-4-methyl-2-(1-methylpyrazolo[3,4-b]pyridin-4-yl)-3,4-dihydro-1H-isoquinolin-6-yl]-1,5,6,7,8,8a-hexahydroimidazo[1,5-a]pyrazin-3-one;
3-[(4R)-4-methyl-2-(1-methylpyrazolo[3,4-b]pyridin-4-yl)-3,4-dihydro-1H-isoquinolin-6-yl]-1-oxa-3,8-diazaspiro[4.5]decan-2-one;
(4R)-6-(3,8-diazabicyclo[3.2.1]octan-8-yl)-4-methyl-2-(1-methylpyrazolo[3,4-b]pyridin-4-yl)-3,4-dihydro-1H-isoquinoline;
1-[1-[(4R)-4-methyl-2-(1-methylpyrazolo[3,4-b]pyridin-4-yl)-3,4-dihydro-1H-isoquinolin-6-yl]-4-piperidyl]azetidin-3-amine;
trans-4-amino-1-[1-[(4R)-4-methyl-2-(1-methylpyrazolo[3,4-b]pyridin-4-yl)-3,4-dihydro-1H-isoquinolin-6-yl]-4-piperidyl]piperidin-3-ol;
(4R)-2-(1,6-dimethylpyrazolo[3,4-b]pyridin-4-yl)-4-methyl-6-[[(2R)-piperazin-2-yl]methoxy]-3,4-dihydro-1H-isoquinoline;
(4R)-2-(1,6-dimethylpyrazolo[3,4-b]pyridin-4-yl)-4-methyl-6-[[(2S)-piperazin-2-yl]methoxy]-3,4-dihydro-1H-isoquinoline;
(4R)-4-methyl-2-(1-methylpyrazolo[3,4-b]pyridin-4-yl)-6-(4-pyrrolidin-3-ylsulfonylpiperazin-1-yl)-3,4-dihydro-1H-isoquinoline;
6-[(4R)-4-methyl-2-(1-methylpyrazolo[3,4-b]pyridin-4-yl)-3,4-dihydro-1H-isoquinolin-6-yl]-2,6-diazaspiro[3.4]octan-7-one;
3-[(4R)-4-methyl-2-(1-methylpyrazolo[3,4-b]pyridin-4-yl)-3,4-dihydro-1H-isoquinolin-6-yl]-1-oxa-3,7-diazaspiro[4.4]nonan-2-one;
(3R,4R)-4-amino-3-methyl-1-[(4R)-4-methyl-2-(1-methylpyrazolo[3,4-b]pyridin-4-yl)-3,4-dihydro-1H-isoquinolin-6-yl]pyrrolidin-3-ol;
(4R)-4-methyl-6-[4-[(1-methylimidazol-4-yl)methyl]piperazin-1-yl]-2-(1-methylpyrazolo[3,4-b]pyridin-4-yl)-3,4-dihydro-1H-isoquinoline;
N,N-dimethyl-2-[4-[(4R)-4-methyl-2-(1-methylpyrazolo[3,4-b]pyridin-4-yl)-3,4-dihydro-1H-isoquinolin-6-yl]piperazin-1-yl]ethanamine;
(4R)-6-(4,4-dimethylpiperazin-4-ium-1-yl)-4-methyl-2-(1-methylpyrazolo[3,4-b]pyridin-4-yl)-3,4-dihydro-1H-isoquinoline chloride;
(4R)-4-methyl-2-(1-methylpyrazolo[3,4-b]pyridin-4-yl)-6-(2-piperazin-1-ylethoxy)-3,4-dihydro-1H-isoquinoline;
(4R)-4-methyl-2-(1-methylpyrazolo[3,4-b]pyridin-4-yl)-6-[1-(4-piperidyl)azetidin-3-yl]oxy-3,4-dihydro-1H-isoquinoline;
(4R)-4-methyl-2-(1-methylpyrazolo[3,4-b]pyridin-4-yl)-6-[[(7R,8aS)-1,2,3,4,6,7,8,8a-octahydropyrrolo[1,2-a]pyrazin-7-yl]oxy]-3,4-dihydro-1H-isoquinoline;
(4R)-4-methyl-2-(1-methylpyrazolo[3,4-b]pyridin-4-yl)-6-[[(2R)-1-methylpiperazin-2-yl]methoxy]-3,4-dihydro-1H-isoquinoline;
(4R)-4-methyl-2-(1-methylpyrazolo[3,4-b]pyridin-4-yl)-6-[[(2S)-1-methylpiperazin-2-yl]methoxy]-3,4-dihydro-1H-isoquinoline;
(2S)-2-[[(4R)-4-methyl-2-(1-methylpyrazolo[3,4-b]pyridin-4-yl)-3,4-dihydro-1H-isoquinolin-6-yl]oxymethyl]piperazine-1-carbaldehyde;
trans-4-amino-1-[(4R)-4-methyl-2-(1-methylpyrazolo[3,4-b]pyridin-4-yl)-3,4-dihydro-1H-isoquinolin-6-yl]pyrrolidin-3-ol;
trans-4-amino-1-[(4R)-2-(1,6-dimethylpyrazolo[3,4-b]pyridin-4-yl)-4-methyl-3,4-dihydro-1H-isoquinolin-6-yl]pyrrolidin-3-ol;
2-[(4R)-2-(1,6-dimethylpyrazolo[3,4-b]pyridin-4-yl)-4-methyl-3,4-dihydro-1H-isoquinolin-6-yl]-5-oxa-2,8-diazaspiro[3.5]nonane;
[4-[(4R)-4-methyl-2-(1-methylpyrazolo[3,4-b]pyridin-4-yl)-3,4-dihydro-1H-isoquinolin-6-yl]piperazin-2-yl]methanol;
[(2R)-4-[(4R)-4-methyl-2-(1-methylpyrazolo[3,4-b]pyridin-4-yl)-3,4-dihydro-1H-isoquinolin-6-yl]piperazin-2-yl]methanol;
[(2S)-4-[(4R)-4-methyl-2-(1-methylpyrazolo[3,4-b]pyridin-4-yl)-3,4-dihydro-1H-isoquinolin-6-yl]piperazin-2-yl]methanol;
2-[(4R)-4-methyl-2-(1-methylpyrazolo[3,4-b]pyridin-4-yl)-3,4-dihydro-1H-isoquinolin-6-yl]-2,7-diazaspiro[4.4]nonan-3-one;
(5S)-2-[(4R)-4-methyl-2-(1-methylpyrazolo[3,4-b]pyridin-4-yl)-3,4-dihydro-1H-isoquinolin-6-yl]-2,7-diazaspiro[4.4]nonan-3-one;
(5R)-2-[(4R)-4-methyl-2-(1-methylpyrazolo[3,4-b]pyridin-4-yl)-3,4-dihydro-1H-isoquinolin-6-yl]-2,7-diazaspiro[4.4]nonan-3-one;
cis-2-[(4R)-4-methyl-2-(1-methylpyrazolo[3,4-b]pyridin-4-yl)-3,4-dihydro-1H-isoquinolin-6-yl]-3a,4,5,6,7,7a-hexahydro-1H-pyrrolo[3,4-c]pyridin-3-one;
(3aS,7aS)-2-[(4R)-4-methyl-2-(1-methylpyrazolo[3,4-b]pyridin-4-yl)-3,4-dihydro-1H-isoquinolin-6-yl]-3a,4,5,6,7,7a-hexahydro-1H-pyrrolo[3,4-c]pyridin-3-one;
(3aR,7aR)-2-[(4R)-4-methyl-2-(1-methylpyrazolo[3,4-b]pyridin-4-yl)-3,4-dihydro-1H-isoquinolin-6-yl]-3a,4,5,6,7,7a-hexahydro-1H-pyrrolo[3,4-c]pyridin-3-one;
1-[(8S)-6-(1,6-dimethylpyrazolo[3,4-b]pyridin-4-yl)-8-methyl-7,8-dihydro-5H-1,6-naphthyridin-2-yl]piperidin-4-amine;
4-methyl-1-[(8S)-6-(1,6-dimethylpyrazolo[3,4-b]pyridin-4-yl)-8-methyl-7,8-dihydro-5H-1,6-naphthyridin-2-yl]piperidin-4-amine;
N,N-dimethyl-1-[(8S)-6-(1,6-dimethylpyrazolo[3,4-b]pyridin-4-yl)-8-methyl-7,8-dihydro-5H-1,6-naphthyridin-2-yl]piperidin-4-amine;
4-[(dimethylamino)methyl]-1-[(8S)-6-(1,6-dimethylpyrazolo[3,4-b]pyridin-4-yl)-8-methyl-7,8-dihydro-5H-1,6-naphthyridin-2-yl]piperidin-4-ol;
(8S)-8-methyl-6-(1-methylpyrazolo[3,4-b]pyridin-4-yl)-2-piperazin-1-yl-7,8-dihydro-5H-1,6-naphthyridine;
(3R,4R)-4-methoxy-1-[(8S)-6-(1,6-dimethylpyrazolo[3,4-b]pyridin-4-yl)-8-methyl-7,8-dihydro-5H-1,6-naphthyridin-2-yl]pyrrolidin-3-amine;
(8S)-6-(1,6-dimethylpyrazolo[3,4-b]pyridin-4-yl)-8-methyl-2-(4-piperidyloxy)-7,8-dihydro-5H-1,6-naphthyridine;
(3S,4S)-3-methoxy-1-[(8S)-6-(1,6-dimethylpyrazolo[3,4-b]pyridin-4-yl)-8-methyl-7,8-dihydro-5H-1,6-naphthyridin-2-yl]piperidin-4-amine;
(8S)-6-(1,6-dimethylpyrazolo[3,4-b]pyridin-4-yl)-8-methyl-2-piperazin-1-yl-7,8-dihydro-5H-1,6-naphthyridine;
(8S)-6-(1,6-dimethylpyrazolo[3,4-b]pyridin-4-yl)-8-methyl-2-(4-methylpiperazin-1-yl)-7,8-dihydro-5H-1,6-naphthyridine;
2-[(8S)-6-(1,6-dimethylpyrazolo[3,4-b]pyridin-4-yl)-8-methyl-7,8-dihydro-5H-1,6-naphthyridin-2-yl]-5-oxa-2,8-diazaspiro[3.5]nonane;
(3S,4R)-4-fluoro-1-[(8S)-6-(1,6-dimethylpyrazolo[3,4-b]pyridin-4-yl)-8-methyl-7,8-dihydro-5H-1,6-naphthyridin-2-yl]pyrrolidin-3-amine;
8-[(8S)-6-(1,6-dimethylpyrazolo[3,4-b]pyridin-4-yl)-8-methyl-7,8-dihydro-5H-1,6-naphthyridin-2-yl]-5-oxa-2,8-diazaspiro[3.5]nonane;
(8R)-6-(1,6-dimethylpyrazolo[3,4-b]pyridin-4-yl)-8-methyl-2-piperazin-1-yl-7,8-dihydro-5H-1,6-naphthyridine;
(3S,4S)-3-methoxy-1-[(5R)-7-(1,6-dimethylpyrazolo[3,4-b]pyridin-4-yl)-5-methyl-6,8-dihydro-5H-2,7-naphthyridin-3-yl]piperidin-4-amine;
(4R)-2-(1,6-dimethylpyrazolo[3,4-b]pyridin-4-yl)-4-methyl-6-piperazin-1-yl-3,4-dihydro-1H-2,7-naphthyridine;
(4R)-6-(4,7-diazaspiro[2.5]octan-7-yl)-2-(1,6-dimethylpyrazolo[3,4-b]pyridin-4-yl)-4-methyl-3,4-dihydro-1H-2,7-naphthyridine;
(4R)-2-(1,6-dimethylpyrazolo[3,4-b]pyridin-4-yl)-4-methyl-6-[[(7R,8aS)-1,2,3,4,6,7,8,8a-octahydropyrrolo[1,2-a]pyrazin-7-yl]oxy]-3,4-dihydro-1H-2,7-naphthyridine;
(4R)-2-(1,6-dimethylpyrazolo[3,4-b]pyridin-4-yl)-4-methyl-6-(2-piperazin-1-ylethoxy)-3,4-dihydro-1H-2,7-naphthyridine;
(3R,4R)-4-methoxy-1-[(5R)-7-(1,6-dimethylpyrazolo[3,4-b]pyridin-4-yl)-5-methyl-6,8-dihydro-5H-2,7-naphthyridin-3-yl]pyrrolidin-3-amine;
2-[(5R)-7-(1,6-dimethylpyrazolo[3,4-b]pyridin-4-yl)-5-methyl-6,8-dihydro-5H-2,7-naphthyridin-3-yl]-5-oxa-2,8-diazaspiro[3.5]nonane;
(3R,4R)-3-fluoro-1-[(5R)-7-(1,6-dimethylpyrazolo[3,4-b]pyridin-4-yl)-5-methyl-6,8-dihydro-5H-2,7-naphthyridin-3-yl]piperidin-4-amine; and
(4R)-2-(1,6-dimethylpyrazolo[3,4-b]pyridin-4-yl)-4-methyl-6-[(3R)-3-(methoxymethyl)piperazin-1-yl]-3,4-dihydro-1H-2,7-naphthyridine;
or a pharmaceutically acceptable salt thereof.

18. A process for the preparation of a compound according to claim 1 comprising the following:
a) a Buchwald-Hartwig C—N or C—O bond formation of a compound of formula (IV), 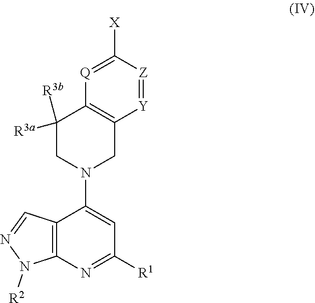
with R⁵·H, in the presence of a catalyst and a base;
wherein:
R⁵ is R⁴ or R⁴ with a protecting group, wherein the protecting group is selected from Boc, Cbz, acyl, sulfonyl, alkyl or aryl;
the catalyst is selected from RuPhos Pd G2, [Pd(allyl)Cl]₂/JackiePhos and Pd[P(o-tol)₃]₂/CyPF-t-Bu; and
the base is selected from Cs₂CO₃ and t-BuONa.

19. (canceled)

20. A pharmaceutical composition comprising a compound in accordance with claim 1 and a therapeutically inert carrier.

21. (canceled)

22. (canceled)

23. (canceled)

24. (canceled)

25. A compound or pharmaceutically acceptable salt, when manufactured according to the process of claim 18.

26. A method for the treatment or prophylaxis of systemic lupus erythematosus or lupus nephritis, which method comprises administering to a patient in need thereof a therapeutically effective amount of a compound as defined in claim 1.

27. (canceled)

28. A method for the treatment or prophylaxis of systemic lupus erythematosus or lupus nephritis, which method comprises administering to a patient in need thereof a therapeutically effective amount of a compound as defined in claim 17.

29. A pharmaceutical composition comprising a compound in accordance with claim 17 and a therapeutically inert carrier.
